(19) United States
(12) Patent Application Publication
(10) Pub. No.: US 2024/0288421 A1
(43) Pub. Date: Aug. 29, 2024

(54) TARGET MEASUREMENT

(71) Applicant: Kypha, Inc., St. Louis, MO (US)

(72) Inventors: Niamh O'Luanaigh, Dublin (IE); Martin Jefferson Schmidt, Raleigh, NC (US); Ronan Shaughnessy, Dublin (IE); Daniel Mills, Dublin (IE); Oksana Shkilnyk, Dublin (IE); Michelle Lynn Elvington, St. Louis, MO (US)

(21) Appl. No.: 18/289,794

(22) PCT Filed: May 5, 2022
(86) PCT No.: PCT/US22/27941

§ 371 Date: Nov. 7, 2023

Related U.S. Application Data
(60) Provisional application No. 63/185,696, filed on May 7, 2021.

(51) Int. Cl. G01N 33/543

(52) U.S. Cl. CPC G01N 33/54388; G01N 2333/4716; G01N 2333/5412; G01N 2333/5421; G01N 2333/96486

(57) ABSTRACT

The present disclosure provides, among other things, improved technologies for measuring one or more targets in a sample.

### RELATED APPLICATIONS

This application claims priority to U.S. Provisional Application No. 63/185,696, filed May 7, 2021, the contents of which are incorporated by reference herein in their entirety.

### BACKGROUND

Assaying one or more targets in one or more liquid samples can be prone to inaccuracies. Some contributing factors may be concentration of a target, type or condition of sample, and/or sample processing steps that need to be performed before measuring a target. There is a need for technologies that provide efficient, sensitive, specific, accurate, reliable, and/or rapid measurements for one or more targets in one or more samples.

### SUMMARY

The present disclosure provides technologies for measuring one or more targets from one or more liquid samples (e.g., undiluted samples, e.g., minimally-diluted samples). As will be appreciated by those of skill in the art, there are several sources of problems associated with measuring one or more targets in one or more liquid samples from a patient.

For example, the present disclosure provides a solution to a source of a problem in evaluating therapeutic drug/target interactions. In particular, the present disclosure provides insight that such problems may stem from post-collection, pre-measurement sample-handling procedures such as dilution, which is required in many known assays that are used to measure one or more targets. The present disclosure also recognizes that measuring certain targets and/or therapeutics in a sample can be prone to inaccuracies due to dissociation of a target from a therapeutic. For instance, when a sample is diluted in order for a target and/or therapeutic to be measured, dilution can result in dissociation of the target and therapeutic, resulting in falsely elevated measurements relative to prior to dilution.

Among other things, technologies provided herein stabilize target: therapeutic interactions and, also, provide means to efficiently, accurately, sensitively, specifically, and reliably, and/or rapidly measure a target. For example, in some embodiments, the present disclosure provides technologies that can measure a target in a sample using an undiluted or minimally diluted sample, which contacts a test strip within 6-10 seconds of application and migrates through the entire length of the test strip within 90 seconds. Such technologies provide improvements over previously available assays including by rapidly capturing target: therapeutic complexes before substantial dissociation and then accurately measuring a level of a target in a sample.

Furthermore, the present disclosure recognizes that inability to dilute a sample introduces difficulties in detection if a target is highly concentrated. That is, until now, high concentration targets have only been able to be measured by diluting a sample because oversaturation limited capabilities of previously available assays to accurately measure such targets. Also encompassed is the recognition that measuring multiple targets from a single sample (e.g., without dilution of the sample) was challenging prior to technologies of the present disclosure. That is, previously available assays were complicated by factors such as different targets traditionally requiring different assay conditions. For instance, if one target was a high concentration target and another target a low concentration target, previously available assays were not able to efficiently, accurately, specifically, sensitively, reliably, and/or rapidly measure both targets. In addition, measurements of certain types of targets (e.g., a biomarker, e.g., a complement protein, e.g., a complement-pathway protein, etc.) may be impossible, incomplete, and/or inaccurate due to inability to properly detect lower and/or upper limits of quantity in a given sample. In particular, the present disclosure recognizes and provides solutions to limitations of previously available assays. For instance, the present disclosure improves dynamic range of detection of a variety of targets, whereas previously available assays were unable to properly detect lower and upper limits of certain targets, capture a complete range of quantities for a given target, and/or accurately measure a target because of a disruptive processing step required to perform such assays (e.g., several serial dilutions). In some embodiments, provided technologies allow for accurate measurement of two or more targets substantially simultaneously. In some such embodiments, such measurements may require one or more of different processing or pre-processing steps or conditions, assay (e.g., buffer) conditions, handling conditions, and/or dilution(s) in a single assay, which is something not achievable using previously available assays. Among other things, the present disclosure provides technologies that provide solutions to these and other problems.

As mentioned above, the present disclosure provides the insight that many assays are inaccurately measuring one or more targets. For example, previously available assays may be erroneously failing to detect presence of a target, or under or over-reporting concentration of a target. Thus, the present disclosure reveals that prior art assays lack appropriate efficiency, specificity, accuracy, sensitivity, reliability and/or speed for measuring one or more targets. That is, prior art assays cannot accurately quantify a target across an appropriately wide-range of possible concentrations. For example, in some embodiments, certain targets may be present at such low or high levels that measurements are inaccurate because a target is erroneously not detected or concentration of a given target is over- or under-reported due to artifacts or limitations inherent to previously available assays such as combinations of dilution and target capture/detection for target measurement. In some embodiments, the present disclosure provides technologies that allow, for the first time, accurate measurement of one or more targets with upper or lower limits of quantitation that presently fall outside of ranges of detection of current assays, including embodiments directed to multiplexed assays. In some embodiments, a target is so abundant that currently available methods cannot capture or report within appropriate upper or lower limits of detection to quantify a given target (e.g., an abundant target) in a sample. In some embodiments, a target is present in such a low concentration that appropriate detection (e.g., with accuracy, sensitivity, and specificity) and/or quantification has not been possible prior to the technologies provided by the present disclosure.

In addition to achieving unprecedented combinations of efficiency, sensitivity, specificity, accuracy, reliability and/or speed, technologies of the present disclosure also provide a platform that can be used in a point-of-care application, reducing handling, number of personnel required and amount of time to assay results. Importantly, such features provide platforms upon which clinicians and providers can improve patient care by rapidly and easily detecting and monitoring a patient for one or more targets and preventing and/or treating one or more conditions that, prior to the technologies of the present disclosure, may have had disastrous outcomes due to inaccuracy or inability to detect underlying and ongoing changes in one or more targets. The present disclosure provides insights and new technologies including assay methods that will, for the first time, allow efficiently, specific, sensitive, accurate, reliable, and/or rapid measurement of one or more targets. Such technologies will allow safer and more effective development and monitoring of therapeutics as well as safer and more effective treatment of patients.

In some aspects, the present disclosure provides a method of measuring a target comprising: (i) obtaining a sample; (ii) contacting an immunoassay device with at least a portion of the sample; and (iii) and measuring one or more targets in the sample.

In some aspects the present disclosure provides a method of measuring at least one target in a sample comprising an improvement (e.g., relative to previously available assays), which improvement comprises measuring the target in a sample by contacting an immunoassay device with a sample that has not been subjected to an offline dilution prior to the contacting, and measuring one or more targets, wherein the measurements are more efficient, accurate, sensitive, specific, reliable, and/or rapid as compared to measuring performed with a diluted sample. In some such embodiments, at least one measurement is more efficient, accurate, sensitive, specific, reliable, and/or rapid than a method that comprises one or more offline sample dilution steps prior to the contacting the immunoassay device.

In some embodiments, an immunoassay device comprises at least one test strip.

In some embodiments, an immunoassay device is contacted with a portion of the sample within 30 minutes of a sample being obtained.

In some embodiments, a sample is not subjected to an offline dilution prior to contacting an immunoassay device.

In some embodiments, prior to contacting an immunoassay device, a sample is subjected to a pre-processing step comprising at least one dilution.

In some embodiments, a sample is or comprises fluid. In some embodiments, a sample is not solid or substantially comprised of solid material. In some embodiments, a sample is or comprises whole blood, plasma, serum, aqueous humor, tears, ocular fluid, urine and/or cerebrospinal fluid. In some embodiments, a sample is a crude sample. In some such embodiments, a crude sample has not been diluted prior to contacting the immunoassay device.

In some embodiments, a sample has undergone one or more purification steps prior to contacting the immunoassay device.

In some embodiments, an immunoassay device of the present disclosure comprises at least one of a sample pad and conjugate pad.

In some embodiments, an immunoassay device comprises at least two, three, four, five, six, seven, eight or more test strips. In some such embodiments, at least two, three, or four test strips each comprises at least two, three, four, five, six or more test lines.

In some embodiments, a test line comprises at least one capture agent. In some such embodiments, a capture agent is or comprises an antibody.

In some embodiments, measurements of one or more targets of the present disclosure is/are performed between approximately 30-300 minutes after the sample is obtained. In some embodiments, measuring is performed no greater than 30 minutes after the sample is obtained.

In some aspects, the present disclosure provides a method comprising: (a) measuring a target in a sample comprising steps of: (i) obtaining the sample; (ii) contacting an immunoassay device with at least a portion of the sample; (iii) allowing the sample and the immunoassay device to sit together for a period of time; (iv) measuring at least one target in the sample; and (b) comparing the measurement of one or more targets to at least one reference measurement; and (c) optionally changing or administering one or more treatments to a subject from whom the sample was obtained.

In some embodiments, an immunoassay device comprises at least one test strip. In some such embodiments, a test strip comprises at least one test line comprising beads. In some embodiments, an immunoassay device comprises at least one conjugate pad. In some embodiments, a test strip comprises at least one conjugate pad comprising beads. In some embodiments, beads comprise nanobeads (e.g. polysytrene beads, colloidal beads, etc). In some embodiments, nanobeads have a diameter between 100-500 nm. In some embodiments, nanobeads have a diameter between 200-400 nm.

In some embodiments, nanobeads comprise at least one detecting agent. In some embodiments, a detecting agent is an antibody (e.g., a labeled antibody). In some embodiments, a detecting agent is a protein or peptide (for e.g. competition immunoassays). In some embodiments, a detecting agent is or comprises a low molecular weight fluorophore.

In some embodiments, an immunoassay device comprises at least one competing agent and/or at least one capture agent.

In some embodiments, a competing agent is or comprises an antibody.

In some embodiments, a capture agent is or comprises an antibody.

In some embodiments, an immunoassay device comprises at least one of a conjugate pad and sample pad.

In some embodiments, a conjugate pad comprises at least one competing agent and/or at least one detecting agent. In some such embodiments, a competing agent binds to at least one target in the sample. In some such embodiments, a detecting agent binds to at least one target in the sample.

In some embodiments, a competing agent binds to an excess amount of at least one target in a sample.

In some embodiments, measuring of a target is performed using a reader system capable of measuring multiple test lines in multiple visualizable channels.

In some aspects, the present disclosure provides a method comprising: (a) measuring an amount of a target in a sample with steps comprising: (i) obtaining a sample, wherein the sample is diluted in a vessel; (ii) allowing the vessel with the diluted sample to sit for a first period of time; (iii) contacting an immunoassay device with the diluted sample; (iv) allowing the immunoassay device comprising the diluted sample to sit for a second period of time; (v) measuring at least one target in the sample; and (b) optionally changing or administering one or more treatments to a subject from whom the sample was obtained.

In some embodiments, a first period of time is no longer than five minutes. In some embodiments, a second period of time is no longer than five minutes.

In some embodiments, a sample is diluted with a substrate with which at least one target in the sample can react.

In some embodiments, the present disclosure provides a kit comprising: (i) a test cassette comprising an immunoassay device, which immunoassay device comprises at least one test strip comprising at least one test line; (ii) at least one detecting agent; and, optionally (iii) at least one capture agent.

In some embodiments, the present disclosure provides a test cassette comprising: (i) an immunoassay device, which itself comprises at least one test strip and (ii) at least two test lines.

In some embodiments, the present disclosure provides a method of diagnosing a subject as having or being susceptible to having at least one disease, disorder, or condition comprising: (i) obtaining a sample from the subject; (ii) measuring one or more targets in the sample; (iii) comparing the measurement of one or more targets to a measurement range of one or more reference samples; and (iv) diagnosing a patient as having or being at risk of developing a disease, disorder or condition if a measurement of the one or more targets is outside of the measurement range of the one or more reference samples.

In some embodiments, the present disclosure provides a reader system comprising: (a) means for measuring one or more targets in a sample comprising (i) inserting a test cassette into the reader system such that the direction of sample flow on an immunoassay device of the test cassette is oriented parallel with gravity (ii) measuring at least one target on at least one test line on at least one test strip in the immunoassay device. In some such embodiments, measuring comprises measuring at least four test lines on at least one test strip of an immunoassay device.

In some embodiments, a test cassette is inserted via a port in a reader system. In some such embodiments, a sample is added to the immunoassay device prior to insertion of the test cassette into the reader system. In some embodiments, a sample is added to the immunoassay device after insertion of the test cassette into the reader system. In some embodiments, the measuring comprises measuring at least four test lines on at least one test strip of the immunoassay device.

In some embodiments, a target of the present disclosure is one or more of C3, C3a, iC3b, C4, C5, sC5b-9, IL-6, ADAMTS13, MASP2:AT complex, C4d, Ba, Bb, FH, CXCL9, sCD25, microRNA, IL8, Pentraxin3, IL1, VCAM1, thrombomodulin, ferritin, CRP, IL-10, TNFα, IFNγ, and/or creatinine. In some embodiments, a test line comprises at least one capture agent for one or more of C3, C3a, iC3b, C4, C5, sC5b-9, IL-6, ADAMTS13, MASP2:AT complex, C4d, Ba, Bb, FH, CXCL9, sCD25, microRNA, IL8, Pentraxin3, IL1, VCAM1, thrombomodulin, ferritin, CRP, IL-10, TNFα, IFNγ, and/or creatinine.

In some embodiments of the present disclosure a subject is suspected or at risk of having one or more of age-related macular degeneration (AMD), complement 3 glomerulopathy (C3G), Hematopoietic Stem Cell Transplant-Associated Thrombotic Microangiopathy (HSCT-TMA), Complement-Mediated Thrombotic Microangiopathy (CM-TMA), atypical hemolytic uremic syndrome (aHUS), thrombotic thrombocytopeniarpura (TTP), COVID19, lupus erythematosus, lupus nephritis, cytokine release syndrome, Alzheimer's Disease (AD), or combinations thereof.

In some embodiments, a subject of the present disclosure is diagnosed as at risk of developing or as having one or more of age-related macular degeneration (AMD), complement 3 glomerulopathy (C3G), Hematopoietic Stem Cell Transplant-Associated Thrombotic Microangiopathy (HSCT-TMA), Complement-Mediated Thrombotic Microangiopathy (CM-TMA), atypical hemolytic uremic syndrome (aHUS), thrombotic thrombocytopenic purpura (TTP), COVID19, lupus erythematosus, lupus nephritis, cytokine release syndrome, Alzheimer's Disease (AD), or combinations thereof.

### BRIEF DESCRIPTION OF THE DRAWING

FIG. 1 provides a schematic overview of the complement system.

FIG. 2A shows an exemplary test cassette setup and certain components of an exemplary embodiment as disclosed herein.

FIG. 2B shows an exemplary experiment used in developing an exemplary immunoassay device comprising multiple test strips with multiple test lines and that provides improved measurements of one or more targets.

FIG. 3 shows top and side views of components of an exemplary test cassette configuration of the present disclosure.

FIG. 4 shows an exemplary test strip configuration in an immunoassay device for use in a test cassette of present disclosure.

FIG. 5 shows an exemplary test strip configuration in an immunoassay device for use in a test cassette of the present disclosure.

FIGS. 6A and 6B show two different assays for quantitation of a target each using beads of different materials and sizes.

FIG. 7A demonstrates results used to identify a source of a problem in measuring an exemplary high abundance target in a sample and a high abundance target in presence of a therapeutic. FIG. 7B demonstrates a solution developed and disclosed herein to overcome inaccuracies in measurements in high abundance targets and FIG. 7C shows accurate measuring of free versus therapeutic bound-target using methods as developed herein.

FIG. 8 shows results using a multiline approach for quantitation of a high concentration exemplary target in a sample.

FIG. 9 shows results from an assay using a prozone reduction method.

FIG. 10 shows results from an assay using a prozone reduction method.

FIG. 11 shows a comparison of cassettes results from an assay using with test strips comprising different pore sizes to eliminate a filtering effect.

FIG. 12 shows results from an assay measuring a high concentration exemplary target.

FIG. 13 shows a comparison of results measuring an exemplary target using technologies of the present disclosure as compared to those from a standard ELISA.

FIG. 14 shows test line peaks on two different test lines at various concentrations of an exemplary target.

FIG. 15 shows results of an exemplary low concentration target from banked samples of patients with an autoimmune disease.

FIG. 16A and FIG. 16B shows results measuring the capture and detection specificity of an exemplary target at various concentrations using technologies of the present disclosure.

FIG. 17 shows exemplary results comparing classical complement pathway activation from stimulated and unstimulated plasma samples at various time points using technologies of the present disclosure.

FIG. 18 shows sC5b-9 levels in urine from patients between visits in which a high renal score versus a low renal score was recorded.

### DETAILED DESCRIPTION

### Definitions

Administration: as used herein, “administration” refers to the administration of a treatment to a subject or a system. In some embodiments, administration may be of a composition to a subject or system. In some embodiments, administration to an animal subject (e.g., to a human) may be by any appropriate route. For example, in some embodiments, administration (e.g., of a composition) may be bronchial (including by bronchial instillation), buccal, enteral, interdermal, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (including by intratracheal instillation), transdermal, vaginal and vitreal. In some embodiments, administration may involve intermittent dosing. In some embodiments, administration may involve continuous dosing (e.g., perfusion) for at least a selected period of time.

Agent: as used herein, “agent” may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present disclosure include small molecules, antibodies, antibody fragments, complement receptors or binding proteins, enzymes, aptamers, nucleic acids (e.g., siRNAs, shRNAs, DNA/RNA hybrids, antisense oligonucleotides, ribozymes), peptides, peptide mimetics, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety. In some embodiments, an agent is used to detect and/or report (e.g., through visualizable techniques) presence or absence of one or more targets in a sample (e.g., a detecting agent). In some embodiments, an agent is used to compete with e.g., another agent and/or bind with one or more targets in a sample (e.g., a competing agent, e.g., a capture agent). In some embodiments, a difference between a competing agent and a capture agent is location on a test cassette. For example, in some embodiments, a competing agent is initially localized to a conjugate pad and upon sample contact, the competing agent binds to a target in the sample. In some such embodiments, the competing agent: target complex is not necessarily retained in the conjugate pad but can pass through a membrane of an immunoassay device. In some embodiments, a capture agent is localized to a test line and upon sample contact with a test line, a target is immobilized to the test line with the capture agent. In some such embodiments, the “captured” target associates (e.g., forms a complex with) a detection agent, which can be visualized on a test line in an immunoassay device. In some embodiments, a difference between a detecting agent and a competing agent is presence of a moiety detectable by a reader system or otherwise observable by examining the test strip. That is, in some embodiments, a competing agent may become a detecting agent by addition of a detectable moiety. In some embodiments, a detecting agent is also a competing agent. In some embodiments, a competing agent is not a detecting agent. In some embodiments, a competing agent and a capture agent may be the same agent, but a competing agent is localized to a conjugate pad and a capture agent is localized to a test line of an immunoassay device of the present disclosure. As described herein, in some embodiments, competing, capture, and/or detecting agents are target-specific.

Approximately: as used herein, “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: as used herein, two events or entities are “associated” with one another, if the presence, level and/or form of one is correlated with that of the other. For example, a particular entity (e.g., polypeptide, genetic signature, metabolite, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biomarker: as used herein, “biomarker,” consistent with its use in the art, refers to an entity whose presence (or absence), level, or form, correlates or is associated with a particular biological event or state of interest, so that it is considered to be a “marker” of that event or state. To give but a few examples, in some embodiments, a biomarker may be or comprise a marker for a particular disease state, or for likelihood that a particular disease, disorder, or condition may develop (e.g., cytokine release syndrome, lupus erythematosus, aHUS, etc.). In some embodiments, a biomarker may be or comprise a marker for a particular disease or therapeutic outcome, or likelihood thereof. Thus, in some embodiments, a biomarker is predictive, in some embodiments, a biomarker is prognostic, in some embodiments, a biomarker is diagnostic, of the relevant biological event or state of interest. A biomarker may be an entity of any chemical class. For example, in some embodiments, a biomarker may be or comprise a nucleic acid, a polypeptide, a lipid, a carbohydrate, a small molecule, an inorganic agent (e.g., a metal or ion), a metabolite, or a combination thereof. In some embodiments, a biomarker is a cell surface marker. In some embodiments, a biomarker is intracellular. In some embodiments, a biomarker is found outside of cells (e.g., is secreted or is otherwise generated or present outside of cells, e.g., in a body fluid such as blood, urine, tears, saliva, cerebrospinal fluid, breath condensate, etc. In some embodiments, a biomarker is measured using a fluid sample from a subject. For example, in some embodiments, a biomarker is measured in a sample comprising one or more of blood, plasma, urine, tears, saliva, cerebrospinal fluid, etc. In some embodiments, one or more biomarkers is measured, and results are compiled into a panel and interpreted to determine, e.g., likelihood that a disease, disorder, or condition exists or is likely to exist or develop. In some embodiments, a target may be or comprise a biomarker. In some embodiments, a biomarker may be or comprise a target.

Combination therapy: as used herein, “combination therapy” refers to those situations in which a subject is simultaneously exposed to two or more therapeutic regimens (e.g., two or more therapeutic agents). In some embodiments, two or more agents or may be administered simultaneously; in some embodiments, such agents may be administered sequentially; in some embodiments, such agents are administered in overlapping dosing regimens.

Comparable: as used herein, “comparable” refers to two or more agents, entities, situations, sets of conditions, etc., that may not be identical to one another but that are sufficiently similar to permit comparison there between so that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Control line: as used herein, “control line” is a test line that is deposited (i.e., striped) on a test strip that serves as an internal quality control. That is, the control line indicates that the strip and/or reader system (e.g., an immunoassay reader system) are both functioning within acceptable parameters. In some embodiments, a test strip has a control line and one or more additional test lines. In some embodiments, a test strip has only a control line (e.g., a control test cassette, e.g., only one test line). In some embodiments, a control line comprises one or more components which can capture or bind to, in some configuration, one or more controls in a sample (e.g., a subject sample, e.g., a control sample provided in a kit, etc.).

Detecting: as used herein, “detecting” refers to ability to identify absence, presence, and/or level of a particular entity such as an agent or target, as described herein. For example, in some embodiments, detecting identifies presence of a target in a sample through, e.g., use of one or more signals, e.g., visualizable signals. In some embodiments, detecting may also be or comprise absence of one or more signals indicating absence of a particular agent or target in a sample. In some embodiments, detecting is qualitative (e.g., identifies presence or absence). In some embodiments, detecting is quantitative (e.g., identifies a particular amount or concentration of an entity). In some embodiments, a detecting agent is a specific for a given target.

Determine or Determining: as used herein, many provided methodologies described herein include a step of “determining”. Those of ordinary skill in the art, reading the present disclosure, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Dilute or dilution: As used herein, “dilute” or “dilution” refers to a process of decreasing the concentration of one or more components relative to the undiluted sample. In some embodiments, a dilution involves adding a liquid to achieve a decrease in concentration. In some embodiments, a dilution may be an inline dilution. In some embodiments, a dilution may be an offline dilution.

Dosing regimen: (or “therapeutic regimen”), as used herein, “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Flare: as used herein, “flare” indicates an acute increase in severity of symptoms of a disease, disorder, or condition sufficient to cause a clinician to initiate or alter treatment in a subject. In some embodiments, a flare may be defined as achieving a certain score on one or more disease indices, for example the SELENA SLEDAI Flare Index or the Physician Global Assessment (PGA). In some embodiments, an episodic change of disease activity indicates a flare. In some embodiments, a flare may be characterized or defined by new or increased use of treatments such as, for example, high dose corticosteroids (e.g., prednisone dosed at greater than 20 mg/day) or immunosuppressives. For example, in some embodiments, a flare may be characterized or defined by hospitalization or death due to a disease, disorder or condition. In some embodiments, a flare may be or comprise a measurable increase in disease activity in one or more organ systems involving new or worse clinical signs and symptoms and/or laboratory measurements, for example, as compared to a previously taken measurement. In some embodiments, a measurable increase in disease activity is considered clinically significant by the assessor and typically includes at least consideration of a change or an increase in treatment and, quite often, implementing a change in treatment. In some embodiments, flare is characterized by or defined as a change of greater than or equal to 1.0 in the physician's global assessment of disease activity (measured on a 0-3) scale from the previous visit or from a visit within the last 200 days (e.g., last 100 days, e.g., last 93 days, e.g., last 75 days, e.g., last 50 days, e.g., last 25 days, e.g., last 10 days, e.g., last 5 days, e.g., last 1 day). In some embodiments, a flare may be a rapid and acute event, for example, within minutes (e.g., 10, 20, 30 or more) or hours (e.g., 1, 2, 3, 4, 5, 6 or more) from an inciting event (e.g., administration of a gene therapy treatment, e.g., administration of a CAR-T treatment, etc.)

“Improve,” “increase” or “reduce”: as used herein, “improve,” “increase” or “reduce” or grammatical equivalents thereof, indicate values that are relative to a baseline measurement, such as a measurement in the same individual prior to initiation of a treatment described herein, or a measurement in a control individual (or multiple control individuals) in the absence of the treatment described herein. In some embodiments, a “control individual” is an individual afflicted with the same form of disease or injury as an individual being treated.

Immunoassay device: as used herein, an “immunoassay device” is or comprises one or more test strips upon which a sample is applied and can then be evaluated such as for purposes of measuring one or more targets in the sample. In some embodiments, an immunoassay device further comprises at least one additional component. In some such embodiments, the at least one additional component is or comprises a sample pad and/or a conjugate pad. In some embodiments, an immunoassay device is housed within a casing. In some such embodiments, an immunoassay device housed, at least partially, within a casing, may be referred to a test cassette or test cartridge.

Inline dilution: as used herein an “inline dilution” refers to effectively diluting a sample of the present disclosure by applying at least a portion of the sample to an immunoassay device as described herein such that at least one portion of the immunoassay device comprises a solid phase that alters the sample in some way (e.g., removes certain cell types, captures a portion of a particular target, etc.), effectively diluting, in some way, at least some portion (e.g., a target or cell type) from a given sample.

In vitro: as used herein “in vitro” refers to events that occur in an artificial environment, e.g., in a test tube or reaction vessel, in cell culture, etc., rather than within a multi-cellular organism.

In vivo: as used herein, “in vivo” refers to events that occur within a multi-cellular organism, such as a human and a non-human animal. In the context of cell-based systems, the term may be used to refer to events that occur within a living cell (as opposed to, for example, in vitro systems).

Measure: as used herein, “measure” refers to a process of determining amount (e.g., quantitative) and/or degree of presence or absence (e.g., qualitative) of a target. In some embodiments, a measurement is made using technologies of the present disclosure. In some embodiments, technologies of the present disclosure are assayed using known instruments or devices to take one or more measurements. In some embodiments, a measurement is quantitative. In some embodiments, a measurement is qualitative. In some embodiments, a measurement includes a qualitative assessment of “absent.” In some embodiments, a measurement includes a quantitative assessment of “zero” or below a LLOQ.

Offline dilution: as used herein, “offline dilution” refers to a dilution performed with or to a sample prior to any portion of the sample being applied to an immunoassay device of the present disclosure. For example, in some embodiments, an offline dilution involves collecting a sample and diluting a sample by a particular set of steps and quantities. In some embodiments, a sample is diluted using a buffer. In some embodiments, a sample is diluted during performance of a step of an assay (e.g., an enzyme assay, e.g., incubation with a substrate, e.g., an ADAMTS13 activity assay, etc.). In some embodiments, a sample of the present disclosure is not subjected to any offline dilutions. In some embodiments, a sample of the present disclosure may be subject to at least one offline dilution. In some such embodiments, an offline dilution is a “minimal” dilution. In some embodiments, a minimal dilution does not comprise serial dilutions of a particular sample.

Prevention: as used herein, “prevention” refers to a delay of onset, and/or reduction in frequency and/or severity of one or more symptoms of a particular disease, disorder or condition. In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition. In some embodiments, prevention may be said to occur when onset of a disease, disorder or condition has been delayed for a predefined period of time. In some embodiments, prevention may be said to occur when onset of a disease, disorder or condition has been stopped from progressing, either fully, or at a particular rate relative to prior to prevention having occurred. For example, in some embodiments, technologies of the present disclosure may measure and reveal, in real-time, sensitive, accurate, reliable, and/or specific measurements of one or more targets and ability of a provider to see changes, in, for example, real-time and/or point-of-care allows one or more treatments to be administered. In some such embodiments, such measurements may fully prevent onset of a disease, disorder or condition (e.g., cytokine release syndrome that may occur with certain therapies such as, e.g., gene therapy, CAR-T therapy, etc.). In some embodiments, prevention may be said to occur when detection of one or more changes in one or more targets allows a provider to administer a treatment such that an outcome that would have occurred in absence of detection is mitigated, to some degree, or completely stopped as compared to if no treatment had occurred.

Pre-Process: as used herein, “pre-process” describes a process of treating a sample after obtaining or collecting the sample and prior to measuring one or more targets. In some embodiments, pre-process comprises contacting a sample with a liquid, which may optionally comprise at least one agent such that the sample and agent are mixed. In some such embodiments, such a mixing step results in dilution of the sample and, thus, of one or more targets.

Reference: as used herein, “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. In some such embodiments, the historical reference is a measurement taken from the same subject at a different point in time. In some embodiments, more than one reference value may be used; that is, for example, in some embodiments, a reference level from a standardized laboratory reference range as well as a reference level from a previous measurement of a given target conducted in the same subject may each be used to compare a measurement of a target in a subject. In some embodiments, a reference is in relation to a particular quantity of starting material as compared to an ending material after a process is performed. For example, in an enzymatic assay, a known quantity is input into an assay and a measurement may be of a percent of completion of a particular reaction versus starting material and hypothetical/theoretical amounts versus actual amounts; in some such embodiments, a reference is a hypothetical/theoretically calculated or modeled amount. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Response: as used herein, a “response” (e.g., to treatment) may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. The exact response criteria can be selected in any appropriate manner, provided that the groups to be compared are assessed based on the same or comparable criteria for determining response rate. One of ordinary skill in the art will be able to select appropriate criteria.

Risk: as will be understood from context, “risk” of a disease, disorder, and/or condition comprises likelihood that a particular individual will develop a disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more. In some embodiments, risk is present, but is not quantified by any particular number or index of risk, rather, risk is considered to exist when a measurement of one or more targets differs within or above a particular range for one or more particular targets relative to a reference range.

Sample: as used herein, “sample” typically refers to a biological sample obtained or derived from, in some manner (including, e.g., a commercially available source), a source of interest, as described herein. In some embodiments, a source of interest comprises an organism, such as an animal or human. In some embodiments, a sample comprises one or more targets to be measured by technologies of the present disclosure. In some embodiments, a sample does not comprise one or more targets to be measured by technologies of the present disclosure (e.g., a level of a target is zero or below a LLOQ). In some embodiments, a sample may be or comprise a control substance used for purposes of verifying, confirming, and/or calibrating one or more steps of measuring one or more targets using an immunoassay device as described herein.

Subject: as used herein, “subject” or “patient” is an animal. In some embodiments, a subject is a mammal. In some embodiments, the mammal is a rat, mouse, cat, dog, pig, non-human primate, etc. or prenatal forms thereof. In some embodiments, the mammal is a human. In some embodiments, a human includes prenatal human forms (e.g., an embryo, fetus). In some embodiments, a human is a neonate, infant, toddler, child, adolescent, or adult. In some embodiments, a human is an elderly adult. In some embodiments, a subject is suffering from a relevant disease, disorder or condition. In some embodiments, a subject is susceptible to (at risk of developing) a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is a control subject. In some embodiments, a subject is a participant in a trial, e.g., a research trial, e.g., a clinical trial. In some embodiments, a subject is an individual to whom diagnosis and/or therapy is and/or has been administered.

Substantially: as used herein, “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target: as used herein, a “target” is or comprises a particular entity that is being measured. A target may be any measurable entity that exists, at any level, endogenously or through exogenous introduction, in a sample. For example, in some embodiments, a target is a naturally occurring substance or agent (e.g., an endogenous protein). In some embodiments, a target is or comprises a biomarker. In some embodiments, a target is or comprises an analyte. In some embodiments, a target is or comprises a therapeutic. In some embodiments, a target is a high abundance target (e.g., relative to one or more other targets or to a control level of the target). In some embodiments, a target is a low abundance target (e.g., occurs at pg/mL concentrations). In some embodiments, a target is measured indirectly, such as through reaction with a substrate and quantification of substrate consumption, cleavage (e.g., of substrate) or other detectable modification.

Test Cassette or Test Cartridge: as used herein “test cassette” or “test cartridge” refers to a unit that comprises a housing, which housing comprises, at least partially, an immunoassay device as described herein. In some embodiments, a test cassette or cartridge comprises barcode or other means for reading or scanning identity of the cassette or cartridge by the device used to receive and interpret information from the immunoassay device. In some embodiments, a test cassette is a control test cassette. In some such embodiments, a control test cassette is provided to validate or confirm one or more aspects of one or more assays as described herein.

Test Line: as used herein, “test line” refers to a line deposited (i.e., striped) on a test strip. A test line comprises one or more components that can capture or bind to, in some configuration, one or more targets in a sample. For instance, in some embodiments, a test line comprises an agent. In some embodiments, a test line comprises a capture agent. In some embodiments, when a target is bound to a capture agent on a test line, a test line may also comprise a detecting agent. As will be appreciated by one of skill in the art, a capture agent and a competing agent may be the same agent, but at a test line, where a target is retained, such an agent is a capture agent. In some embodiments, a test line does not comprise a competing agent. In some embodiments, a test line does not comprise a detecting agent. In some embodiments, a test line is measured qualitatively and/or quantitatively. In some embodiments, and as will be understood from context, a test line functions as a control line as described herein. In some such embodiments, a capture agent on a test line may be different than a capture agent on a control line; for example, in some embodiments, a capture agent on a control line may be specific for a detector agent, whereas a capture agent on a test line is specific for a target (which target may be complexed with detector agent). In some embodiments, a test line is not measured (i.e., even if visualizable).

Test Strip: as used herein, “test strip” is a solid phase component of an immunoassay device described herein, which solid phase comprises at least one test line. In some embodiments, a test strip is contacted with a sample and the sample travels into and through the solid phase, flowing laterally using capillary and/or gravitational mechanisms.

Therapeutic agent: as used herein, “therapeutic agent” generally refers to any agent that elicits a desired effect when administered to an organism. By way of non-limiting example, in some embodiments, a desired effect may be one or more of a reduction in one or more targets, an increase in one or more targets, a decrease or resolution in one or more symptoms of one or more diseases, disorders, or conditions. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a therapeutic agent is an agent that has been or is required to be approved by a government agency before it can be marketed for administration to humans. In some embodiments, a therapeutic agent is an agent for which a medical prescription is required for administration to humans. In some embodiments, a therapeutic agent is one that is being tested and/or administered during research and development. In some embodiments, a therapeutic agent is one that is being tested and/or administered to a subject as part of a preclinical or clinical trial.

Therapeutic regimen: a “therapeutic regimen”, as that term is used herein, refers to a dosing regimen whose administration across a relevant population may be correlated with a desired or beneficial therapeutic outcome.

Treatment: as used herein, “treatment” (also “treat” or “treating”) refers to any administration of a procedure, intervention, substance (e.g., a corticosteroid) or any combination thereof that partially or completely alleviates, ameliorates, relives, inhibits, delays onset of, reduces severity of, and/or reduces incidence of one or more symptoms, features, and/or causes of a particular disease, disorder, and/or condition (e.g., cytokine release syndrome, lupus nephritis, aHUS, etc.). For example, in some embodiments, a treatment may comprise an intervention that includes withdrawal or removal of a substance (e.g., from a subject). In some embodiments, administration of a treatment may comprise removal of a composition and/or addition of a different composition and/or intervention. In some embodiments, treatment may be of a subject who does not exhibit overt signs of the relevant disease, disorder and/or condition. In some embodiments, treatment may be of a subject who exhibits only early signs of the disease, disorder, and/or condition. Alternatively or additionally, in some embodiments, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### DETAILED DESCRIPTION

### The Complement System

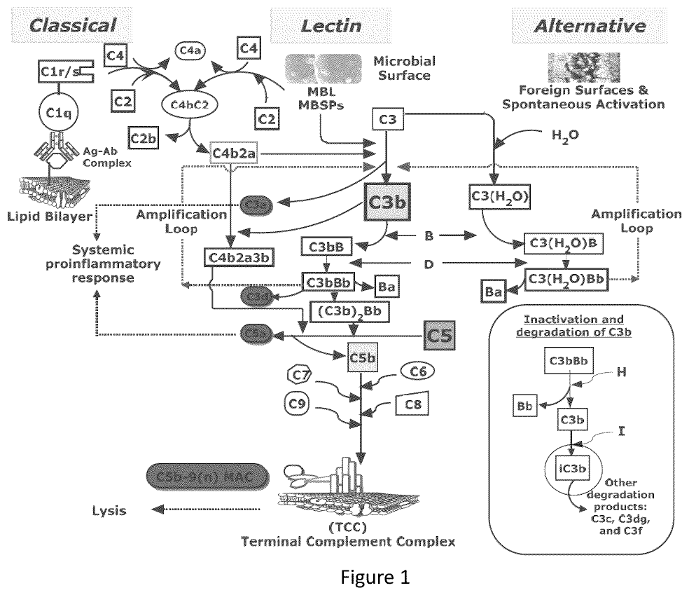

The complement system comprises more than 50 serum and cellular proteins and plays important roles in innate and adaptive immunity. As those skilled in the art are aware, there are three major pathways of complement activation: classical, lectin, and alternative. All three major pathways of complement activation converge on the central protein complement component 3 (C3). C3 is a central mediator of inflammation and is activated by most factors that cause inflammation. An exemplary schematic of a complement system and its pathways is shown in FIG. 1.

Complement and complement activation has been associated with a wide variety of diseases, and, in some cases, complement can play a role in disease pathology. In these cases, the body of an organism is not able to successfully control one or more causes of inflammation, which inflammation may then transform from local (or focal) to systemic. Complement activation may directly damage tissue or may cause damage indirectly by over-activating cells and recruiting immune cells that in turn cause tissue destruction. By way of non-limiting example, some conditions that may result from complement-mediated systemic over activation include anaphylactic shock, multiple organ failure (MOF), acute respiratory distress syndrome (ARDS), systemic inflammatory response syndrome (SIRS), and cytokine release syndrome (CRS).

C5 convertase generated via any of the three pathways cleaves C5 to produce C5a and C5b. C5b then binds to C6, C7, and C8, which catalyzes polymerization of C9 to form the C5b-9 membrane attack complex (MAC). The assembling MAC inserts itself into target cell membrane, forming a pore delineated by a ring of C9 molecules. MAC formation causes cell lysis of invading microbes, MAC formation on host cells can also cause lysis, but not necessarily. Sublytic amounts of MAC on the membrane of cells may affect cell function in a variety of ways. The small cleavage products C3a, C4a, and C5a are anaphylatoxins and mediate multiple reactions in the acute inflammatory response. C3a and C5a are also potent chemotactic factors that attract cells such as neutrophils and macrophages into the area of crisis.

### Classical

The classical complement pathway is understood to be primarily activated by immune complexes, including, specifically, IgG/IgM antibodies bound to antigen. Other activators can include, e.g., lipopolysaccharide (LPS), myelin, phospholipids, polyanionic compounds, C-reactive protein (CRP), Pentraxin3 (PTX3), serum amyloid component P (SAP), and microbial DNA and RNA, and any components or portions thereof (e.g., myelin proteins or fragments, LPS fragments, DNA fragments, etc.). The classical pathway is typically triggered by immune complexes, which are complexes of antigen bound with antibodies, generally belonging to the IgM or IgG isotypes. Immune complexes in turn bind to complement component C1, which is comprised of C1q, C1r, and C1s. The binding of C1q to an antibody-antigen complex triggers activation of C1r and C1s. Activated C1s then cleaves component C4 to produce C4a and C4b. C4b is capable of covalent attachment to cell surfaces, although only about five percent does so. The remaining 95 percent reacts with water to form a soluble, activated C4b. Component C2 can then associate with deposited C4b, after which it is activated by C1s to C2a and C2b. C4b and C2a combine to form C4bC2a, the classical pathway (CP) C3 convertase.

The CP convertase cleaves C3 to form C3a and C3b. Like activated C4b, C3b can covalently bind to cell surfaces or react with H₂O and stay in solution. Activated C3b has multiple roles. By itself, it can serve as an opsonin to make the decorated cell or particle more easily ingested by phagocytes. In addition, C3b can associate with C4bC2a (the CP C3 convertase) to form a C5 convertase. The complex, termed C4bC2aC3b is termed the CP C5 convertase. Alternatively, surface-bound C3b can form the core of another C3 convertase called the alternative pathway (AP) C3 convertase.

### Alternative

The alternative pathway is mediated by direct C3 activation by “foreign” substances that often include microbial cell wall components. The alternative pathway (AP) is another mechanism by which C3 can become activated. It is typically activated by targets such as microbial surfaces and various complex polysaccharides and other materials. This alternative pathway can also be initiated spontaneously by the cleavage of the thioester bond in C3 by a water molecule to form C3(H₂O). C3(H₂O) binds factor B, which allows factor D to cleave factor B to Ba and Bb. Bb remains associated with C3(H₂O) to form C3(H₂O)Bb complex, which acts as a C3 convertase and cleaves C3, resulting in C3a and C3b.

C3b formed either via this process or via the classical or lectin pathways binds to targets (e.g., on cell surfaces) and forms a complex with factor B, which is subsequently cleaved by factor D to form Bb, resulting in C3bBb, which is termed the alternative pathway (AP) C3 convertase. Binding of another molecule of C3b to the AP C3 convertase produces C3bBbC3b, which is the AP C5 convertase.

### Lectin

The lectin pathway is activated by polysaccharides with free-mannose group(s) and/or other sugars common to fungi and bacteria. The lectin complement pathway is initiated by binding of mannose-binding lectin (MBL) and MBL-associated serine protease (MASP) to carbohydrates. The MBL1 gene (known as LMANI in humans) encodes a type 1 integral membrane protein localized in the intermediate region between the endoplasmic reticulum and the Golgi. The MBL2 gene encodes the soluble mannose-binding protein found in serum. In the human lectin pathway, MASP1 and MASP2 are involved in proteolysis of C4 and C2, leading to C3 convertase, which lead to production of a C5 convertase as described above for the CP.

Lectin pathway (LP) proteins have been shown to function as activators and amplifiers of coagulation in hemostasis and thrombotic disease. MASP-1 and-2 are activated during blood clotting by activated platelets and generation of fibrin. MASP-1 and-2 are not only activated by fibrin clots but are also involved in fibrin clot generation. Specifically, MASP-1 has thrombin like specificity and is thus capable of catalyzing the formation of cross-linked fibrin. MASP-2 activates thrombin directly by cleaving prothrombin. Thus, a positive feedback loop exists in which MASP activation generates fibrin clots and clot formation activates MASPs. This is one way that complement activation is focused at sites of vascular injury.

### Complement and Diseases

The complement system is a network of fluid-phase and membrane-associated proteins designed to trigger, amplify, and regulate immunity and inflammation. Crosstalk between complement and cytokine networks can shape induced immune responses and outcomes. This interaction can be broadly classified into three categories: acute phase response, instruction of adaptive immunity, and sterile inflammation and regeneration. Several diseases, disorders, and/or conditions may result from, activate, and/or be impacted by one or more dysfunctions in one or more complement pathway, or complement pathway-associated components. For instance, as will be known to those of skill in the art, several autoimmune diseases have been shown to have changes in one or more complement-system related proteins. In some embodiments, technologies of the present disclosure can measure one or more targets to easily provide information about autoimmune diseases at one or more different stages (e.g., pre-diagnosis, post-diagnosis, during a flare or disease exacerbation, after treatment is started, etc.).

For example, systemic production of many complement proteins (C3, C4, C9, C4BP, MBL, Factor B and C1-INH) is controlled by cytokines (IL6 and IL1 family) released during the acute phase response (Gabay and Kushner, 1999, NEJM, 340: 448-54, the entirety of which is herein incorporated by reference). Complement activation amplifies the acute phase response by inducing IL1 and upregulating APR proteins, such as CRP (Szalai et al., 2000, Journal of immunology 165: 1030-35, the entirety of which is herein incorporated by reference). Thus, amplification between these two systems can rapidly follow an inflammatory trigger occur if regulatory mechanisms are absent or overwhelmed.

In addition to systemic production, local production of complement components also occurs and is under regulation by the action of cytokines. For example, expression of C3 by epithelial cells is enhanced by IL1B, IFN-γ and TNFα stimulation (Kulkarni et al., 2019, Am J Respir Cell Mol Biol 60: 144-157, the entirety of which is herein incorporated by reference) and these cytokines also upregulate C3, Factor D and Factor B production by endothelial cells (Raedler et al., 2009, Am J Transplant 9: 1784-1795, the entirety of which is herein incorporated by reference). Further, enhanced production of Factor H by endothelial cells is stimulated by IFN-γ (Brooimans et al., 1989, Journal of immunology 142: 2024-30, the entirety of which is herein incorporated by reference). As with systemic activation, reciprocal regulation of expression occurs, with complement activation fragments also modulating local cytokine response. C3a and C5a upregulate IL-8, IL-1B and RANTES and C5a decreases IL-6 expression by endothelial cells (Monsinjon et al., 2003, FASEB J 17: 1003-14, the entirety of which is herein incorporated by reference). The C5a/C5aR interaction suppresses TLR induced IL-6 and TNF production by macrophages while enhancing these responses in human monocytes (Seow et al., 2013, Journal of immunology 191: 4308-16, the entirety of which is herein incorporated by reference).

The early innate immune response is largely shaped by bidirectional crosstalk between the complement system and TLRs that appears to fine-tune a balance between inflammatory pathology and homeostatic immunity (Hajishengallis and Lambris, 2016, Immunol Rev 274: 233-44, the entirety of which is herein incorporated by reference). Microbial products that initiate TLR signaling, including LPS (TLR4), zymosan (TLR2/6) and CpG DNA (TLR9), also act as complement activators (Zhang et al., 2007, Blood 110: 228-36; Mangsbo et al., 2009, Journal of Immunology, 183: 6724-32; the entirety of each of which is herein incorporated by reference). This can lead to production of complement activation fragments that regulate TLR dependent responses through interaction with their respective receptors. In addition, TLR induced cytokines, such as IL-6, promote expression of complement components, including FB and the expression of C3aR/C5aR (Zhang et al., 2007, Blood 110: 228-36; Rittirsch et al., 2008, Nat Rev Immunol 8: 776-87; the entirety of each of which is herein incorporated by reference). These systems work both synergistically and antagonistically to regulate the induced response. For example, signaling through C3aR/C5aR with concurrent TLR stimulation drives synergistic increases in TNFα, IL-1B, IL-10 and IL-6 (Zhang et al., 2007). However, C1q acts antagonistically by inhibiting TLR7/9 induced IFNα production. Thus, TLRs can regulate expression of complement factors as well as expression and/or activation of complement receptors, which in turn can amplify or limit TLR-dependent responses.

Complement also instructs the immune system to respond appropriately to pathogens but also limit pathogenic inflammation (Ricklin et al., 2010, Nature Immunology 11: 785-97, the entirety of which is herein incorporated by reference). This impact begins with complement-TLR interactions that shape local inflammatory environment and extends to direct impact of complement receptor engagement on immune cells. For example, C1q inhibits IFNα production directly by interaction with LAIR1 on pDC, and indirectly through uptake of C1q-IC by monocytes. In human monocytes, C3a/C3aR interaction activates the inflammasome and IL-1B secretion and C3aR stimulated monocytes drive Th17 responses through enhanced IL-1B production (Asgari et al., 2013, Blood 122: 3473-81, the entirety of which is herein incorporated by reference). However, uptake of C1q opsonized apoptotic lymphocytes by LPS stimulated human macrophages increases expression of IFNα, IL-27 and IL-10, and inhibits inflammasome activation (cleavage of IL-1β) (Benoit et al., 2012, Journal of Immunology 188: 5682-93, the entirety of which is herein incorporated by reference). Conversely, inflammasome activation and release of IL-1B is additionally influenced by stimulation of complement activation including sublytic MAC and further primed by C5a in combination with TNF (Laudisi et al., 2013, Journal of Immunology 191: 1006-10; Espevik et al., 2014, Journal of Immunology 192: 2837-45, the entirety of each of which is herein incorporated by reference).

The complement system also has multiple roles in shaping the adaptive response, including regulating T cell responses. This includes directing the initiation phase, driving lineage commitment and regulating the contraction phase. Binding of locally produced C5a to its receptor, C5aR, expressed on APCs upregulates IL-12 production, which in turn directs T cell differentiation towards an IFN-γ producing phenotype (Lalli et al., 2007, Journal of Immunology, 179: 5793-802, the entirety of which is herein incorporated by reference). Locally produced C5a also augments CD8+ T cell IFN-γ and perforin expression (Raedler et al., 2009, Am J Transplant 9: 1784-1795, the entirety of which is herein incorporated by reference). The crosslinking of CD3 and the complement regulator CD46 on human CD4+ T cells in the presence of IL-2 leads to the induction of a regulatory T cell phenotype and release of IL-10 (Kemper and Atkinson, 2007, Nat Rev Immunol 7: 9-18; Cardone et al., 2010, Nature Immunology 11: 862-871; the entirety of each of which is herein incorporated by reference). CD46 generated regulatory T cells also express granzyme B and perforin, and display contact dependent cytotoxicity towards activated CD4+ and CD8+ T cells (Grossman et al., 2004, Immunity, 21: 589-601; Grossman et al., 2004, Blood 104: 2840-48; the entirety of each of which is herein incorporated by reference). Therefore, regulatory T cells generated through CD46 cross-linking have three mechanisms for suppressing the effector T cell response: secretion of IL-10, direct cytotoxicity through synthesis of granzyme B and perforin, and competition for IL-2 as a growth factor (Lalli et al., 2007, Journal of Immunology, 179: 5793-802, the entirety of which is herein incorporated by reference). Of note, CD46-induced regulatory T cells permit DC activation by dual secretion of GM-CSF and soluble CD40 (Barchet et al., 2006, Blood 107: 1497-1504, the entirety of each of which is herein incorporated by reference). Crosslinking of CD46 on human monocytes/macrophages suppresses IL-12 induction, providing a possible mechanism for measles virus induced immunosuppression (Karp et al., 1996, Science 273: 228-231, the entirety of which is herein incorporated by reference).

Complement not only promotes pathogen clearance but also has an essential role in homeostasis through resolution of inflammation by contributing to sterile inflammation, wound healing and regeneration. Apoptotic T cells rapidly lose CD46 from the cell surface and the removal of this protective signal promotes phagocytosis (Elward et al., 2005, The Journal of Biological Chemistry 280: 36342-354, the entirety of which is herein incorporated by reference). The interaction of iC3b on opsonized apoptotic cells with its receptor, CR3, on phagocytes promotes clearance and is accompanied by IL-12 downregulation to prevent undesirable inflammation during apoptotic cell clearance. Furthermore, if injury has occurred, complement contributes to homeostasis by promoting injury repair. This pro-repair role is evident in liver regeneration where C3a/C5a induced IL6 and TNFα signaling promotes hepatocyte growth and proliferation (Markiewski et al., 2006, Molecular Immunology, 43: 45-56, the entirety of which is herein incorporated by reference). Additionally, C3a and C5a induce expression of VEGF, which is required for tissue repair after injury (Nozaki et al., 2006, PNAS, 103: 2328-333, the entirety of which is herein incorporated by reference). Thus, complement contributes to the resolution of inflammation by participating in the non-inflammatory clearance of apoptotic cells and immune complexes and promoting repair of damaged tissue.

In parallel to the direct impact on the cell metabolic machinery, autocrine CD46 signaling also results in increased expression of interleukin 2Rα (IL-2Rα, CD25) and assembly of the high affinity IL-2 receptor (Liao W et al., Curr Opin Immunol., 2011; 23(5): 598-604; Liao W et al., Immunity, 2013; 38(1):13-25; West E E et al., Annu Rev Immunol. 2018; 36: 309-338; Merle N S et al., Br J Pharmacol., 2020; 1-17; each of which is herein incorporated by reference in its entirety).

Without being bound by any particular theory, complement activation in immediate and early post-trauma periods (e.g., after disease onset, after injury, etc.) can occur via several different mechanism, likely by use of one or more of any of the three complement pathways. Detecting such activation with appropriate reliability, reproducibility, sensitivity, specificity, accuracy, and/or speed (e.g., point-of-care, and regular monitoring intervals following injury) has remained a challenge in many contexts. The present disclosure provides technologies that allow for efficient, rapid, sensitive, specific, reliable, and/or accurate measurements of one or more targets (e.g., one or more complement proteins). With timely information, a clinician will be able to provide more appropriate and more rapid care which may, in some embodiments, prevent or improve certain outcomes (e.g., prevent continued damage through treatment, etc.) that would not have been achieved in the absence of technologies provided by the present disclosure.

As will be appreciated by those of skill in the art, several diseases, disorders, or conditions are characterized or impacted by changes in one or more complement proteins, or one or more complement-associated proteins. Despite the abundance of diseases, disorders, and conditions that are associated with one or more changes in one or more complement pathway proteins/associated proteins, there remain few to no satisfactory treatment options for many. Troublingly, there is a paucity of satisfactory diagnostic, prognostic, and monitoring methods in the field and, accordingly, lack of satisfactory treatment for many such diseases, disorders, and conditions.

As will be appreciated by those of skill in the art, target measurement in the complement system is known to be challenging for many reasons. For instance, some complement proteins are vulnerable to changes due to handling during and/or after collection of a sample, which may cause inaccurate measurements when such proteins are assayed. For instance, handling during or after collection can cause activation of certain complement proteins (C3), leading to cleavage fragment generation and/or degradation.

In addition, some complement proteins are high abundance targets and in previously available assays, measurement of such targets required excessive (e.g., several serial) dilution in order to be within a range that an assay could measure a target and also prevent inaccurate measurements due to prozone; however, techniques used to modify assays for high abundance targets risk preventing accurate measurements of low abundance targets.

Furthermore, endogenous levels of certain complement proteins or complement cleavage fragments can be very low in healthy patients, or in patients being treated with certain therapeutics, thus, amount of a given target may vary greatly between healthy subjects or a subject with one or more diseases, disorders, or conditions in which a complement pathway protein complement-pathway related protein is altered. In addition, as will be understood by those of skill in the art, inaccurate measurements of low abundance targets such as, e.g., certain complement cleavage fragments, can be problematic for various reasons including, but not limited to the understanding that small changes in certain cleavage fragments or low abundance targets can be physiologically relevant, but difficult to measure due to challenges described herein. The present disclosure provides technologies that overcome many of these challenges. Thus, in some embodiments, technologies of the present disclosure improve ability to accurately measure one or more targets in a sample (e.g., complement-related targets in a liquid sample).

For instance, by way of non-limiting example, in some embodiments, a subject may have an increase in complement C3, but previously available assays were only able to measure C3 in a given sample within a narrow range, despite highly abundant quantities of C3 in samples from patients. Furthermore, previously available assays that could measure C3 relied on serial dilutions to do so, which, as recognized by the present disclosure, can be a source of error in accurate measurements of C3 and/or a C3 therapeutic. For example, in the case of a C3 therapeutic, serial dilutions result in dissociation and falsely elevated levels of “free” C3 reported. Accordingly, the present disclosure provides, among other things, methods of diagnosis, monitoring, prevention, treatment of one or more complement-mediated diseases, disorders, conditions, and complications thereof.

Without being bound by any particular theory, the present disclosure contemplates that the ability to rapidly, accurately, sensitively, specifically, efficiently, and/or reliably measure one or more targets (e.g., complement-related targets) in a sample from a patient will improve outcomes in diagnostics, monitoring, and treatment. For example, the decision to administer one or more treatments, including dose and/or timing thereof, may change, depending upon the results of one or more measurements of particular targets (e.g., one or more complement-related targets). Complement-associated diseases, disorders, and/or conditions can be acute or chronic, and can be life-threatening/quality of life-impairing if not diagnosed, monitored and/or treated properly. Accordingly, it is an objective of the present disclosure to provide technologies that improve treatment of complement-associated diseases. In some embodiments, improving treatment is achieved by improving reliability, efficiency, sensitivity, specificity, accuracy, and/or how quickly one or more targets may be measured.

In some embodiments, a target may be MASP-2 and/or a disease may be COVID (i.e., SARS-COV-2). In some embodiments, MASP-2 may be altered in a patient suffering or at risk of suffering from COVID or complications related thereto. For instance, as will be known to those of skill in the art, in some embodiments, MASP-2 may bind directly to the SARS-COV-2 N protein. Additionally, MASP-2, C4d and C5b-9 deposition have been demonstrated in the pulmonary microvasculature of patients who succumbed to severe COVID-19. Accordingly, technologies of the present disclosure may be used to accurately, reliably, reproducibly, and quickly measure MASP-2 to diagnose, monitor, prevent and/or treat one or more symptoms related to COVID or risk of COVID infection or complications related thereto.

Another non-limiting example is vascular injury. For example, as will be known to those of skill in the art, following a vascular injury, complement factor H (FH) can protect a subject from excessive complement activation. For example, glycocalyx breakdown causes vascular endothelial cells to lose ability to bind FH, resulting in uncontrolled complement activation and damage. Mutations in FH are also considered strong genetic risk factors for disease such as age-related macular degeneration (AMD), aHUS, and complement 3 glomerulopathy (C3G). However, detecting such activation with appropriate reliability, reproducibility, accuracy, sensitivity, specificity and/or speed (e.g., point-of-care, and regular monitoring intervals following injury) has been a challenge and is paramount to successfully preventing or treating any sequelae of vascular injury. The present disclosure provides technologies that can overcome these challenges in order to provide measurements of one or more targets in an accurate, reliable, specific, reproducible, reliable, sensitive, and/or rapid, as well as easy-to-use method such as, for example, point-of-care, little to no pre-processing of a sample, etc. Accordingly, such technologies will improve treatment options for providers in contexts of vascular injuries.

In some embodiments, technologies provided by the present disclosure facilitate monitoring, diagnosis and treatment of diseases, disorders, and/or conditions that, until now, were difficult to accurately and reliably monitor, diagnose and/or treat. For instance, in some embodiments, a subject may have or be at risk of having or developing age-related macular degeneration (AMD), complement 3 glomerulopathy (C3G), Hematopoietic Stem Cell Transplant-Associated Thrombotic Microangiopathy (HSCT-TMA), Complement-Mediated Thrombotic Microangiopathy (CM-TMA), atypical hemolytic uremic syndrome (aHUS), thrombotic thrombocytopenia purpura (TTP), COVID19, lupus erythematosus, lupus nephritis, cytokine release syndrome, Alzheimer's Disease (AD), or combinations thereof. Thus, in contrast to previously available assays, technologies of the present disclosure will allow efficient, accurate, sensitive, reliable, specific and/or rapid results that will allow subjects to be properly diagnosed and treated for a variety of diseases, disorders, or conditions as contemplated herein.

### Targets

The present disclosure provides technologies for measuring one or more targets in one or more samples. In some embodiments, the present disclosure provides technologies that improve efficiency, sensitivity, accuracy, specificity, reliability and/or speed of target measurement, while also improving and increase the range across which a target may be detected as compared to previously available assays. In some embodiments, the present disclosure provides, for the first time technologies capable of measuring one or more complement pathway or complement-pathway-associated targets in a context of diagnosing, monitoring, preventing and/or treating one or more complement-associated diseases. In some such embodiments, diagnosis, monitoring, prevention, and treatment occurs in a point of care setting. As described herein one or more targets and diseases related thereto will be known to those of skill in the art, given the context.

As provided herein, the present disclosure recognizes that one source of a problem in sensitively, specifically, accurately, reliably, reproducibly, and/or rapidly measuring certain targets. For example, as described herein, in some embodiments, the present disclosure recognizes that diluting a sample to measure a target level may provide inaccurate measurements due to one or more pre-processing steps before a sample is measured. For instance, in some embodiments, offline dilution of a sample results in dissociation of a target from a target: therapeutic complex, causing inaccurate measurements.

Importantly, however, the present disclosure also provides the insight that certain targets may be high abundance or low abundance targets. In those cases, an inline dilution provides for accurate measurements of such targets, which was not possible in previously available assays.

In some embodiments, a target is a naturally occurring substance or agent (e.g., an endogenous protein). In some embodiments, a target is or comprises a biomarker. In some embodiments, a target is or comprises an analyte. In some embodiments, a target is or comprises a therapeutic. In some embodiments, a target is a complex, mixture or hybrid of a naturally occurring substance or agent and a non-naturally occurring substance or agent (e.g., a therapeutic). In some embodiments, a target is a combination of a biomarker and a therapeutic.

In some embodiments, a target is considered a high abundance target. For example, in some embodiments, a high abundance target is one that is highly concentrated in a sample as compared to one or more other targets or to a control level of the target. That is, in some embodiments, the level of a target is known to be at a high concentration either in a non-pathologic or pathologic state, such that standard assays (e.g. ELISAs) require dilutions in order to measure such a target. In some such embodiments, the present disclosure provides several advantages for measuring such high abundance targets without a need for more than minimal or, in some embodiments, with no “off-line” dilutions. That is, technologies provided by the present disclosure are able to manage a sample such that a high abundance target is efficiently, accurately, specifically, reliably, sensitively and/or rapidly measured. For example, in some embodiments, C3 is typically a high abundance target.

In some embodiments, a target is considered a low abundance target. For instance, in some embodiments, a low abundance target may be one that generally occurs at very low concentrations (e.g., pg/mL) or occurs in very low quantities as compared to one or more other targets such as a high abundance target. That is, in some embodiments, a level of a target is known to be at a low concentration either in a non-pathologic or pathologic state. In some embodiments, a target may be a low abundance target in a non-pathologic state and a high abundance target in a pathologic state or vice-versa. By way of non-limiting example, IL-6 is one such target that is generally considered low abundance target in non-pathologic states and may, in some embodiments, become a high abundance target in a setting of pathology.

In some embodiments, the present disclosure provides several advantages for measuring low abundance targets without a need for any significant pre-processing steps. For example, in some embodiments, the present disclosure provides an advantage of being able to measure one or more targets without having to perform significant offline dilutions on a sample prior to measuring; that is, in contrast to previously available assays, in some embodiments, the present disclosure provides an advantage of not having to perform several serial offline dilutions prior to measuring a target in a sample (e.g., as in an ELISA).

In some embodiments, the present disclosure provides technologies that need few to no pre-processing steps. For example, in some embodiments, technologies provided herein can perform inline normalization of a sample. In some such embodiments, for example, accurate measurements from urine samples are achieved by multiplexing creatinine measurement in assays of the present disclosure. Such inline normalization provides several advantages including but not limited to not having to dilute or manipulate a sample prior to measuring any given target. In addition, inline normalizations allow accurate measurements of one or more targets in heterogeneous samples such as urine, which can be differently concentrated even in a single patient. Thus, technologies provided by the present disclosure are able to efficiently, accurately, specifically, sensitively, reliably and/or rapidly measure targets, including high abundance and low abundance targets, in samples that would have previously required extensive pre-processing steps (e.g., dilutions or normalization interventions).

Importantly, and as will be appreciated by one of skill in the art, the present disclosure does not consider any given target to be categorically high abundance or low abundance target. Rather, as will be understood, given context, in some embodiments, a target may change from low abundance to high abundance such as when measured from a sample taken during a non-pathologic state as compared to a sample taken during a pathologic state. For example, without being bound by any particular theory, in some immune-mediated conditions (e.g., CM-TMA, aHUS, HSCT-TMA, cytokine release syndrome), a level of sC5b-9 may be considered high abundance as compared to a level of sC5b-9 in absence of such an immune-mediated condition.

In addition, in some embodiments, a type of sample may impact whether a target is high or low abundance. That is, in some embodiments, whether a sample is blood, plasma, urine, cerebrospinal fluid or another biological fluid may impact whether a target is high or low abundance. For instance, it is known that sC5b-9 was previously difficult or impossible to measure or even detect in urine. Technologies provided by the present disclosure are able to measure sC5b-9. Specifically, as described herein, technologies of the present disclosure not only allow detection but quantitation of a level of sC5b-9 in a sample of urine. In some embodiments, ability to measure targets such as sC5b-9 provides previously unavailable diagnostic, monitoring, and treatment methods to those in need thereof.

In some embodiments, a target is measured by comparison to a reference level or ratio is an established reference level or ratio for a target. In some embodiments, a target measurement is compared against a reference level or ratio obtained from a previous sample. In some embodiments, samples may be obtained from the same or different subjects. For instance, in some embodiments, a sample from a subject may be compared to a measurement of a sample taken from the same subject at different time. In some embodiments, the level or ratio of the one or more indicator(s) (e.g., complement proteins) is compared against a reference level or ratio which was obtained from a different subject or population of subject (e.g., a composite score), or a normal reference range.

In some embodiments, a target may be a low abundance target in one state (e.g., presence or absence of a disease, disorder, or condition) and a high abundance target in another state (e.g., absence or presence of the same disease, disorder or condition).

In some embodiments, a target that is not detected may be considered absent. In some such embodiments, an absent target may not be present in a sample or may be present below a LLOQ of an assay as described herein. In some embodiments, a target may be absent in one sample from a subject and present in another sample from the same subject. For example, in some embodiments, a target may be absent in blood from a subject but present in urine or vice-versa. In some embodiments, a target may be present in more than one sample from the same subject, e.g., present in both blood and urine.

### Exemplary Targets

### Complement C3

Complement component C3 is useful as a general alert biomarker that the body of an organism is responding to some form of physiological crisis, such as injury, infection, or other disease process. In some embodiments, a level of C3 increases (relative to a reference level of C3) in the context of one or more diseases, disorders or conditions. In some embodiments, a level of C3 decreases (relative to a reference level of C3) in the context of one or more diseases, disorders or conditions. In some embodiments, a level of C3 does not change (relative to a reference level of C3) in the context of one or more diseases, disorders or conditions, but levels of other complement proteins or components thereof change. For instance, by way of non-limiting example, in some embodiments, a level of C3 does not change, but a level of iC3b may increase or decrease.

In some embodiments, intact C3 may be measured in a sample in a range of 0.025-5 mg/mL.

In some embodiments, a “normal” intact C3 level in a sample (e.g., bodily fluid) falls within a range with a lower boundary and an upper boundary that is higher than the lower boundary. In some embodiments, a “normal” intact C3 level in a sample falls within a range of 0.025-5 mg/mL. In some embodiments, the lower boundary may be at least about 25 μg/mL, 30 μg/mL, 35 μg/mL, 40 μg/mL, 45 μg/mL, 50 μg/mL, 55 μg/mL, 60 μg/mL, 65 μg/mL, 70 μg/mL, 75 μg/mL, 80 μg/mL, 85 μg/mL, 90 μg/mL, 95 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, 250 μg/mL, 260 μg/mL, 270 μg/mL, 280 μg/mL, 290 μg/mL, 300 μg/mL, 310 μg/mL, 320 μg/mL, 330 μg/mL, 340 μg/mL, 350 μg/mL, 360 μg/mL, 370 μg/mL, 380 μg/mL, 390 μg/mL, 400 μg/mL, 410 μg/mL, 420 μg/mL, 430 μg/mL, 440 μg/mL, 450 μg/mL, 460 μg/mL, 470 μg/mL, 480 μg/mL, 490 μg/mL, 500 μg/mL, 510 μg/mL, 520 μg/mL, 530 μg/mL, 540 μg/mL, 550 μg/mL, 560 μg/mL, 570 μg/mL, 580 μg/mL, 590 μg/mL, 600 μg/mL, 610 μg/mL, 620 μg/mL, 630 μg/mL, 640 μg/mL, 650 μg/mL, 660 μg/mL, 670 μg/mL, 680 μg/mL, 690 μg/mL, 700 μg/mL, 710 μg/mL, 720 μg/mL, 730 μg/mL, 740 μg/mL, 750 μg/mL, 760 μg/mL, 770 μg/mL, 780 μg/mL, 790 μg/mL, 800 μg/mL, 810 μg/mL, 820 μg/mL, 830 μg/mL, 840 μg/mL, 850 μg/mL, 860 μg/mL, 870 μg/mL, 880 μg/mL, 890 μg/mL, 900 μg/mL, 910 μg/mL, 920 μg/mL, 930 μg/mL, 940 μg/mL, 950 μg/mL, 960 μg/mL, 970 μg/mL, 980 μg/mL, 990 μg/mL, 1000 μg/mL, 1100 μg/mL, 1200 μg/mL, 1300 μg/mL, 1400 μg/mL, 1500 μg/mL, 1600 μg/mL, 1700 μg/mL, 1800 μg/mL, 1900 μg/mL, 2000 μg/mL, 2100 μg/mL, 2200 μg/mL, 2300 μg/mL, 2400 μg/mL, 2500 μg/mL, 2600 μg/mL, 2700 μg/mL, 2800 μg/mL, 2900 μg/mL, 3000 μg/mL, 3100 μg/mL, 3200 μg/mL, 3300 μg/mL, 3400 μg/mL, 3500 μg/mL, 3600 μg/mL, 3700 μg/mL, 3800 μg/mL, 3900 μg/mL, 4000 μg/mL, 4100 μg/mL, 4200 μg/mL, 4300 μg/mL, 4400 μg/mL, 4500 μg/mL, 4600 μg/mL, 4700 μg/mL, 4800 μg/mL, 4900 μg/mL or more. In some embodiments, the upper boundary may be at least about 5000 μg/mL, 4950 μg/mL, 4850 μg/mL, 4750 μg/mL, 4650 μg/mL, 4550 μg/mL, 4450 μg/mL, 4350 μg/mL, 4250 μg/mL, 4250 μg/mL, 4050 μg/mL, 3950 μg/mL, 3850 μg/mL, 3750 μg/mL, 3650 μg/mL, 3550 μg/mL, 3450 μg/mL, 3350 μg/mL, 3250 μg/mL, 3150 μg/mL, 3050 μg/mL, 2950 μg/mL, 2850 μg/mL, 2750 μg/mL, 2650 μg/mL, 2550 μg/mL, 2450 μg/mL, 2350 μg/mL, 2250 μg/mL, 2150 μg/mL, 2050 μg/mL, 1950 μg/mL, 1850 μg/mL, 1750 μg/mL, 1650 μg/mL, 1550 μg/mL, 1450 μg/mL, 1350 μg/mL, 1250 μg/mL, 1150 μg/mL, 1050 μg/mL, 950 μg/mL, 850 μg/mL, 750 μg/mL, 650 μg/mL, 550 μg/mL, 450 μg/mL, 350 μg/mL, 250 μg/mL, 150 μg/mL, 100 μg/mL, 95 μg/mL, 90 μg/mL, 85 μg/mL, 80 μg/mL, 75 μg/mL, 70 μg/mL, 65 μg/mL, 60 μg/mL, 55 μg/mL, 50 μg/mL, 45 μg/mL, 40 μg/mL, 35 μg/mL, 30 μg/mL, 25 μg/mL, 20 μg/mL, 19 μg/mL, 18 μg/mL, 17 μg/mL, 16 μg/mL, 15 μg/mL, 14 μg/mL, 13 μg/mL, 12 μg/mL, 11 μg/mL, 10 μg/mL, 9 μg/mL, 8 μg/mL, 7 μg/mL, 6 μg/mL, 5 μg/mL, 4.5 μg/mL, 4 μg/mL, 3.5 μg/mL, 3 μg/mL, 2.5 μg/mL, 2 μg/mL, 1.9 μg/mL, 1.8 μg/mL, 1.7 μg/mL, 1.6 μg/mL, 1.5 μg/mL, 1.4 μg/mL, 1.3 μg/mL, 1.2 μg/mL, 1.1 μg/mL, 1.0 μg/mL, 0.9 μg/mL, 0.8 μg/mL, 0.7 μg/mL, 0.6 μg/mL or less. In some embodiments a “normal” level of intact C3 falls within the range of . . . 025 μg/mL-5 mg/mL; in other embodiments a “normal” level of intact C3 falls within the range of 100 μg/mL-4 mg/mL; in other embodiments a “normal” level of intact C3 falls within the range of 100 μg/mL-3 mg/mL; in other embodiments a “normal” level of intact C3 falls within the range of 200 μg/mL-2 mg/mL; in other embodiments a “normal” level of intact C3 falls within the range of 500 μg/mL-1.5 mg/mL; in other embodiments a “normal” level of intact C3 falls within the range of 500 μg/mL-1.0 mg/mL; in other embodiments a “normal” level of intact C3 falls within the range of 500 μg/mL-750 μg/mL.

In some embodiments, intact C3 is detected using a non-cross-reactive agent such as a non-cross-reactive antibody or other C3 binding agent.

### C3a

Complement component C3a is one of the cleavage products of C3. As will be known to those of skill in the art given context, C3a can have a variety of proinflammatory and anti-inflammatory effects. C3a is a volatile biomarker with a very short half-life of approximately two minutes. Accordingly, there is an unmet need for methods that can rapidly, accurately, sensitively, specifically, and reliably measure C3a in a sample. In some embodiments, a level of C3a increases (relative to a reference level of C3a) in the context of one or more diseases, disorders or conditions. In some embodiments, a level of C3a decreases (relative to a reference level of C3a) in the context of one or more diseases, disorders or conditions. In some embodiments, a level of C3a does not change (relative to a reference level of C3a) in the context of one or more diseases, disorders or conditions, but levels of other complement proteins or components thereof change. In some embodiments, a level of C3a increase or decrease while an overall level of C3 does not change.

In some embodiments, C3a may be measured in a sample in a range of 1-3000 ng/mL.

In some embodiments, a “normal” C3a level in a sample (e.g., bodily fluid) falls within a range with a lower boundary and an upper boundary that is higher than the lower boundary. In some embodiments, a “normal” C3a level in a sample falls within a range of 1-3000 ng/mL In some embodiments, the lower boundary may be at least about 1 ng/ml, 2 ng/ml, 3 ng/ml, 4 ng/mL, 5 ng/mL, 6 ng/ml, 7 ng/ml, 8 ng/mL, 9 ng/mL, 10 ng/ml, 11 ng/mL, 12 ng/ml, 13 ng/mL, 14 ng/mL, 15 ng/ml, 16 ng/ml, 17 ng/mL, 18 ng/ml, 19 ng/ml, 20 ng/mL, 25 ng/mL, 30 ng/mL, 35 ng/mL, 40 ng/mL, 45 ng/mL, 50 ng/mL, 55 ng/mL, 60 ng/mL, 65 ng/mL, 70 ng/mL, 75 ng/mL, 80 ng/mL, 85 ng/mL, 90 ng/mL, 95 ng/mL, 100 ng/mL, 150 ng/mL, 200 ng/mL, 250 ng/mL, 300 ng/ml, 350 ng/mL, 400 ng/mL, 450 ng/mL, 500 ng/mL, 550 ng/mL, 600 ng/mL, 650 ng/mL, 700 ng/mL, 750 ng/ml, 800 ng/ml, 850 ng/ml, 900 ng/ml, 950 ng/mL, 1000 ng/mL, 1100 ng/mL, 1200 ng/mL, 1300 ng/ml, 1400 ng/ml, 1500 ng/mL, 1600 ng/mL, 1700 ng/mL, 1800 ng/mL, 1900 ng/ml, 2000 ng/ml, 2100 ng/mL, 2200 ng/mL, 2300 ng/mL, 2400 ng/mL, 2500 ng/mL, 2600 ng/mL, 2700 ng/mL, 2800 ng/ml, 2900 ng/mL, 3000 ng/ml or more. In some embodiments, the upper boundary may be at least about 3000 ng/mL, 2950 ng/mL, 2850 ng/mL, 2750 ng/mL, 2650 ng/mL, 2550 ng/ml, 2450 ng/ml, 2350 ng/mL, 2250 ng/mL, 2150 ng/mL, 2050 ng/mL, 1950 ng/mL, 1850 ng/ml, 1750 ng/mL, 1650 ng/mL, 1550 ng/mL, 1450 ng/mL, 1350 ng/mL, 1250 ng/mL, 1150 ng/mL, 1050 ng/ml, 1000 ng/ml, 975 ng/mL, 925 ng/mL, 875 ng/mL, 825 ng/mL, 775 ng/mL, 725 ng/mL, 675 ng/ml, 625 ng/mL, 575 ng/mL, 525 ng/mL, 475 ng/mL, 425 ng/mL, 375 ng/ml, 325 ng/ml, 275 ng/ml, 225 ng/ml, 175 ng/ml, 125 ng/mL, 100 ng/ml, 95 ng/mL, 90 ng/mL, 85 ng/ml, 80 ng/mL, 75 ng/mL, 70 ng/mL, 65 ng/mL, 60 ng/mL, 55 ng/mL, 50 ng/mL, 45 ng/mL, 40 ng/ml, 35 ng/mL, 30 ng/mL, 25 ng/mL, 20 ng/ml, 19 ng/mL, 18 ng/mL, 17 ng/mL, 16 ng/ml, 15 ng/ml, 14 ng/ml, 13 ng/mL, 12 ng/mL, 11 ng/mL, 10 ng/mL, 9 ng/mL, or less. In some embodiments a “normal” level of intact C3 falls within the range of 1-3000 ng/mL; in other embodiments a “normal” level of intact C3 falls within the range of 1-2500 ng/mL; in other embodiments a “normal” level of intact C3 falls within the range of 1-2000 ng/ml; in other embodiments a “normal” level of intact C3 falls within the range of 1-1500 ng/ml; in other embodiments a “normal” level of intact C3 falls within the range of 1-1000 ng/ml; in other embodiments a “normal” level of intact C3 falls within the range of 1-750 ng/ml; in other embodiments a “normal” level of intact C3 falls within the range of 1-500 ng/ml; in other embodiments a “normal” level of intact C3 falls within the range of 2.5 ng/ml-300 ng/ml; in other embodiments a “normal” level of intact C3 falls within the range of 4.5-200 ng/ml; in other embodiments a “normal” level of intact C3 falls within the range of 25 ng/ml-100 ng/ml; in other embodiments a “normal” level of intact C3 falls within the range of 50 ng/mL-100 ng/ml.

In some embodiments, C3a is detected using a non-cross reactive binding agent.

iC3b

iC3b protein is a breakdown product of C3, as shown in FIG. 1. In some embodiments, iC3b can be a valuable marker of inflammatory response. Importantly, in some embodiments, iC3b has a half-life of 30 to 90 minutes, serving as a less volatile (compared to C3a), but still rapidly responsive biomarker. However, iC3b is generally present at much lower levels than intact or total C3 in patient samples. Thus, even a small degree of cross-talk (for example 1%) between intact C3 protein and the iC3b-specific detecting agents is very likely to produce a false positive iC3b signal at a level twice that of normal circulating iC3b. Accordingly, the present disclosure provides technologies that overcome this and other challenges to improve efficiency, accuracy, sensitivity, specificity, reliability and/or speed of measurements of iC3b.

In some embodiments, iC3b in a sample may be elevated in comparison to a control, indicating C3 is activated and has been further split into its activation product, iC3b. In some embodiments, the level or concentration of intact C3 in a sample is decreased in comparison to a control, indicating that intact C3 has been converted to its breakdown or activation products and is hence depleted in the individual.

In some embodiments, iC3b may be measured in a sample in a range of 0.2-50 μg/mL.

In some embodiments, a “normal” iC3b level in a sample (e.g., bodily fluid) falls within a range with a lower boundary and an upper boundary that is higher than the lower boundary. In some embodiments, a “normal” iC3b level in a sample falls within a range of 0.2-50 μg/mL. In some embodiments, the lower boundary may be at least about 0.2 μg/mL, 0.3 μg/mL, 0.4 μg/mL, 0.5 μg/mL, 0.6 μg/mL, 0.7 μg/mL, 0.8 μg/mL, 0.9 μg/mL, 1 μg/mL, 2 μg/mL, 3 μg/mL, 4 μg/mL, 5 μg/mL, 6 μg/mL, 7 μg/mL, 8 μg/mL, 9 μg/mL, 10 μg/mL, 11 μg/mL, 12 μg/mL, 13 μg/mL, 14 μg/mL, 15 μg/mL, 16 μg/mL, 17 μg/mL, 18 μg/mL, 19 μg/mL, 20 μg/mL, 21 μg/mL, 22 μg/mL, 23 μg/mL, 24 μg/mL, 25 μg/mL, 26 μg/mL, 27 μg/mL, 28 μg/mL, 29 μg/mL, 30 μg/mL, 31 μg/mL, 32 μg/mL, 33 μg/mL, 34 μg/mL, 35 μg/mL, 36 μg/mL, 37 μg/mL, 38 μg/mL, 39 μg/mL, 40 μg/mL, 41 μg/mL, 42 μg/mL, 43 μg/mL, 44 μg/mL, 45 μg/mL, 46 μg/mL, 47 μg/mL, 48 μg/mL, 49 μg/mL or more. In some embodiments, the upper boundary may be at least about 50 μg/mL, 45 μg/mL, 40 μg/mL, 35 μg/mL, 30 μg/mL, 25 μg/mL, 20 μg/mL, 19 μg/mL, 18 μg/mL, 17 μg/mL, 16 μg/mL, 15 μg/mL, 14 μg/mL, 13 μg/mL, 12 μg/mL, 11 μg/mL, 10 μg/mL, 9 μg/mL, 8 μg/mL, 7 μg/mL, 6 μg/mL, 5 μg/mL, 4.5 μg/mL, 4 μg/mL, 3.5 μg/mL, 3 μg/mL, 2.5 μg/mL, 2 μg/mL, 1.9 μg/mL, 1.8 μg/mL, 1.7 μg/mL, 1.6 μg/mL, 1.5 g/mL, 1.4 μg/mL, 1.3 μg/mL, 1.2 μg/mL, 1.1 μg/mL, 1.0 μg/mL, 0.9 μg/mL, 0.8 μg/mL, 0.7 μg/mL, 0.6 μg/mL, 0.5 μg/mL, 0.4 μg/mL, 0.3 μg/mL, 0.2 μg/mL, or less. In some embodiments a “normal” level of iC3b falls within the range of 0.2 μg/mL-50 μg/mL; in other embodiments a “normal” level of iC3b falls within the range of 0.5 μg/mL mL-40 μg/mL; in other embodiments a “normal” level of iC3b falls within the range of 1 μg/mL/mL-35 μg/mL; in other embodiments a “normal” level of iC3b falls within the range of 5 μg/mL-30 μg/mL; in other embodiments a “normal” level of iC3b falls within the range of 7.5 μg/mL-25 μg/mL; in other embodiments a “normal” level of iC3b falls within the range of 10 μg/mL-20 μg/mL; in other embodiments a “normal” level of iC3b falls within the range of 12.5 μg/mL-17.5 μg/mL.

In some embodiments, iC3b is detected using a non-cross reactive binding agent such as, for example, an antibody characterized in that a 1 μg/μl solution of intact C3 produces signal equivalent to less than about 1 ng/ml of iC3b. In some embodiments, the non-cross-reactive antibody is selected from the group consisting of A209, MCA2607, and HM2199.

### C4

Like C3, complement component C4 is one of the most commonly measured complement proteins and is known to play roles in immunity (including autoimmunity) and tolerance. C4 is involved in all three complement pathways (i.e., classical, alternative, and lectin). As will be known to those of skill in the art, in some contexts C4 is cleaved into C4a and C4b (e.g., by C1s), with C4b being of higher molecular weight than C4a. In some embodiments, C4b may interact with complement protein 2 (C2), which itself may be cleaved into two components (e.g., by C1s) and may, in some embodiments, interact further, including, e.g., with C3.

In some embodiments, abnormal levels of intact C4 may exist in several different diseases, disorders or conditions. In some such embodiments, intact C4 levels may be increased or decreased relative to a control level. For example, in some embodiments, a level of intact C4 may be elevated or increased (i.e., as compared to a control level) during or after an acute infection or injury whereas in a chronic autoimmune condition (e.g., lupus, e.g., SLE, lupus nephritis, etc.), intact C4 levels may be decreased. In some embodiments, levels of intact C4 are decreased due to inherited or acquired diseases, disorders, or conditions. In some such embodiments, a genetic deficiency may result in reduced levels of C4 relative to a control level.

In some embodiments, intact C4 may be measured in a sample in a range of 0.05-1.0 mg/mL.

In some embodiments, a “normal” intact C4 level in a sample (e.g., bodily fluid) falls within a range with a lower boundary and an upper boundary that is higher than the lower boundary. In some embodiments, a “normal” intact C4 level in a sample falls within a range of 0.05-1.0 mg/mL. In some embodiments, the lower boundary may be at least about 50 μg/mL, 55 μg/mL, 60 μg/mL, 65 μg/mL, 70 μg/mL, 75 μg/mL, 80 μg/mL, 85 μg/mL, 90 μg/mL, 95 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, 250 g/mL, 260 μg/mL, 270 μg/mL, 280 μg/mL, 290 μg/mL, 300 μg/mL, 310 μg/mL, 320 μg/mL, 330 μg/mL, 340 μg/mL, 350 μg/mL, 360 μg/mL, 370 μg/mL, 380 g/mL, 390 μg/mL, 400 μg/mL, 410 g/mL, 420 μg/mL, 430 μg/mL, 440 μg/mL, 450 μg/mL, 460 μg/mL, 470 μg/mL, 480 uμ/mL, 490 μg/mL, 500 μg/mL, 510 μg/mL, 520 μg/mL, 530 μg/mL, 540 μg/mL, 550 μg/mL, 560 μg/mL, 570 μg/mL, 580 μg/mL, 590 μg/mL, 600 μg/mL, 610 μg/mL, 620 μg/mL, 630 μg/mL, 640 μg/mL, 650 μg/mL, 660 μg/mL, 670 μg/mL, 680 μg/mL, 690 μg/mL, 700 μg/mL, 710 μg/mL, 720 g/mL, 730 μg/mL, 740 μg/mL, 750 μg/mL, 760 μg/mL, 770 μg/mL, 780 μg/mL, 790 μg/mL, 800 μg/mL, 810 μg/mL, 820 μg/mL, 830 g/mL, 840 g/mL, 850 μg/mL, 860 μg/mL, 870 μg/mL, 880 μg/mL, 890 μg/mL, 900 μg/mL, 910 μg/mL, 920 μg/mL, 930 μg/mL, 940 μg/mL, 950 μg/mL, 960 μg/mL, 970 μg/mL, 980 μg/mL, 990 μg/mL or more. In some embodiments, the upper boundary may be at least about 1000 μg/mL, 950 μg/mL, 900 μg/mL, 850 μg/mL, 800 μg/mL, 750 μg/mL, 700 g/mL, 650 μg/mL, 600 μg/mL, 550 μg/mL, 500 μg/mL, 450 μg/mL, 400 μg/mL, 350 g/mL, 300 μg/mL, 250 μg/mL, 200 μg/mL, 150 μg/mL, 100 μg/mL, 90 μg/mL, 80 μg/mL, 70 μg/mL, 60 μg/mL, 50 μg/mL or less. In some embodiments a “normal” level of intact C4 falls within the range of 0.05-1 mg/mL; in other embodiments a “normal” level of intact C4 falls within the range of 50 μg/mL-900 μg/mL; in other embodiments a “normal” level of intact C4 falls within the range of 50 g/mL-800 μg/mL; in other embodiments a “normal” level of intact C4 falls within the range of 50 g/mL-700 μg/mL; in other embodiments a “normal” level of intact C4 falls within the range of 50 μg/mL-600 μg/mL; in other embodiments a “normal” level of intact C4 falls within the range of 50 μg/mL-500 μg/mL; in other embodiments a “normal” level of intact C4 falls within the range of 50 μg/mL-400 μg/mL; in other embodiments a “normal” level of intact C4 falls within the range of 50 μg/mL-300 μg/mL; in other embodiments a “normal” level of intact C4 falls within the range of 50 μg/mL-200 μg/mL; in other embodiments a “normal” level of intact C4 falls within the range of 50 μg/mL-100 μg/mL.

In some embodiments, intact C4 is detected using a non-cross reactive binding agent such as an antibody characterized in that it recognizes intact C4 and does not cross react, including with C4d.

### C5

Complement component 5 (C5) is important in inflammatory processes as well as several other cellular processes and events. As seen in FIG. 1, C5 convertase generated by any of the three complement pathways cleaves intact C5 to C5a and C5b. C5b can then bind to C6, C7, and C8, which is known to catalyze polymerization of C9 to form the C5b-9 membrane attack complex (MAC). C5a can be pro-inflammatory, and MAC (catalyzed by C5b) is involved in cellular lysis. Accordingly, in some embodiments, cleavage of C5 into C5a and C5b can be problematic. For example, in some embodiments, cleavage of C5 can be involved in pathological outcomes (e.g., one or more diseases, disorders, or conditions, e.g., aHUS). There are known monoclonal antibody therapies (e.g., anti-C5 antibodies) which can reduce or prevent C5 cleavage. Troublingly, accurately measuring therapeutic impact of such antibodies using biomarkers was almost impossible prior to the technologies provided by the present disclosure. For instance, the present disclosure recognizes that if a subject has been or is receiving treatment with a therapy that prevents or inhibits C5 cleavage (e.g., an anti-C5 antibody), a provider may measure an amount of free C5 in a sample (e.g., a blood sample) from a patient in order to titrate and/or monitor treatment. Previously available assays that measure C5 require several serial dilutions of a liquid sample (e.g., a blood sample). Problematically, however, dilutions of such a sample can result in dissociation of a therapeutic agent/target complex (dissociation of an anti-C5 antibody from C5). If dissociation of a therapeutic agent/target complex occurs, measurements of free C5 will then be artificially elevated and a provider may change a treatment that a subject is receiving (e.g., increasing an amount of an anti-C5 antibody) based on such measurements when, in fact, changes may not have been warranted. The present disclosure recognizes this source of a problem and also provides solutions with technologies that achieve efficient, sensitive, accurate, specific, reliable and/or rapid assays to measure C5 without a need to dilute a sample as in previously available assays, thus avoiding risk of erroneously reported C5 measurements.

In some embodiments, C5 may be measured in a sample in a range of 0.001-1000 μg/mL.

In some embodiments, a “normal” C5 level in a sample (e.g., bodily fluid) falls within a range with a lower boundary and an upper boundary that is higher than the lower boundary. In some embodiments, a “normal” C5 level in a sample falls within a range of 0.001-100 μg/mL. In some embodiments, the lower boundary may be at least about 0.001 μg/mL, 0.002 μg/mL, 0.003 μg/mL, 0.004 μg/mL, 0.005 μg/mL, 0.006 μg/mL, 0.007 μg/mL, 0.008 μg/mL, 0.009 μg/mL, 0.010 μg/mL, 0.015 μg/mL, 0.02 μg/mL, 0.025 μg/mL, 0.030 μg/mL, 0.035 μg/mL, 0.040 μg/mL, 0.045 μg/mL, 0.050 μg/mL, 0.055 μg/mL, 0.060 μg/mL, 0.065 μg/mL, 0.070 μg/mL, 0.075 μg/mL, 0.080 μg/mL, 0.085 μg/mL, 0.090 μg/mL, 0.095 μg/mL, 0.10 μg/mL, 0.15 μg/mL, 0.2 μg/mL, 0.25 μg/mL, 0.3 μg/mL, 0.35 μg/mL, 0.4 μg/mL, 0.45 μg/mL, 0.5 μg/mL, 0.55 μg/mL, 0.60 g/mL, 0.65 μg/mL, 0.70 μg/mL, 0.75 μg/mL, 0.8 μg/mL, 0.85 μg/mL, 0.9 μg/mL, 1 μg/mL, 2 μg/mL, 3 μg/mL, 4 μg/mL, 5 μg/mL, 6 μg/mL, 7 μg/mL, 8 μg/mL, 9 g/mL, 10 μg/mL, 15 μg/mL, 20 μg/mL, 25 μg/mL, 30 μg/mL, 35 μg/mL, 40 μg/mL, 45 μg/mL, 50 μg/mL, 55 μg/mL, 60 μg/mL, 65 μg/mL, 70 μg/mL, 75 μg/mL, 80 μg/mL, 85 μg/mL, 90 μg/mL, 95 μg/mL, 100 μg/mL, 150 μg/mL, 200 μg/mL, 250 g/mL, 300 μg/mL, 350 g/mL, 400 μg/mL, 450 μg/mL, 500 μg/mL, 550 μg/mL, 600 μg/mL, 650 μg/mL, 700 μg/mL, 750 μg/mL, 800 μg/mL, 850 g/mL, 900 μg/mL, 950 μg/mL, 1000 μg/mL or more. In some embodiments, the upper boundary may be at least about 1000 g/mL, 975 μg/mL, 925 μg/mL, 875 μg/mL, 825 μg/mL, 775 μg/mL, 725 μg/mL, 675 μg/mL, 625 μg/mL, 575 μg/mL, 525 μg/mL, 475 μg/mL, 425 μg/mL, 375 μg/mL, 325 μg/mL, 275 μg/mL, 225 μg/mL, 175 μg/mL, 125 μg/mL, 100 μg/mL, 95 μg/mL, 90 μg/mL, 85 μg/mL, 80 μg/mL, 75 μg/mL, 70 μg/mL, 65 μg/mL, 60 μg/mL, 55 μg/mL, 50 μg/mL, 45 μg/mL, 40 μg/mL, 35 μg/mL, 30 μg/mL, 25 μg/mL, 20 μg/mL, 15 μg/mL, 10 μg/mL, 9 μg/mL, 8 μg/mL, 7 μg/mL, 6 μg/mL, 5 μg/mL, 4 μg/mL, 3 μg/mL, 2 μg/mL, 1 μg/mL, 0.9 μg/mL, 0.8 μg/mL, 0.7 μg/mL, 0.6 μg/mL, 0.5 μg/mL, 0.4 μg/mL, 0.3 μg/mL, 0.2 μg/mL, 0.1 μg/mL, 0.09 μg/mL, 0.08 μg/mL, 0.07 μg/mL, 0.06 μg/mL, 0.05 μg/mL, 0.04 μg/mL, 0.03 μg/mL, 0.02 μg/mL, 0.01 μg/mL, 0.009 μg/mL, 0.008 μg/mL, 0.007 μg/mL, 0.006 μg/mL, 0.005 μg/mL, 0.004 μg/mL, 0.003 μg/mL, 0.002 μg/mL, or less. In some embodiments a “normal” level of C5 falls within the range of 0.001-1000 μg/mL; in other embodiments a “normal” level of C5 falls within the range of 0.001-750 μg/mL; in other embodiments a “normal” level of C5 falls within the range of 0.001-500 μg/mL; in other embodiments a “normal” level of C5 falls within the range of 0.001 μg/mL-250 μg/mL; in other embodiments a “normal” level of C5 falls within the range of 0.01 μg/mL-100 g/mL; in other embodiments a “normal” level of C5 falls within the range of 0.05 μg/mL-50 μg/mL; in other embodiments a “normal” level of C5 falls within the range of 0.1 μg/mL-10 μg/mL; in other embodiments a “normal” level of C5 falls within the range of 0.1 μg/mL-1 μg/mL.

In some embodiments, C5 is detected using a non-cross reactive binding agent such as an antibody characterized in that it does not cross react including with C5b or C5 complexed with a therapeutic agent.

sC5b-9

Following C5 activation, the terminal complement pathway cascade (TP) assembles complement components C5b, C6, C7, C8, and C9 to form C5b-9, a terminal complement complex. C5b-9 can insert into and damage cellular membranes or remain in an aqueous phase where it is measurable in its soluble form, sC5b-9. In some embodiments, a level of sC5b-9 in a sample may be elevated in the context of one or more diseases, disorders, or conditions. Without being bound by any particular theory, in some embodiments sC5b-9 in urine may be associated with status of C5 activation (e.g., inhibition of C5 activation). In contrast to the technologies provided by the present disclosure, previously available assays were not able to detect sC5b-9 below a threshold that was often too high for an amount of sC5b-9 present in many samples. Furthermore, measuring sC5b-9 in fluids other than blood (e.g., urine) was problematic because levels of sC5b-9 in urine are often significantly lower than in blood or plasma. Lower levels of sC5b-9 in urine as compared to other samples such as blood or plasma results in previously available assays often reporting false negatives when measuring sC5b-9 due to inability to reach the LLOQ that is achieved with technologies of the present disclosure. Thus, technologies described herein overcome challenges associated with sC5b-9 including by increasing range of detection (lower LLOQ and higher ULOQ) and improving efficiency, sensitivity, specificity, accuracy, reliability and/or speed of sC5b-9 measurements across a variety of samples, including urine. Accordingly, in some embodiments and in contrast to previously available assays, measuring sC5b-9 in one or more samples, including in urine may be used to determine status of C5 activation.

As described herein, among other things, the present disclosure provides insight that measurement of sC5b-9 in urine requires different conditions and normalization steps than with a sample such as blood. In addition, the present disclosure recognizes that volume of urine may drastically impact measurements of sC5b-9. Accordingly, in some embodiments, assays of the present disclosure also measure creatinine using the same immunoassay device as sC5b-9 in order to normalize measurements to account for changes in urine volumes and concentrations. In some embodiments, creatinine and sC5b-9 may be measured on the same test strip. In some embodiments, creatinine and sC5b-9 may be measured on different test strips (within the same immunoassay device).

In some embodiments, sC5b-9 may be measured in a sample in a range of 0.001-1000 μg/mL. In some embodiments, sC5b-9 in blood or plasma may be measured in a range of 50-10,000 ng/mL. In some embodiments, sC5b-9 in urine may be measured in a range of 1-100,000 ng/mL. In some embodiments, where a sample is or comprises urine, a level of creatinine may be measured in a range of 6.2-8000 μg/mL.

In some embodiments, a “normal” sC5b-9 level in a sample (e.g., bodily fluid) falls within a range with a lower boundary and an upper boundary that is higher than the lower boundary. In some embodiments, a “normal” sC5b-9 level in a sample falls within a range of 0.001-1000 μg/mL. In some embodiments, the lower boundary may be at least about 0.001 μg/mL, 0.002 μg/mL, 0.003 μg/mL, 0.004 μg/mL, 0.005 μg/mL, 0.006 μg/mL, 0.007 μg/mL, 0.008 g/mL, 0.009 μg/mL, 0.010 μg/mL, 0.015 μg/mL, 0.02 μg/mL, 0.025 μg/mL, 0.030 μg/mL, 0.035 μg/mL, 0.040 μg/mL, 0.045 μg/mL, 0.050 g/mL, 0.055 μg/mL, 0.060 μg/mL, 0.065 μg/mL, 0.070 μg/mL, 0.075 μg/mL, 0.080 μg/mL, 0.085 μg/mL, 0.090 μg/mL, 0.095 μg/mL, 0.10 g/mL, 0.15 μg/mL, 0.2 μg/mL, 0.25 μg/mL, 0.3 μg/mL, 0.35 μg/mL, 0.4 μg/mL, 0.45 μg/mL, 0.5 μg/mL, 0.55 μg/mL, 0.60 μg/mL, 0.65 μg/mL, 0.70 μg/mL, 0.75 μg/mL, 0.8 μg/mL, 0.85 μg/mL, 0.9 μg/mL, 1 μg/mL, 2 μg/mL, 3 μg/mL, 4 μg/mL, 5 g/mL, 6 μg/mL, 7 μg/mL, 8 μg/mL, 9 μg/mL, 10 μg/mL, 15 μg/mL, 20 μg/mL, 25 μg/mL, 30 g/mL, 35 μg/mL, 40 μg/mL, 45 μg/mL, 50 μg/mL, 55 μg/mL, 60 μg/mL, 65 μg/mL, 70 g/mL, 75 μg/mL, 80 μg/mL, 85 μg/mL, 90 μg/mL, 95 μg/mL, 100 μg/mL, 110 g/mL, 120 μg/mL, 130 μg/mL, 140 μg/mL, 150 μg/mL, 160 μg/mL, 170 μg/mL, 180 μg/mL, 190 μg/mL, 200 μg/mL, 250 μg/mL, 300 μg/mL, 350 μg/mL, 400 μg/mL, 450 μg/mL, 500 μg/mL, 550 μg/mL, 600 μg/mL, 650 μg/mL, 700 μg/mL, 750 μg/mL, 800 μg/mL, 850 μg/mL, 900 μg/mL, 950 μg/mL or more. In some embodiments, the upper boundary may be at least about 1000 μg/mL, 950 μg/mL, 900 μg/mL, 850 μg/mL, 800 μg/mL, 750 μg/mL, 700 μg/mL, 650 μg/mL, 600 μg/mL, 550 μg/mL, 500 μg/mL, 450 μg/mL, 400 μg/mL, 350 μg/mL, 300 μg/mL, 250 μg/mL, 200 μg/mL, 150 μg/mL, 100 μg/mL, 90 μg/mL, 95 μg/mL, 85 μg/mL, 80 μg/mL, 75 μg/mL, 70 μg/mL, 65 μg/mL, 60 μg/mL, 55 μg/mL, 50 μg/mL, 45 μg/mL, 40 μg/mL, 35 μg/mL, 30 μg/mL, 25 μg/mL, 20 μg/mL, 15 μg/mL, 10 μg/mL, 9 μg/mL, 8 μg/mL, 7 μg/mL, 6 μg/mL, 5 μg/mL, 4 μg/mL, 3 μg/mL, 2 μg/mL, 1 μg/mL, 0.9 μg/mL, 0.8 μg/mL, 0.7 μg/mL, 0.6 μg/mL, 0.5 μg/mL, 0.4 μg/mL, 0.3 μg/mL, 0.2 μg/mL, 0.1 μg/mL, 0.09 μg/mL, 0.08 μg/mL, 0.07 μg/mL, 0.06 μg/mL, 0.05 μg/mL, 0.04 μg/mL, 0.03 μg/mL, 0.02 μg/mL, 0.01 μg/mL, 0.009 μg/mL, 0.008 μg/mL, 0.007 μg/mL, 0.006 μg/mL, 0.005 μg/mL, 0.004 μg/mL, 0.003 μg/mL, 0.002 μg/mL or less. In some embodiments a “normal” level of sC5b-9 falls within the range of 0.001-1000 μg/mL; in other embodiments a “normal” level of sC5b-9 falls within the range of 0.001-750 μg/mL; in other embodiments a “normal” level of sC5b-9 falls within the range of 0.001-500 μg/mL; in other embodiments a “normal” level of sC5b-9 falls within the range of 0.01 μg/mL-500 μg/mL; in other embodiments a “normal” level of sC5b-9 falls within the range of 0.01 μg/mL-250 μg/mL; in other embodiments a “normal” level of sC5b-9 falls within the range of 0.05 μg/mL-250 μg/mL; in other embodiments a “normal” level of sC5b-9 falls within the range of 0.1 μg/mL-100 μg/mL; in other embodiments a “normal” level of sC5b-9 falls within the range of 0.5 μg/mL-50 μg/mL; in other embodiments a “normal” level of sC5b-9 falls within the range of 1-50 μg/mL.

In some embodiments, sC5b-9 is detected using a non-cross-reactive binding agent (e.g., antibody) characterized in that does not cross-react with C5 or C9.

### IL-6

Interleukin 6 (IL-6) can act as a pro-inflammatory cytokine or an anti-inflammatory myokine, depending on context and which will be understood by those of skill in the art. IL-6 can be secreted in response to pathogen-associated molecular patterns (PAMPS). PAMPS bind to pattern recognition receptors (PRRs) and toll-like receptors (TLRs), which are important components of the innate immune system. As will also be known to those of skill in the art, IL-6 can function in acute phases of inflammatory responses and may also be a marker of many chronic immune-mediated diseases, disorders and conditions.

In some embodiments, IL-6 may be measured in a sample in a range of 2-5000 pg/mL.

In some embodiments, a “normal” IL-6 level in a sample (e.g., bodily fluid) falls within a range with a lower boundary and an upper boundary that is higher than the lower boundary. In some embodiments, a “normal” IL-6 level in a sample falls within a range of 2-5000 pg/mL. In some embodiments, the lower boundary may be at least about 2 pg/mL, 3 pg/mL, 4 pg/mL, 5 pg/mL, 6 pg/mL, 7 pg/mL, 8 pg/mL, 9 pg/mL, 10 pg/mL, 15 pg/mL, 20 pg/mL, 25 pg/mL, 30 pg/mL, 35 pg/mL, 40 pg/mL, 45 pg/mL, 50 pg/mL, 55 pg/mL, 60 pg/mL, 65 pg/mL, 70 pg/mL, 75 pg/mL, 80 pg/mL, 85 pg/mL, 90 pg/mL, 95 pg/mL, 100 pg/mL, 125 pg/mL, 150 pg/mL, 175 pg/mL, 200 pg/mL, 225 pg/mL, 250 pg/mL, 275 pg/mL, 300 pg/mL, 325 pg/mL, 350 pg/mL, 375 pg/mL, 400 pg/mL, 425 pg/mL, 450 pg/mL, 475 pg/mL, 500 pg/mL, 525 pg/mL, 550 pg/mL, 575 pg/mL, 600 pg/mL, 625 pg/mL, 650 pg/mL, 675 pg/mL, 700 pg/mL, 725 pg/mL, 750 pg/mL, 775 pg/mL, 800 pg/mL, 825 pg/mL, 850 pg/mL, 875 pg/mL, 900 pg/mL, 925 pg/mL, 950 pg/mL, 975 pg/mL, 1000 pg/mL, 1100 pg/mL, 1200 pg/mL, 1300 pg/mL, 1400 pg/mL, 1500 pg/mL, 1600 pg/mL, 1700 pg/mL, 1800 pg/mL, 1900 pg/mL, 2000 pg/mL, 2100 pg/mL, 2200 pg/mL, 2300 pg/mL, 2400 pg/mL, 2500 pg/mL, 2600 pg/mL, 2700 pg/mL, 2800 pg/mL, 2900 pg/mL, 3000 pg/mL, 3100 pg/mL, 3200 pg/mL, 3300 pg/mL, 3400 pg/mL, 3500 pg/mL, 3600 pg/mL, 3700 pg/mL, 3800 pg/mL, 3900 pg/mL, 4000 pg/mL, 4100 pg/mL, 4200 pg/mL, 4300 pg/mL, 4400 pg/mL, 4500 pg/mL, 4600 pg/mL, 4700 pg/mL, 4800 pg/mL, 4900 pg/mL or more. In some embodiments, the upper boundary may be at least about 5000 pg/mL, 4900 pg/mL, 4800 pg/mL, 4700 pg/mL, 4600 pg/mL, 4500 pg/mL, 4400 pg/mL, 4300 pg/mL, 4200 pg/mL, 4100 pg/mL, 4000 pg/mL, 3900 pg/mL, 3800 pg/mL, 3700 pg/mL, 3600 pg/mL, 3500 pg/mL, 3400 pg/mL, 3300 pg/mL, 3200 pg/mL, 3100 pg/mL, 3000 pg/mL, 2900 pg/mL, 2800 pg/mL, 2700 pg/mL, 2600 pg/mL, 2500 pg/mL, 2400 pg/mL, 2300 pg/mL, 2200 pg/mL, 2100 pg/mL, 2000 pg/mL, 1900 pg/mL, 1800 pg/mL, 1700 pg/mL, 1600 pg/mL, 1500 pg/mL, 1400 pg/mL, 1300 pg/mL, 1200 pg/mL, 1100 pg/mL, 1000 pg/mL, 950 pg/mL, 900 pg/mL, 850 pg/mL, 800 pg/mL, 750 pg/mL, 700 pg/mL, 650 pg/mL, 600 pg/mL, 550 pg/mL, 500 pg/mL, 450 pg/mL, 400 pg/mL, 350 pg/mL, 300 pg/mL, 250 pg/mL, 200 pg/mL, 150 pg/mL, 100 pg/mL, 90 pg/mL, 80 pg/mL, 70 pg/mL, 60 pg/mL, 50 pg/mL, 40 pg/mL, 30 pg/mL, 20 pg/mL, 10 pg/mL, 9 pg/mL, 8 pg/mL, 7 pg/mL, 6 pg/mL, 5 pg/mL, 4 pg/mL, 3 pg/mL or less.

In some embodiments a “normal” level of IL-6 falls within the range of 2-5000 pg/mL; in other embodiments a “normal” level of IL-6 falls within the range of 2-4500 pg/mL; in other embodiments a “normal” level of IL-6 falls within the range of 2-4000 pg/mL; in other embodiments a “normal” level of IL-6 falls within the range of 2 pg/mL-3500 pg/mL; in other embodiments a “normal” level of IL-6 falls within the range of 2 pg/mL-3000 pg/mL; in other embodiments a “normal” level of IL-6 falls within the range of 2 pg/mL-2500 pg/mL; in other embodiments a “normal” level of IL-6 falls within the range of 2 pg/mL-1500 pg/mL; in other embodiments a “normal” level of IL-6 falls within the range of 5 pg/mL-1000 pg/mL; in other embodiments a “normal” level of IL-6 falls within the range of 5-500 pg/mL; in other embodiments, a “normal” level of IL-6 falls within the range of 5-250 pg/mL; in other embodiments, a “normal” level of IL-6 falls within the range of 5-125 pg/mL; in other embodiments, a “normal” level of IL-6 falls within the range of 10-100 pg/mL; in other embodiments, a “normal” level of IL-6 falls within the range of 25-75 pg/mL.

In some embodiments, IL-6 is detected using a non-cross reactive binding agent (e.g., antibody).

### ADAMTS13

A disintegrin and metalloproteinase with thrombospondin-1 motif 13th family member (ADAMTS13) is an enzyme that cleaves von Willebrand factor (VWF), which is a protein involved in blood clotting. Inherited or acquired deficiencies in plasma ADAMTS13 are associated with one or more diseases, disorders or conditions. In some embodiments, ADAMTS13 deficiencies cause TTP. Timely and accurate diagnosis of TTP is very important. Because TTP can have similar symptoms to other diseases such as aHUS, each of which has different treatments. Accordingly, it is important to have an assay that can quickly, sensitively, and specifically differentiate between TTP and another diseases such as aHUS, including because treatment of a subject in need thereof may vary greatly depending upon diagnosis.

ADAMTS13 levels can be measured as a percentage of activity by combining a sample and a substrate comprising a peptide and then assayed for percentage of cleaved peptide. Previously available assays are burdened by extensive steps such as many serial dilutions and long time periods to completion (e.g., as in standard ELISA assays). For example, even the fastest previously available assays take a minimum of 1-3 hours. In addition, only a single target (e.g., ADAMTS13) can be measured in those assays. In contrast, the present disclosure provides technologies that markedly improve (e.g., reduce) the amount of time needed to achieve results. That is, in some embodiments, the present disclosure provides methods and assays that provide results within 35 minutes of obtaining a sample. As will be appreciated by those of skill in the art, this is an important improvement and makes this crucial assay very amenable to point-of-care formats. Furthermore, in some embodiments, after a sample is contacted with a substrate, one or more targets in that sample/substrate mixture can be measured using an immunoassay device of the present disclosure. In some such embodiments, the present disclosure provides technologies that not only allow measurement of percent activity of ADAMTS13, but are multiplexed with one or more other targets, which are measured on the same immunoassay device at the same time as ADAMTS13 is measured. The decreased time to result and multiplexing of ADAMTS13 measurements with one or more other targets represent vast improvements in functional utility of this assay as compared to previously available assays.

In some embodiments, ADAMTS13 may be measured in a sample in a range of <10-100% activity (as measured by amount of substrate cleavage).

In some embodiments, a level of ADAMTS13 activity is 10% or less. In some embodiments, a level of ADAMTS13 activity within a range where a lower boundary is at least about 10, 15, 20, 25, 30, 35, 40, 45, 50, 55, 60, 65, 70, 75, 80, 85, 90, 95% or greater. In some embodiments, a level of ADAMTS13 activity is within a range where an upper boundary is at least about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11% or less.

In some embodiments, ADAMTS13 is detected using a recombinant VWF substrate.

In some embodiments, a non-cross-reactive antibody that recognizes only a cleaved VWF fragment will be used for detection.

### Ba

Ba is a cleavage product of the alternative complement pathway. In some instances, cleavage of the thioester bond in C3 by a water molecule form C3(H₂O), which binds factor B and then allows factor D to cleave factor B to Ba and Bb. Bb remains associated with C3(H₂O) to form C3(H₂O)Bb complex, which acts as a C3 convertase and cleaves C3, resulting in C3a and C3b. Ba has been found to reduce lymphocyte activity (e.g., proliferation).

In some embodiments, Ba may be measured in a sample in a range of 0.5-20 μg/mL.

In some embodiments, a “normal” Ba level in a sample (e.g., bodily fluid) falls within a range with a lower boundary and an upper boundary that is higher than the lower boundary. In some embodiments, a “normal” Ba level in a sample falls within a range of 0.5-20 μg/mL. In some embodiments, the lower boundary may be at least about 0.5 μg/mL, 0.6 μg/mL, 0.7 μg/mL, 0.8 μg/mL, 0.9 μg/mL, 1 μg/mL, 2 μg/mL, 3 μg/mL, 4 μg/mL, 5 μg/mL, 6 μg/mL, 7 μg/mL, 8 μg/mL, 9 μg/mL, 10 μg/mL, 11 μg/mL, 12 μg/mL, 13 μg/mL, 14 μg/mL, 15 μg/mL, 16 μg/mL, 17 μg/mL, 18 μg/mL, 19 μg/mL or more. In some embodiments, the upper boundary may be at least about 20 μg/mL, 19 μg/mL, 18 μg/mL, 17 μg/mL, 16 μg/mL, 15 μg/mL, 14 μg/mL, 13 μg/mL, 12 μg/mL, 11 μg/mL, 10 μg/mL, 9 μg/mL, 8 μg/mL, 7 μg/mL, 6 g/mL, 5 μg/mL, 4.5 μg/mL, 4 μg/mL, 3.5 μg/mL, 3 μg/mL, 2.5 μg/mL, 2 μg/mL, 1.9 μg/mL, 1.8 μg/mL, 1.7 μg/mL, 1.6 μg/mL, 1.5 μg/mL, 1.4 μg/mL, 1.3 μg/mL, 1.2 μg/mL, 1.1 μg/mL, 1.0 μg/mL, 0.9 μg/mL, 0.8 μg/mL, 0.7 μg/mL, 0.6 μg/mL, or less. In some embodiments a “normal” level of Ba falls within the range of 0.5 μg/mL-20 μg/mL; in other embodiments a “normal” level of Ba falls within the range of 0.5 g/mL-15 μg/mL; in other embodiments a “normal” level of Ba falls within the range of 1 μg/mL/mL-10 μg/mL; in other embodiments a “normal” level of Ba falls within the range of 5 μg/mL-7.5 μg/mL.

In some embodiments, Ba is detected using a non-cross reactive binding agent that does not cross-react with B or Bb.

### Other Immune Targets

In some embodiments, one or more additional targets will be measured. By way of non-limiting example, in some embodiments, one or more targets may include a MASP2:AT complex. The superfamily of serine protease inhibitors (serpins), namely C1 inhibitor (C1-INH) and antithrombin (AT), regulate MASP-2 activity by forming stable complexes with activated MASP-2. Thus, MASP-2 activation can be assessed by measurement of complexes between MASP-2 and its regulating serpins. MASP-2 activation is preferentially regulated by C1-INH when triggered by lectin pathway pattern recognition molecules. However, AT is the primary regulator of MASP-2 activation in the presence of clotting blood. MASP/serpin complexes in SLE are correlated with platelet activation parameters. Analogous to MASPs, the contact protease FXII is activated by fibrin clots and regulated by AT and C1-INH. Increased FXIIa/AT and decreased FXIIa/C1-INH have been shown to greatly increase the odds for vascular disease in SLE. AT complex formation is highly correlated with thrombotic reactions. Complement is activated by non-biological surfaces, such as a ventilator. It has been shown that C4/C4BP and FXII/C1-INH absorbed to non-biological surfaces correlates with pro-inflammatory cytokine generation. Conversely, the complement activation fragments C3a, C5a and C5b-9 have poor predictive value as biocompatibility markers.

In some embodiments, one or more additional targets measured will include C4d, Ba, Bb, FH, CXCL9, sCD25, microRNA, IL8, Pentraxin3, IL1, VCAM1, thrombomodulin, ferritin, CRP, IL-10, TNFα, IFNγ, and/or creatinine. As will be known to those of skill in the art, one or more diseases, disorders, or conditions or risk thereof may be characterized by presence, absence, increased, and/or decrease in measurements of one or more targets. For example, in some embodiments, one or more targets listed in Table 1 may be combined into one or more panels of targets to be measured from a sample. Regardless of whether one or more targets is known to be a target of interest in a particular disease, disorder, or condition (e.g., in assessing risk or diagnosis), the present disclosure provides technologies that not only measures one or more targets, but can do so with increased efficiency, accuracy, specificity, sensitivity, reliability, and/or speed as compared to previously available assays. Importantly, technologies of the present disclosure also improve latency between obtaining a sample for measurements and receipt of measurement.

TABLE 1
Exemplary Targets and Ranges

| Target | Measurement Range [LLOQ-ULOQ] | |
|---|---|---|
| Intact C3 | 0.025-5 | mg/mL |
| C3a | 1-3000 | ng/mL |
| iC3b | 0.2-50 | μg/mL |
| Intact C4 | 0.05-5 | mg/mL |
| C5 | 0.001-1000 | μg/mL |
| sC5b-9 | 0.001-1000 | μg/mL |
| IL-6 | 2-5000 | pg/mL |
| ADAMTS13 | <10%-100% | cleavage |
| Ba | Blood/plasma: 0.5-20 μg/mL | |
| | Urine: 1-5000 ng/mL | |
| microRNA | Positive or negative; | |
| | detection limit currently as low 1 μM miRNA | |
| CRP | 0.1-100 | μg/mL |
| Creatinine | 6.2-8000 | μg/mL |
| C4d | 0.5-50 | μg/mL |

In some embodiments, one or more targets from Table 1 may be measured from one or more samples from a single patient. In some embodiments, the one or more targets may be measured on the same immunoassay device. In some embodiments, one or more targets may be measured on the same or different test strips within a given immunoassay device. In some embodiments, one or more targets may be measured on different immunoassay devices, dependent on context and targets.

### Panels

In some embodiments, one or more combinations of targets may be combined to generate a panel. In some such embodiments, such a panel will be reflected in technologies as provided herein. For example, in some embodiments, a panel will comprise targets that can be measured using a single cassette or multiple cassettes, each cassette comprising an immunoassay device as provided herein. For example, in some embodiments, targets in a panel will all be on a single cassette (i.e., all targets would be measured on test strips within a single test cassette. As will be understood by those of skill in the art, targets may be combined and measured using a single immunoassay device with capture agents provided on one or more test strips and test lines within limits of chemical compatibility of all components of a particular immunoassay device and its components (e.g., capture and detecting agents, etc.).

In some embodiments, a panel may comprise one or more of C3, C3a, iC3b, C4, C5, sC5b-9, IL-6, ADAMTS13, MASP2:AT complex, C4d, Ba, Bb, FH, CXCL9, sCD25, microRNA, IL8, Pentraxin3, IL1, VCAM1, thrombomodulin, ferritin, CRP, IL-10, TNFα, IFNγ, and/or creatinine.

In some embodiments, a panel may be or comprise Ba and sC5b-9, with or without creatinine. In some embodiments, a panel may comprise C3 and iC3b. In some embodiments, a panel may comprise C3 and C3a. In some embodiments, a panel may comprise sC5b-9 and ADAMTS13. In some embodiments, a panel may be or comprise sC5b-9 with or without creatinine. In some embodiments, a panel may be or comprise IL6 with or without CRP. In some embodiments, a panel may be or comprise C3, iC3b and/or IL6 and, optionally, CRP. In some embodiments, a panel may be or comprise iC3b, C3, and C4. In some embodiments, a panel may be or comprise C3, C3a and, optionally, iC3b, sC5b-9, C5, C4, C4d, IL-6, and/or IL-1. In some embodiments, a panel may be or comprise C3, C3a, iC3b, sC5b-9, C5, C4, C4d, IL-6, and/or IL-1. In some embodiments, a panel may be or comprise iC3b, Ba, sC5b-9 and, optionally, creatinine or CRP. In some embodiments, a panel may be or comprise sC5b-9, ADAMTS13 activity, iC3b, C3, C4, C4d, Ba, and/or free C5; in some such embodiments, C5 may be measured in a sample comprising an anti-C5 therapeutic. In some embodiments, a panel may be or comprise sC5b-9, ADAMTS13, C3, C4, C4d, Ba, IL-8 and, optionally, IL-6, iC3b, and/or free C5. In some embodiments, a panel may be or comprise iC3b, sC5b-9, intact C3 and/or IL-6 and creatinine if a sample is a urine sample or iC3b, sC5b-9, C4, C4d, intact C3, and/or IL-6, and, optionally, free C5 (i.e., if a patient is on an anti-C5 therapeutic). In some embodiments, a panel may be or comprise IL-6, IL-1, C5a, iC3b, CXCL9, sC5b-9, sCD25, Ferritin, and/or MASP2:AT. In some embodiments, a panel may be or comprise IL-6, IL-1, C5a, iC3b, CXCL9, sCD25, Ferritin, sC5b-9, and/or MASP2:AT.

### Samples

Any of a variety of samples may be analyzed using technologies of the present disclosure. For example, one or more targets may be measured in one or more samples as described herein. In some embodiments, a sample is a biological sample. In some embodiments, such a biological sample is obtained or derived from a source of interest as described herein. In some such embodiments, such a source may be a commercially available source. In some embodiments, a source of interest comprises an organism, such as an animal or human. In some embodiments, a sample comprises one or more targets. In some such embodiments, samples comprising such targets are measured using technologies of the present disclosure. In some embodiments, measurement of a sample reveals that the sample does not comprise one or more targets. That is, in some embodiments, a level of a target may be zero or below a LLOQ for that target.

In some embodiments, a biological sample is a liquid sample. In some embodiments, a biological sample is or comprises biological tissue or fluid. In some embodiments, a sample is or comprises a liquid. In some such embodiments, a liquid is or comprises one or more body fluids, or is derived therefrom. In some embodiments, a sample is or comprises a body fluid selected from the group consisting of whole blood, serum, plasma, urine, tears, saliva, feces, wound exudate, pus, nasal discharge, bronchoalveolar lavage fluid, mucous secretion, sebum, sweat, semen, vaginal fluid, breast milk, breath condensate, and cerebrospinal fluid.

In some embodiments, a measurement of a given target may differ by one or more orders of magnitude depending upon the particular sample. For instance, by way of non-limiting example, in some embodiments, a “normal” level of C3 in blood may be between about 0.05-5 mg/mL, whereas a “normal” level of C3 in cerebrospinal fluid may be between about 0.025-0.1 mg/mL. In some embodiments, a “normal” level of Ba in plasma may be between about 0.7-1.3 μg/mL, whereas in urine, a “normal” level of Ba may be about 1-29 ng/ml. As will be understood by those in the art, given context, a level of a particular target in one sample comprising a first fluid may be at least one, two, three, four, five, six, seven, eight, nine, ten or more orders of magnitude higher or lower than in a second sample comprising a second fluid, wherein the second fluid is not the same as the first fluid.

In some embodiments a sample is or comprises a solution such as, e.g., a buffer, to which a target has been added. For example, in some such embodiments, a sample is or comprises a buffer to which a target has been added for purposes of testing one or more assays or developing one or more reference ranges or control conditions for a target. In some embodiments, a sample is not solid or substantially comprised of solid material. In some embodiments, a liquid sample is or comprises cells or tissue collected and suspended, mixed or otherwise contacted with a liquid (e.g., a swab placed in a buffer). In some embodiments, a liquid or fluid sample or portion thereof is concentrated after collection (e.g., a urine sample).

In some embodiments, a biological sample may be or comprise bone marrow; blood (e.g., whole blood); blood cells; ascites; tissue or fine needle biopsy samples; cell-containing body fluids; free floating nucleic acids; serum; plasma; sebaceous secretions (e.g., sebum, e.g., smegma), semen; milk; breath condensate; sputum; saliva; urine; cerebrospinal fluid; ocular fluid (e.g., vitreal fluid); peritoneal fluid; pleural fluid; feces; lymph; gynecological fluids (e.g., vaginal fluid); wound exudate; pus; skin swabs; vaginal swabs; oral swabs; nasal swabs; washings or lavages such as a ductal lavages or broncheoalveolar lavages; aspirates; scrapings; bone marrow specimens; synovial fluid; tissue biopsy specimens; surgical specimens; feces, other body fluids, secretions, and/or excretions such as sweat and tears; and/or cells therefrom, etc. In some embodiments, a biological sample is or comprises cells obtained from an individual. In some embodiments, obtained cells are or include cells from an individual from whom the sample is obtained.

In some embodiments, a sample is a “primary sample” obtained directly from a source of interest by any appropriate means. For example, in some embodiments, a primary biological sample is obtained by methods comprising a biopsy (e.g., fine needle aspiration), surgery, and/or collection of body fluid (e.g., blood, lymph, urine etc.), etc. In some embodiments, a primary sample is a crude sample. In some such embodiments, a crude sample is substantially unprocessed. For example, in some embodiments, a sample may be collected, and an aliquot of the sample may be taken directly from the collection vehicle and applied to a test strip of the present disclosure (e.g., urine, e.g., whole blood, e.g., tears, e.g., cerebrospinal fluid, etc.). In some embodiments, as will be clear from context, the term “sample” refers to a preparation that is obtained by processing (e.g., pre-processing, e.g., by removing one or more components of and/or by adding one or more agents to) a primary sample, i.e., a “processed sample.” For example, in some embodiments, a processed sample may be a sample filtered using a semi-permeable membrane.

In some embodiments, a sample requires different conditions for measurement of a given target than for measuring the same target from a different type of sample. For instance, in some embodiments, measuring a target in blood and measuring the same target in urine may require different conditions. By way of non-limiting example, among other things, the present disclosure provides insight that measuring a target in a sample comprising urine may, in some embodiments, require different conditions and normalization procedures as compared to measurement of the same target in a sample comprising blood. As described herein, disclosed technologies provide a solution to this challenge and, when performed under appropriate conditions, a target may be measured in any liquid sample, given appropriate context.

Without being bound by any particular theory, the present disclosure contemplates that in contrast to previously available assays, in some embodiments, one or more targets can be measured in urine with improved efficiency, sensitivity, accuracy, specificity, reliability, and/or speed. For instance, in some embodiments, sC5b-9 is an exemplary target that requires different conditions when measured in a sample comprising blood and a sample comprising urine. That is, in some embodiments, sC5b-9 may be a low abundance target in urine (but not, e.g., in blood). Accordingly, due to the low abundance nature of this target, previously available sC5b-9 assays were not capable of accurately measuring (or even detecting) sC5b-9 in urine. Thus, previously available assays often erroneously reported absence of this target due to insufficient LLOQ.

As described herein, the present disclosure recognizes that, in some embodiments, factors such as pH of a sample must be adjusted to account for sample composition. In some such embodiments, buffering and pH ranges will be dependent upon context and sample composition (e.g., blood, plasma, urine, tears, etc.) and will be understood by those of skill in the art. For example, in some embodiments, pH of a sample comprising urine may require different buffering conditions and pH ranges that those for use with a sample comprising blood or plasma. Accordingly, in some such embodiments, a sample pad of the present disclosure comprises a buffer appropriate for the sample being measured on a test strip. For example, in some embodiments, a sample pad comprises a buffer that is appropriate for a urine-compatible pH range. In some embodiments, certain samples may require additional filtering or separation steps prior to measurement on a test strip. For example, in some embodiments, measurements of samples comprising whole blood require removal of red blood cells prior to contacting the test strip. In some such embodiments, a filter pad comprises a method of removing red blood cells (e.g., an anti-RBC antibody) from the sample prior to the sample contacting and traveling into and through a test strip.

The present disclosure also recognizes that volume of a sample may impact measurements of one or more targets. For example, in some embodiments, variable urine concentration may drastically impact measurements of sC5b-9. Accordingly, in some embodiments, technologies of the present disclosure provide means for measuring a target used to normalize a sample. By way of non-limiting disclosure, in some embodiments, when sC5b-9 is measured from a sample comprising urine, creatinine is also measured. As will be appreciated by those of skill in the art, this advance in capability to multiplex and efficiently, accurately, sensitively, specifically, reliably and/or rapidly measure at least two targets allows for normalization of the sample, accounting for differences in, e.g., volume and concentration. For example, in the case of measuring sC5b-9 in urine, ability to multiplex and measure both sC5b-9 and creatinine allows accurate measurements of sC5b-9 to be measured because creatinine can be used to normalize the results obtained from the sC5b-9 assay.

The present disclosure provides the insight that, in some embodiments, intentionally diluting a sample such as is performed in a standard ELISA, may result in inaccurate measurements. That is, in some embodiments, dilution of a sample (e.g., substantially diluting a sample, diluting a sample at all, e.g., diluting prior to application of the sample to a test strip, immunoassay device, and/or test cartridge as described herein) may be problematic for accurately measuring a target. For instance, the present disclosure recognizes that, in some embodiments, dilution may dissociate a target from a therapeutic and, thus, provide an erroneous measurement of an amount of target in a sample, due to post-collection events. By way of non-limiting example, in some embodiments, one or more targets may be measured in one or more samples from a subject with the intent of monitoring response to an administered therapeutic. For instance, in some embodiments, an individual may be administered a therapeutic (e.g., an anti-C5 antibody, an anti-C3 antibody) that binds to a particular target. In some such embodiments, measurement of the unbound or uncomplexed “free” target may be used to determine whether a change in treatment, such as increase or decrease in dosage of the therapeutic, is needed. As described herein, previously available assays generally require several serial dilutions in order to accurately measure a target and some therapeutic/target complexes are particularly vulnerable to dissociation upon dilution. This vulnerability causes previously available assays to inaccurately report level of a target due to dissociation of the target from the complex and, in some embodiments, may cause a provider to change a treatment when no change is actually needed.

Furthermore, in some embodiments, certain samples may be vulnerable to post-collection, pre-measurement changes due to sensitivity of one or more targets being measured. For example, in some embodiments, measurements of iC3b may be falsely elevated if a sample is extensively handled prior to measurement. In some embodiments, diluting a sample may increase amount of time needed to process a sample and, thus, increase amount of time to results and treatment of a subject in need thereof. In some embodiments, diluting a sample may introduce additional sources of error. In some embodiments, diluting a sample may require additional resources (e.g., equipment, personnel, etc.) which may impact resources for performing measurements and treating subjects in need thereof. The present disclosure provides technologies that use samples that have not been subject to such extensive pre-processing such as several serial dilutions or repeated pipetting and, instead, are maintained as substantially neat (e.g., undiluted, unseparated, minimally diluted, no offline dilutions, separations, or purification events, etc.). In some such embodiments, substantially neat samples are measured as provided herein without any further manipulation. For example, in some embodiments, substantially neat samples are placed in contact with a test cartridge of the present disclosure and measured within a certain period after collection and without significant intervening steps.

Thus, in some embodiments, the present disclosure provides technologies that improve methods of measuring one or more targets and/or monitoring one or more therapeutic agents by overcoming challenges related to measurements including dilution-driven dissociation and latency and/or handling between sample collection and measurement.

In some embodiments, a sample is obtained and applied to a test cartridge in less than 60 minutes (e.g., less than 50, 40, 30, 20, 10 minutes) from time of obtaining the sample. Upon contact with a test cartridge, the sample interacts with one or more components such as a sample pad, conjugate pad, test strip, test line, capture agent, competing agent, or detecting agent within approximately six to ten seconds and takes approximately 60-90 seconds to fully move across the length of a test strip. Accordingly, in some such embodiments, technologies of the present disclosure solve problems of previously available assays by eliminating any requirement for significant sample dilution in order to measure a target in a sample.

In some embodiments, a sample is obtained and processed immediately. In some embodiments, a sample is obtained and stored prior to measuring. In some embodiments, a sample is collected and split into two or more portions. In some such embodiments, at least one portion of the sample is processed immediately (e.g., within 30 minutes or less of obtaining). In some embodiments, at least a portion of the sample is pre-processed (e.g., contacted with an agent for at least 5 minutes) and then measured immediately (e.g., within 30 minutes). In some such embodiments, at least one portion of a sample is reserved (e.g., stored for later analysis, banking, etc.). In some embodiments, a sample is processed within 60 minutes or less of being obtained. In some embodiments, a sample is processed within 30 minutes or less of being obtained. In some embodiments, a sample is kept cool (e.g., kept on ice) for up to 5 hours post-obtaining (e.g., collection or thawing). In some embodiments, a sample may be divided with a portion of a sample being measured immediately and a portion reserved and stored (e.g., frozen, etc.).

In some embodiments, a sample is applied to an immunoassay device (e.g., via a sample port) of a test cassette. In some embodiments, a sample is applied to an immunoassay device as quickly as possible after a sample is obtained. In some embodiments, a sample is applied within 30 minutes, but no more than five hours, of being obtained. In some embodiments, if a sample is applied to an immunoassay device greater than approximately 30 minutes from being obtained, the sample is held under cooled conditions (e.g., on ice). In some embodiments, amount of time a sample is left at ambient temperature is minimized as much as possible (e.g., to ensure reproducibility and accuracy of measurements). In some embodiments, if testing is not available for freshly obtained samples, samples may be measured after storage if sample handling is optimized prior to and during storage. For example, such optimization may include, but not be limited to, minimal time at room temperature, quick processing of sample (e.g., into plasma when a sample comprises whole blood), and transfer to storage (−80 C) until ready to assess on an assay as disclosed herein. In some embodiments, a previously frozen sample should be measured immediately upon thawing.

In some embodiments, a sample may comprise a concentration of a target that is in such low abundance that a lower limit of quantification is still too great (i.e., false negative results/inaccurate measurements result). Accordingly, in some embodiments, in order to provide an efficient, accurate, sensitive, specific, reliable, and/or rapid measurement and do so with a sample comprising a low abundance target benefits from assays that do not require dilution or washing steps that can result in loss of material (e.g., sample material, e.g., target). In some such embodiments, an assay may require additional correction factors. For example, in some embodiments, a target is sC5b-9 and a sample is urine. In some embodiments, sC5b-9 is a low abundance target in urine. Accordingly, in some such embodiments, an assay measuring sC5b-9 also measures creatinine and is further optimized (e.g., buffer composition, e.g., pH) to accurately measure such targets in urine, unlike previously available assays which are not optimized or normalized for sample type. In some embodiments, such assay improvements avoid inaccurate measurements including, but not limited to false negatives of a given target.

The present disclosure recognizes that measurement of samples comprising one or more high abundance targets may be problematic due to limits of quantitation ranges. For instance, measuring certain targets (e.g., C5, C3) using previously available assays may not be able to produce results that distinguish between measurements beyond a certain level. Thus, in some embodiments, a sample may comprise an amount of a target that is present in such high abundance that an upper limit of quantification (ULOQ) of an assay is exceeded. For example, a high abundance target may saturate a detecting agent so that accurate measurements are not possible within a range measured by an assay. In addition, a complicating factor to samples comprising at least one high abundance target is that previously available assays addressed concentration challenges using several serial dilutions. As described herein, diluting certain samples, including those that may commonly have high abundance targets such as C5 or C3, can also dissociate a target from a therapeutic in a sample that comprises complexes of target and therapeutic (e.g., anti-C5, anti-C3, etc.).

Accordingly, the present disclosure provides solutions to this problem. Importantly, these solutions do not include use of offline dilutions, which risk dissociation of a target as described herein. Thus, in some embodiments, technologies of the present disclosure provide assays and devices that use one or more test lines to functionally dilute a sample as it travels along a test strip. That is, in some embodiments, a test line comprises an agent such as a capture agent that captures an amount of a target to effectively perform an inline dilution and reduce a quantity of a target such that further test lines on the test strip can be used to quantify the target within detectable ranges. For instance, by way of non-limiting example, a test strip is contacted by a sample comprising at least one high abundance target. As the target enters and travels on the test strip, a first test line comprising a capture agent is contacted, and a portion of the target is retained by the capture agent on the test line while the remainder of the sample continues to travel through the strip towards one or more additional test lines. In some embodiments, a second test line may comprise the same capture agent to remove an additional portion of a target from a sample. In some such embodiments, such assays are performed, and a high abundance target is measured without a need to perform any steps (e.g., offline dilution) to bring an amount of a target in a sample within a range that an assay can measure.

The present disclosure also provides advantages for clinical trials. For example, since significant or several serial dilutions are not required nor is repetition of various replicates required as in previously available assays, results can remain “blinded” to investigators performing assays. That is, in some previously available assays, particularly in conditions with high abundance targets, an investigator might have encountered a situation where a sample was not dilute enough to fall within a range of an assay and needed to rerun the assay with further dilutions, effectively unblinding the test and sample(s) by indicating that a particular sample had a particularly high concentration of a specific target. Technologies of the present disclosure overcome this challenge.

### Pre-Processing

In some embodiments, a sample may be pre-processed. In some embodiments, pre-processing may be or comprise an offline dilution and/or an inline dilution; that is, in some embodiments, a sample may be combined with another agent (e.g., a substrate) prior to measuring one or more targets in the sample. Such a processed sample may comprise, for example, substantially undisturbed materials such as those that have not been processed to specifically remove or expose nucleic acids or purified materials such as protein or mRNA, other than whichever component(s) is/are removed with one or more processing steps.

As will be understood by one of skill in the art, in some embodiments, given context, pre-processing (e.g., by dilution) may be desirable or needed when a sample is or comprises a high concentration target. In some embodiments, pre-processing may be desirable or needed when a sample is or comprises a viscosity level that may be incompatible with any components of technologies provided herein. In some embodiments, pre-processing may be desirable or needed when measuring activity of a target such as, for example, ADAMTS13, which is measured in percent activity (not, e.g., measuring particular concentration or quantity). In some embodiments, a sample may be pre-processed to make it compatible with one or more steps of technologies (e.g., assays) as provided herein. In some embodiments, a sample may be pre-processed by performing one or more steps to “activate” a sample such as for assessing activity of a target such as a complement regulator. For example, in some embodiments, a sample may be pre-processed by an offline addition of a buffer or sample stabilizer to, for example, stabilize a sample or alter a pH of a sample. In some embodiments, such a step may occur inline (e.g., in a pad of prior to contact with a test strip). Activity could also apply to activating the sample and assessing for the activity of a complement regulator, among other utilities.

In some embodiments, an optional pre-processing step may be performed. That is, in some embodiments, a sample may be pre-processed by performing one or more steps that modify the sample in some way after obtaining the sample and prior to measurement of one or more targets. In some embodiments, a sample may be pre-processed when a measurement of one or more targets is expected to be so high that it will not fall into a measurable range in an assay. In some embodiments, a pre-processing step comprises contacting a sample with a liquid. In some embodiments, a liquid may be a buffer or other suitable agent for mixing with the sample. In some embodiments, mixing the sample with a liquid dilutes the sample. In some embodiments, the liquid comprises at least one agent and the liquid comprising, and agent is mixed with the sample. In some embodiments, the agent is a substrate. For example, in some embodiments, an agent is a substrate which will react with a particular target, if the target is present in the sample.

In some embodiments, a target is present in a sample and an agent is modified. In some embodiments, a target is not present or is not present in sufficient concentration to cause any modification to a substrate. In some embodiments, a sample is mixed with a liquid, which liquid optionally comprises an agent, and the mixture is allowed to sit (e.g., react) for a period of time. In some embodiments, the period of time is one, two, three, four, five, six, seven, eight, nine, or ten minutes. In some embodiments, after a sample is mixed with liquid comprising an agent and allowed to sit for a period of time, the sample (or a portion thereof) is applied to a test strip. For example, in some such embodiments, a sample is applied to a port in a test cassette and the sample contacts a sample and/or conjugate pad prior to contacting the test strip.

In some embodiments, a sample is pre-processed by separation. In some such embodiments, separation may be or comprise centrifugation of a sample. For instance, in some embodiments, a sample comprising whole blood may be obtained and processed to separate components (e.g., red blood cells, plasma, serum). In some such embodiments, several types of samples may be obtained and, optionally measured, reserved for later measurement, or discarded. As described herein, a sample is not considered pre-processed if it is modified after application to a test cassette, such as application to a sample pad comprising means to remove red blood cells, as described herein. That is, in some embodiments when something is removed from a sample after it contacts a component of an immunoassay device or test cassette, a sample is not considered pre-processed. For instance, contacting a sample to a sample pad comprising an anti-red blood cell antibody and/or filter prior to the sample entering a test strip is not considered pre-processing. In some embodiments, however, a step of pre-processing may be optionally followed by contacting a sample to a sample pad comprising an agent that also removes a portion of the sample (e.g., red blood cells using an anti-RBC antibody, etc.).

In some embodiments, a pre-processing step is or comprises a dilution step. In some such embodiments, a dilution is performed with one or more components. For example, in some embodiments, a dilution may be performed using a sample buffer. In some embodiments, a dilution step comprises addition of an agent. In some such embodiments, an agent is or comprises a substrate. For example, in some embodiments, a dilution is performed using a liquid that is or comprises a substrate that may be modified by one or more targets in a sample. For example, in some embodiments, a substrate is an enzyme, and a modification is cleavage. In some embodiments, a dilution may be performed using a combination of sample buffer and a substrate. As described herein, in some embodiments, a sample may be combined with a solution comprising an agent (e.g., a substrate) and the combination is allowed to sit for a period of time prior to contact with an immunoassay device or test strip therein. In some such embodiments, where a sample is “pre-processed,” the sample is then applied to an immunoassay device for measurement of one or more targets.

In some embodiments, pre-processing may occur offline. In some embodiments, pre-processing may occur inline. In some embodiments, where pre-processing is a dilution and the dilution is an offline dilution, a sample is diluted after it is obtained and prior to contacting a test strip (e.g., of an immunoassay device). As will be appreciated by those of ordinary skill in the art, when referring to previously available assays, dilution generally refers to an offline dilution in which a sample is collected and then a liquid is added to the sample at predetermined and measured intervals in order to generate one or more diluted samples (e.g., 1, 1:10, 1:20, 1:50, 1:100, 1:1000, etc.). In some such embodiments, one or more targets are measured in such diluted samples using a standard assay such as an ELISA or other similarly-arranged assay. As discussed throughout, one of the insights provided by the present disclosure is that such serial dilutions (i.e., as are common in certain previously available assays) can result in inaccurate target measurements due to complicating factors such as saturation from high abundance targets, failure to detect low abundance targets, or targets vulnerable to dilution-induced dissociation.

In some embodiments, after a sample is obtained or collected, pre-processing comprising one or more steps may be performed such that a sample is subject to a minimal dilution. In some embodiments, a minimal dilution does not comprise a serial dilution or several serial dilutions. A minimal dilution may quantitatively differ depending on a target, sample, and assay. For example, as will be clear to one of skill in the art, given context, a “minimal” dilution of a sample comprising C3 may be considered “minimally” diluted at 1:1000 as compared to 1:10000 in previously available assays or as compared to several serial dilutions of a sample.

In some embodiments, a dilution is performed during sample preparation. For example, in some embodiments, a preprocessing step includes combining a sample with another agent (e.g., a substrate, e.g., rVWF for an ADAMTS13 cleavage assay). In some such embodiments, a dilution is dependent upon the sample and the substrate. For example, in some embodiments, in an ADAMTS13 assay, a dilution is a 1:10 dilution of sample and rVWF substrate in solution.

In some embodiments, a dilution is performed using a sample buffer substantially similar to that used in a measurement performed using a test cassette or test cartridge.

In some embodiments, a dilution is an inline dilution. For example, in some embodiments, a dilution involves applying a sample to a test cassette as provided by the present disclosure. In some such embodiments, once the sample contacts the immunoassay device of the test cassette (i.e., contacts at least one test strip comprising at least one test line), a sample is considered diluted with respect to a concentration of at least one target after the target has been captured by, for example, a competing agent so that it is immobilized and prevented from being captured and/or detected by subsequent test lines.

### Assays

According to various embodiments the present disclosure provides technologies (e.g., methods, devices, etc.) for measuring one or more targets in one or more samples. In some embodiments, the present disclosure provides assays, devices, and methods of manufacturing, characterization, and uses thereof. In some embodiments, one or more assays and/or methods of use thereof are new or improved relative to previously available assays. In some embodiments, one or more components of one or more assays are new and/or improved relative to previously available assays and components.

In some embodiments, assays of the present disclosure comprise contacting an immunoassay device comprising at least one test strip with a sample. In some embodiments, the sample is applied to a test cassette (i.e., comprising an immunoassay device which itself comprises at least one test strip comprising one test line). In some such embodiments, when a sample is applied to a test cassette, the sample contacts the immunoassay device. In some such embodiments, the sample contacts a conjugate pad and/or a sample pad prior to contacting at least one test strip. In some embodiments, a sample is applied to a test cassette through a port.

In some embodiments, where a sample contacts a test cassette and contacts a sample and/or conjugate pad, the sample interacts with one or more reagents prior to contacting a test strip.

In some embodiments, an assay of the present disclosure requires one or more pre-processing steps prior to contacting a test cassette with a sample. For instance, in some embodiments, a sample may be contacted with an agent in a vessel or container such that the agent and sample are combined prior to contacting a test cassette. In some embodiments, the pre-processed sample or portion thereof is applied to the test cassette after a certain period of time. In some embodiments, a sample is collected or obtained, and the sample, or portion thereof, is combined with an agent (e.g., a substrate, e.g., a recombinant substrate). In some such embodiments, the sample contacted with the agent is diluted by a certain factor by combining the sample with the substrate (e.g., 1:10). In some embodiments, the combination of the sample and the agent is allowed to sit for 1, 2, 3, 4, 5, 6, 7, 8, 9, 10 or more minutes prior to contacting a test cassette with the sample/agent combination. In some such embodiments, when the sample is applied to a test cassette a reacted and/or unreacted portion of the substrate is measured and used to measure the target that reacted with the substrate. For example, in some embodiments, the target is ADAMTS13, and the substrate is VWF. In some such embodiments, the amount of cleaved VWF is measured, which is used to determine the percent activity of ADAMTS13 in a sample.

In some embodiments, a sample is divided, and one portion is pre-processed and measured, and another portion is measured without pre-processing.

In some embodiments, assays of the present disclosure may be improved on one or more characteristics such as efficiency, specificity, sensitivity, accuracy, reliability and/or, latency between obtaining a sample and measuring a target, etc. as compared to one or more previously available assays. In some embodiments, previously available assays comprise enzyme-based assays, radioassays, colorimetric assays, radioimmunodiffusion assays, and/or one or more combinations of assay types. In some embodiments, technologies (e.g., assays, e.g., lateral flow assays) of the present disclosure are improved relative to those described in U.S. Pat. No. 8,865,164 or U.S. Patent Application Publication 2012/0141457, the disclosures of each of which is hereby incorporated in their entirety.

In some embodiments, technologies of the present disclosure (e.g., methods, devices, etc.) allow for rapid measurement of one or more targets. In some such embodiments, such technologies avoid problems common in many previously available assays such as ability to detect and/or quantify appropriate ranges for one or more targets.

Furthermore, and in contrast to previously available assays, in some embodiments, assays described herein are amenable to point-of-care use. For instance, as described herein, the present disclosure provides technologies that require fewer steps and faster results than previously available assays. That is, assays described herein do not require extensive handling (e.g., multiple dilution or pre-processing steps), long incubation times before measurements are made, and/or high numbers of personnel or machinery to measure a target in a sample.

In support of point-of-care use, in some embodiments one or more targets is measured within approximately 30 minutes (e.g., within 20 minutes, within 10 minutes, within 5 minutes) of collecting a sample from a subject. In some embodiments, one or more targets is measured within approximately 35, 40, 45, 50, 55, or 60 minutes of collecting a sample from a subject.

### Assay Components

The present disclosure provides, among other things, assays to measure one or more targets in a sample. Among other things, assay components may be or comprise an immunoassay device as described herein and a reader system (e.g., an immunoassay device reader) upon which a test cassette comprising a sample is loaded and results are read and/or interpreted.

### Immunoassay Device

Among other things, the present disclosure provides immunoassay devices, methods of manufacturing, methods of use, and methods of characterization thereof. As described herein an immunoassay device of the present disclosure is or comprises one or more test strips. In some embodiments, an immunoassay device further comprises at least one additional component. In some embodiments, the at least one additional component is or comprises a sample pad and/or a conjugate pad. In some embodiments, at least one test strip and/or one or more additional components is housed, at least partially, within a test cassette.

In some embodiments, one or more additional solid phases is/are in contact and/or close to a test strip. For example, in some embodiments, a test strip is placed upon a solid backing card. In some embodiments, a clear overlaminate material is placed on top of a test strip. In some embodiments, a test cassette or test cartridge comprises a test strip, which is placed upon a backing card and covered by an optically transparent overlaminate material. In some embodiments, an immunoassay device also comprises a conjugate pad and/or a sample pad. In some such embodiments, a conjugate pad and/or sample pad is/are apposed to or in contact with a test strip. In some embodiments, a test strip of the immunoassay device is contained, partially or completely, within a test cassette or test cartridge. In some such embodiments, the test cassette or test cartridge may be inserted into a reader system (e.g., immunoassay device reader), which reader system is used to measure one or more samples on one or more test strips and test lines of the immunoassay device. In some such embodiments, the reader system may also, for example, comprise means to adjust positioning and measurement processes according to algorithms used to operate one or more components of the system or, the reader system as a whole.

### Test Strip

An immunoassay device of the present disclosure comprises one or more test strips. A test strip is or comprises a solid phase and at least one test line. In some embodiments, a device comprises at least one, two, three, four, five, six or more test strips. In some embodiments, a test strip is or comprises a solid phase. In some such embodiments, the solid phase is or comprises a permeable membrane. In some embodiments, the permeable membrane is or comprises a nitrocellulose membrane. In some embodiments, a nitrocellulose membrane may have a small, medium, and/or large pore size, which pore sizes are associated with particular flow rates such that the larger the pore, the faster the flow rate through the membrane. Slow, medium, and fast pore sizes defined using a water flow rate of 4 cm/second. In some embodiments, a test strip as described herein is contacted by a sample and the sample travels into and through the solid phase of the test strip, flowing laterally using capillary and/or gravitational mechanisms. In some such embodiments, after a sample is applied and flows through the test strip, the sample contacts at least one test line, which test line comprises a capture agent. In some such embodiments, the target is associated with a detecting agent and the capture agent retains the target: detecting agent complex at the test line, where it can later be used to measure the target.

### Test Lines

As described herein, a test strip of the present disclosure comprises one or more test lines. Such test lines act as capture zones for one or more targets from a sample applied to a test strip. In some embodiments, a test strip comprises at least one, two, three, four, five, six or more test lines. In some embodiments, a test line functions as a control line. That is, in some such embodiments, a test line is used to measure a control target of known quantity in a control sample.

In some embodiments, prior to a target being bound, a test line is or comprises one or more capture agents. In some embodiments, a test line is comprised of one or more capture reagents and/or one or more detecting agents (e.g., when a target is captured, and that target comprises a detecting agent). In some embodiments, a test line comprises one or more capture agents and no detecting agents. In some embodiments, a test line comprises one or more detecting agents (e.g., associated with a capture agent on a test line) and one or more capture agents.

In some embodiments, in a test strip comprising more than one test line, all test lines comprise the same capture and/or detecting agents. In some embodiments, in a test strip comprising more than one test line, each test line may comprise different capture and/or detecting agents. For example, in some embodiments, a test line on a given test strip has an equal concentration and volume of one or more components (e.g., capture agents) as compared to another test line on the same test strip. In some embodiments, a test line on a given test strip has a different concentration of one or more components (e.g., capture agent, competing agent) as compared to another test line on the same test strip.

In some embodiments, more than one target may be measured on any single test line. In some embodiments, all test lines on a test strip fit within an optically clear visible window on a test cassette. In some such embodiments, the test lines within the optically clear window are positioned such that a reader system (e.g., an immunoassay reader system) as disclosed herein can visualize and measure one or more targets on one or more test lines.

In some embodiments, a series of test lines includes lines of specific widths, spaced at regular intervals, as described in the present disclosure. In some embodiments, each test line that is part of a series of test lines to measure a given target may comprise the same or different agents, depending upon a given target, and such differences will be clear to those of skill in the art given context of a particular target. In some embodiments, where a test strip comprises more than one test line, such test lines are spaced 2-3 mm apart. In some embodiments, each test line occupies approximately 1 mm×5 mm and is printed at 0.7 μl/cm. It was determined that lines of greater volume produce a gradient of binding that is not compatible with reflectance-based reader methods. In some embodiments, after test lines are striped, the test strip is air dried. In some embodiments, air drying may occur at an elevated temperature (e.g., 37 degrees Celsius). In some embodiments, air drying may occur in a fan oven.

In embodiments comprising a test strip with multiple test lines, one or more test lines closest to the first sample contact point of the test strip comprises a capture agent and is followed by one or more test lines comprising additional capture agent but located further from the first sample contact point. For example, in some embodiments, each test line on a given test strip may be part of a series of test lines designed to measure a given target. In some such embodiments, each test line comprises the same agents, but each test line may serve a different function and purpose within the assay. For instance, by way of non-limiting example, in a series of four test lines, the test line closest to the sample contact and entry point on the test strip may comprise a capture agent and not be used to measure a target, whereas subsequent test lines may comprise detecting agent used to measure a target (i.e., a capture agent binding to a target comprising a detecting agent, which is measured as provided herein). In some such embodiments, the “first” test line can serve to “dilute” the sample and remove an amount of a target that could oversaturate a detectable signal and provide inaccurate measurements.

As described herein, a test line may be or function as a control line. In some such embodiments, such an internal control line verifies whether assay components have functioned properly.

### Capture Agent

In some embodiments a test line of the present disclosure comprises at least one capture agent. According to various embodiments, any of a variety of capture agents may be used to retain a target at a test line. In some such embodiments, at least one test line on the test strip comprises a capture agent. In some such embodiments, a capture agent is associated with a target which itself is associated with a detecting agent, thus a detectable target is retained at a test line comprising a given capture agent. In some embodiments, a target may itself be a detector agent; for example, in some embodiments, a test line may be a control line and a control line may comprise a capture agent that is specific for a detector agent, but not for a target being measured in a sample. That is, in some embodiments, a capture agent binds to a target from a sample (e.g., a target-detector agent complex); in some embodiments, a capture agent binds to a target from the immunoassay device (e.g., a detector agent).

In some embodiments, a capture agent may be one or more antibodies (e.g., monoclonal or polyclonal), antibody fragments, quantum dots, polypeptides, peptides, peptide-like agents, complement receptors or binding proteins, enzymes (e.g., enzyme-based colorimetric detecting agents), arrays, aptamers, bead-based assay related agents (e.g., polystyrene beads), nanodrops, and/or nanoparticles (e.g., gold colloid, e.g., polystyrene, etc.).

In some embodiments, an immunoassay device comprises at least one, two, three, four, five, six or more capture agents per test strip. In some such embodiments, at least one test strip comprises at least one, two, three, four, five, six, or more capture agents. In some embodiments, a single test line comprises a single capture agent. In some embodiments, an immunoassay device of the present disclosure comprises more than one test line and a capture agent may be the same or different on different test lines. For example, in a given test strip with four test lines, each test line may have the same and/or different capture agents.

### Detecting Agents

In immunoassay devices of the present disclosure one or more detecting agents is present on a conjugate pad. In some embodiments an immunoassay device and/or one or more test strips therein comprises at least one detecting agent (i.e., when a target comprises a detecting agent and is captured by a capture agent). In some such embodiments, at least one test line on at least one test strip comprises a detecting agent (e.g., comprises a capture agent binding to a target, which target comprises a detecting agent). According to various embodiments, any of a variety of detecting agents may be used to measure one or more targets. In some embodiments, a test line is measured qualitatively and/or quantitatively. In some embodiments, a test line is not measured (i.e., even if visualizable). In some embodiments, measuring may be achieved through visualizable means including but not limited to colorimetric and/or fluorescent detection. As will be understood by those of skill in the art, visualizable is not limited to detection on a visible spectrum, but includes, but is not limited to, detection in ultraviolet and infrared spectra. In some such embodiments, detection may occur by observation of someone performing an assay. In some such embodiments, detection may occur using a device capable of reading a test strip.

In some embodiments, a detecting agent is provided or utilized alone. In some embodiments, a detecting agent is provided and/or utilized in association with (e.g., joined to) another agent. In some embodiments, a detecting agent may be one or more visualizable or otherwise detectable antibodies (e.g., monoclonal or polyclonal), antibody fragments, quantum dots, polypeptides, peptides, peptide-like agents, complement receptors or binding proteins, enzymes (e.g., enzyme-based colorimetric detecting agents), arrays, aptamers, bead-based assay related agents (e.g., polystyrene beads), nanodrops, nanoparticles (e.g., gold colloid, e.g., polystyrene, etc.), and/or agents for use in photonic crystal enhanced fluorescence (PCEF) assays. In some embodiments, a detecting agent comprises an antibody or portion thereof. In some embodiments, a detecting agent is or comprises an antibody associated with one of more of the exemplary detecting agents as described herein. In some embodiments, a detecting agent is target specific (e.g., conjugated to a target-specific binding agent).

In some embodiments, a detecting agent is or comprises one or more solid or semi-solid structures. In some embodiments, a solid structure is a sphere (e.g., solid or semi-solid nanoparticle). In some embodiments, such nanoparticles may be made of any material known to those of skill in the art, given the context (e.g., gold, polystyrene, etc.) and coupled to or associated with one or more additional detecting agents (e.g., a fluorescent molecule, enzyme, etc.). In some such embodiments, a nanoparticle may be between 20 nm-1000 nm. In some embodiments, a nanoparticle may be between about 20 nm-about 600 nm in diameter. For instance, in some embodiments, a detecting agent may be coupled to or associated with a bead that is approximately 20 nm, 30 nm, 40 nm, 50 nm, 60 nm, 70 nm, 80 nm, 90 nm, 100 nm, 150 nm, 200 nm, 250 nm, 300 nm, 350 nm, 400 nm, 450 nm, 500 nm, 550 nm 600 nm or more.

By way of non-limiting example, a detecting agent may be or comprise various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), nanoparticles (e.g., gold, silver, copper, platinum, polystyrene, etc.) nanoclusters, paramagnetic metal ions, enzymes (for example, enzymes that can be detected with a visualizable label, e.g., chemiluminescent detection agents, colorimetric detection agents, etc.), colorimetric labels (such as, for example, dyes, colloidal gold and/or silver, and the like), biotin, dioxigenin, haptens, and proteins for which antisera or monoclonal antibodies are available.

In some embodiments, an immunoassay device comprises at least one, two, three, four, five, six or more individual detecting agents per test strip. For example, in some such embodiments, at least one test strip comprises at least one, two, three, four, five, six, or more detecting agents.

In some embodiments, detecting agents are used to make one or more qualitative target measurements. In some embodiments, detecting agents are used to make one or more quantitative target measurements. In some embodiments, detecting agents are used to make one or more qualitative and quantitative target measurements.

### Competing Agent

In some embodiments a test strip of the present disclosure comprises at least one competing agent. According to various embodiments, any of a variety of competing agents may be used to adjust a concentration of a target in a sample within a test strip. In some such embodiments, at least one test line on the test strip comprises a detecting agent. In some embodiments, a test strip may comprise competing agent that is not localized to a test line. For instance, in some embodiments, a competing agent may be present in/on a solid phase of an immunoassay device of the present disclosure (e.g., sample pad, conjugate pad, test strip, and/or test line). For example, in some embodiments, a solid phase component of the disclosure may comprise a competing agent to remove a portion of a target. In some such embodiments, a portion of a target may be removed to allow subsequent test lines to efficiently, accurately, specifically, reliably, sensitively and/or rapidly measure a given target in a sample. That is, as will be known to those of skill in the art, previously available assays were unable to measure certain targets without performing serial sample dilutions due to high abundance target concentration oversaturating detectable signal components.

In some embodiments, a competing agent may be one or more antibodies (e.g., monoclonal or polyclonal), antibody fragments, quantum dots, polypeptides, peptides, peptide-like agents, complement receptors or binding proteins, enzymes (e.g., enzyme-based colorimetric detecting agents), arrays, aptamers, bead-based assay related agents (e.g., polystyrene beads), nanodrops, and/or nanoparticles (e.g., gold colloid, e.g., polystyrene, etc.).

In some embodiments, an immunoassay device comprises at least one, two, three, four, five, six or more competing agents. In some such embodiments, at least one test strip comprises at least one, two, three, four, five, six, or more competing agents.

In some embodiments, a competing agent is used to reduce or eliminate a prozone effect. As is known to those of skill in the art, a prozone effect (also known as a hook effect) is a phenomenon that occurs when a concentration of a target is so high that it impairs a binding partner of that target from forming a complex. For instance, in some embodiments, a level of a target may be extremely high such that an antibody to that target is actually impaired from complexing with that target. That is, rather than a linear or otherwise proportionally increasing trend of increased concentration showing increased complex formation, when a prozone effect occurs, as concentration increases, complex formation first stops increasing and then, at very high concentrations, decreases. In some embodiments, a prozone effect may occur, for example, in presence of high concentrations of a target and/or high concentrations of a competing or detecting agent that binds to the target. Accordingly, a competing agent may be added to a sample to reduce concentration of a target prior to measurement of the target by binding or complexing with a detecting agent.

### Solid Phase

In some embodiments, an immunoassay device of the present disclosure comprises one or more solid phases. For example, in some embodiments, an immunoassay device comprises a test strip that is or comprises a solid phase. As described herein, in some embodiments, a test strip comprises a permeable membrane. In some such embodiments, the permeable membrane is or comprises nitrocellulose. In addition to a test strip, an immunoassay device of the present disclosure may further comprise one or more additional solid phases including but not limited to a backing card upon which a test strip sits, a sample pad, a conjugate pad, and/or an overlay material (e.g., an optically clear overlay that covers a test strip).

### Sample Pad

In some embodiments a test cassette of the present disclosure comprises a sample pad. In some embodiments, a sample pad is or comprises a capture zone. In some such embodiments, a sample pad comprises one or more components to modify or alter contents of one or more samples (e.g., by adjusting or buffering pH of a sample). For example, in some embodiments a sample pad comprises a sample buffer. In some embodiments, a sample buffer is specific for a given sample type (e.g., blood, urine, plasma, etc.) to ensure that pH of a liquid sample applied to a test cassette is within a particular range.

In some such embodiments, a sample pad comprises one or more components to modify or alter contents of one or more samples (e.g., by capturing one or more components) before entry onto a test strip. For example, in some embodiments, a sample comprises an antibody. By way of non-limiting example, in some embodiments, the antibody is an antibody to capture and/or filter red blood cells from a whole blood sample after contacting at least a first component of the immunoassay device of the present disclosure, which test strip is not a first component, and prior to the sample contacting at least one test strip of the immunoassay device. That is, a sample may be applied to a sample pad comprising an anti-RBC antibody, such that a sample is depleted of RBCs prior to the sample contacting a test strip of the immunoassay device.

### Conjugate Pad

In some embodiments a test cassette of the present disclosure comprises a conjugate pad. In some embodiments, a conjugate pad is or comprises a solid phase. In some embodiments, a conjugate pad comprises at least one detecting agent. In some embodiments, a detecting agent is or comprises one or more detector beads. In some embodiments, detector beads may be gold or polystyrene. In some embodiments, detector beads may be approximately 30 nm-600 nm in diameter. In some embodiments, a detecting agent is or comprises non-bead based visualizable signal (e.g., fluorescent moieties, etc.). In some embodiments, a conjugate pad comprises two, three, four, five, six or more detecting agents. In some embodiments, a conjugate pad comprises one or more of detergent, one or more buffering components, and/or blocking proteins.

### Housing (Test Cassette/Test Cartridge)

In some embodiments a test cassette comprises an immunoassay device of the present disclosure that is fully or partially contained within a housing. In some embodiments, the solid such a test cassette is or comprises a substantially solid material (e.g., rigid, semi-rigid, flexible), which material surrounds, at least partially, an immunoassay device.

In some embodiments, a test cassette is a three-dimensional shape that may be rectangular, or square.

In some embodiments, a test cassette comprises a port. In some such embodiments, a sample is applied to the test cassette using the port.

In some embodiments, the test cassette may be inserted into a reader system such that a sample flows in a direction of gravity. In some embodiments, a sample is applied to a test cassette and allowed to sit on a surface to develop prior to being placed into a reader system; in some such embodiments, a sample flows through the test strip laterally using capillary action.

In some embodiments, a sample is applied to a test cassette and placed vertically to develop with the sample flowing laterally (using capillary action and gravity). In some embodiments, a test cassette is inserted into a reader system in a vertical position. In some embodiments, a test cassette is inserted into a reader system and a sample is applied to the sample port after insertion; the test cassette is then allowed to develop (i.e., the sample is allowed to travel through the test strip in the direction of gravity) prior to measuring one or more targets on the test strip.

In some embodiments, a test cassette comprises an immunoassay device, which immunoassay device further comprises a sample pad, conjugate pad, backing card (upon which one or more test strips sits), and an optical overlay (covering the test strips comprising the one or more test lines). In some embodiments, the solid material is rigid, semi-rigid, or flexible.

In some such embodiments, a test cassette as disclosed herein is able to be inserted into a reader system. In some embodiments, the test cassette comprises a single port (e.g., RapiPlex, Comp act, etc.). The present disclosure also provides methods of use of such immunoassay devices on one or more reader systems (e.g., RapiPlex, e.g., Comp act, e.g., other immunoassay reader systems). Exemplary reader systems are disclosed, for example, in WO 2013/014540, which is herein incorporated by reference in its entirety.

### Assay Steps

In some embodiments, technologies of the present disclosure provide methods and devices to measure one or more targets using one or more assays as described herein. In some embodiments, an assay comprises one or more steps including obtaining a sample, optionally reserving a portion of a sample (e.g., for later comparison or banking), optionally pre-processing a sample or portion thereof, measuring one or more targets in the sample and, optionally, treating a subject based upon the results of the measuring. In some such embodiments, such methods are performed using assays and devices of the present disclosure. Accordingly, in some embodiments, methods of the present disclosure may comprise steps of obtaining a sample and applying a portion of the sample to immunoassay device of the present disclosure.

After application of a sample, the sample rehydrates at least one test strip of the immunoassay device. In some such embodiments, the sample rehydrates a detecting agent on the test strip (e.g., on the strip, e.g., within test lines). In some embodiments, a target in a sample reacts with a detecting agent, including labeled detector that was added during striping and cassette assembly.

In some embodiments, when a sample comprises whole blood, red blood cells are retained in a filter pad. In some such embodiments, retention is achieved using an antibody (e.g., an anti-RBC antibody) that is applied to or included in a sample pad of a test cassette.

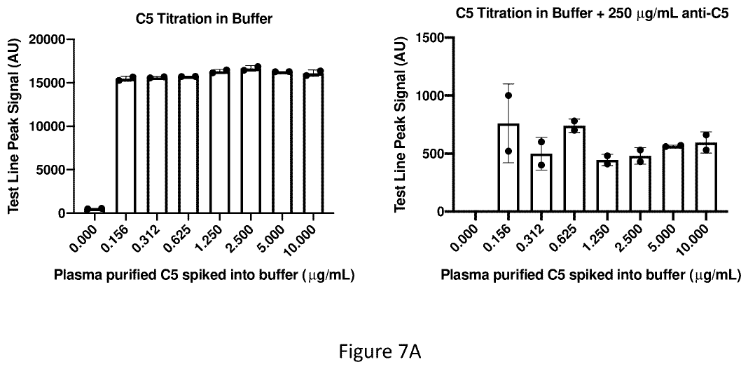

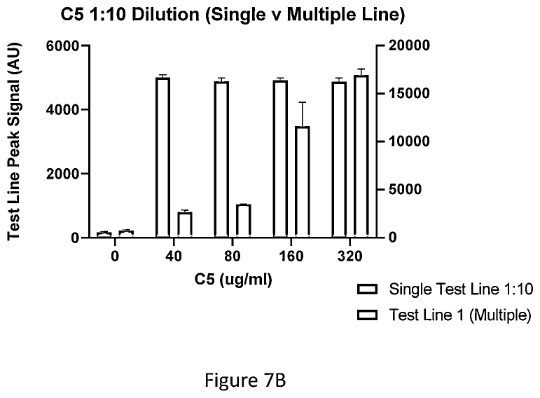

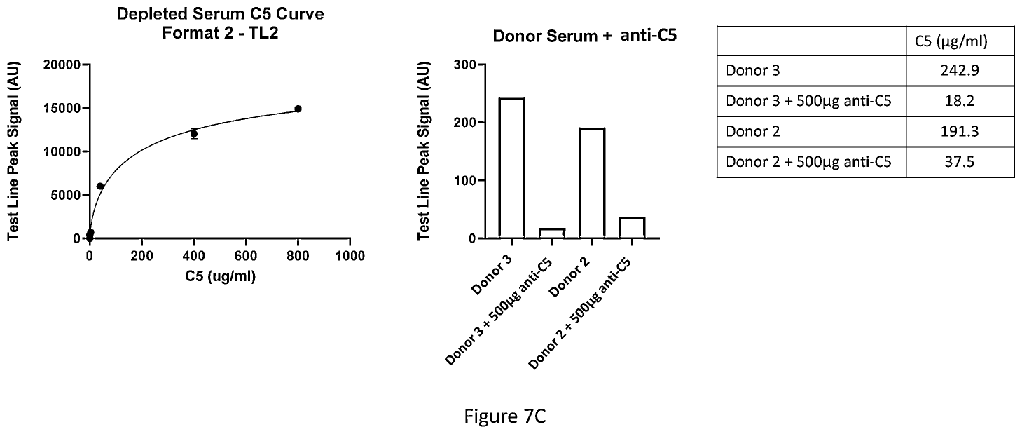

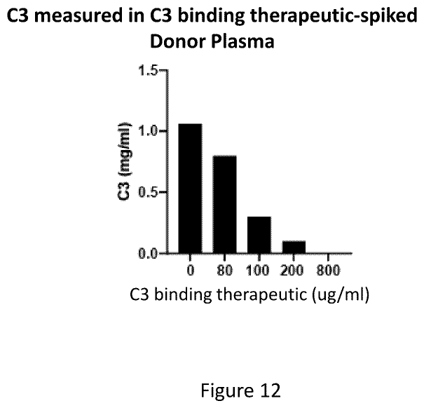

In some embodiments, an amount of a target interacts and reacts with a competing agent to remove that amount of target from the pool of “free target” in a sample. After removal of an amount of target at one point of the test strip (e.g., closer to the application port where the sample is initially applied), the sample continues flowing through the solid phase of the immunoassay device. For instance, by way of non-limiting example, FIGS. 7 and 12 depict an exemplary competing agent removing a portion of a target from a sample. In some such embodiments, a target is considered a high abundance target. In some such embodiments, after a sample has contacted a competing agent, a complex of target-competing agent may continue to migrate onto and along at least one test strip but will move over and past any subsequent test lines allowing any additional “free target” to bind/be captured by any detection agent on those test lines for measurement in accordance with the present disclosure.

In some embodiments, where a conjugate pad comprises a competing agent and a target binds to a competing agent, upon entry into a test strip, any target: competing agent complexes will migrate past a test line comprising a capture agent that binds the same target as the competing agent. In some embodiments, where a target is bound to a detecting agent, when a sample migrates to and through a test line, a capture agent may bind to the target such that a capture agent is associated with a target which is associated with a detecting agent and can then be visualized.

In some embodiments, a test strip is left to sit for 5, 10, 15, 20, 25, or 30 minutes for an assay to develop (e.g., for one or more targets in a sample to interact and react with the test strip and one or more detecting and or competing agents). In some embodiments, upon development, a test cassette is inserted into a reader system (see, e.g., WO 2013/014540 for exemplary reader systems) and a test protocol is selected. In some embodiments, a sample is added to a test cassette after it has been inserted into a reader system and the development is allowed to occur with the test cassette already positioned in the test reader. In some embodiments, a test cassette comprises a bar code that is read by a reader system. In some embodiments, a user must select a test protocol that corresponds to the test sample and test panel of the one or more test strips of the assay. In some embodiments, a test cassette is placed into an appropriate area on a reader system, sample identification entered or automatically uploaded (e.g., in barcoded cassettes), and a scan of the one or more test strips is performed and one or more targets is measured.

In some embodiments, a QC cassette is run before or after a test cassette to confirm that a reader system is operating within a set of acceptable parameters, as will be understood by those in the art using such reader systems.

In some embodiments, results from one or more measurements is/are memorialized on a local device, on a server, and/or in another recordable and saved medium. In some such embodiments, results are recorded in a patient record.

Samples may be detected using detectors that detect one channel or multiple channels (see, e.g., Comp act or RapiPlex reader systems, e.g., U.S. Pat. No. 9,199,232, which is incorporated herein by reference in its entirety; see also, e.g., WO2013/014540 for description of cartridges, systems, devices, and reader systems).

### Measurement

In some embodiments, a measurement is quantitative. In some embodiments, a measurement is qualitative. In some embodiments, a measurement includes a qualitative assessment of “absent.” In some embodiments, a measurement includes a quantitative assessment of “zero” or below a LLOQ.

In some such embodiments, a quantitative measurement of a target is expressed as a percent activity of a reference. For example, in some embodiments, a target is ADAMTS13, and a measurement is expressed by determining percent activity using a known concentration of a starting VWF substrate in a mixture of sample and substrate.

In some embodiments, a target is measured indirectly, such as through reaction with a substrate and quantification of substrate consumption, cleavage (e.g., of substrate) or other detectable modification.

In some embodiments, a measurement is qualitative. In some embodiments, a measurement includes detecting absence of a target.

In some such embodiments, an amount of a target (e.g., concentration) is zero. In some embodiments, an amount of a target appears to be zero, but may, in some embodiments, be present in low levels but below the LLOQ of measurement. In some embodiments, a measurement includes detecting presence and/or amount (e.g., concentration) of a target.

### Methods of Use

The present disclosure provides technologies, including methods of use of devices. In some embodiments, the present disclosure provides methods of characterizing and/or measuring a target as well as methods of diagnosis, monitoring, and treatment of a patient as disclosed herein. In some embodiments, as subject has or is at risk of having or developing one or more diseases, disorders or conditions as disclosed herein. In some embodiments, a subject is receiving treatment with, e.g., a therapeutic agent (e.g., antibody, e.g., gene therapy, e.g., CAR-T, etc.).

### Diagnosis, Treatment and Patient Monitoring

Among other things, the present disclosure provides, a variety of new methods and treatment regimen for improving treatment of a patient with one or more complement-mediated or related diseases, disorders, or conditions as described herein. In some embodiments, the present disclosure provides methods of diagnosis for patients suffering from or at risk of one or more diseases, disorders or conditions as described herein. In some such embodiments, such methods of diagnosis comprise excluding one or more diseases, disorders, or conditions from a list of suspected or differential diagnoses in a patient.

In some embodiments, the present disclosure provides methods of treatment for patients with one or more diseases, disorders or conditions as described herein. In some embodiments, patients may be suffering from or at risk of two or more diseases, disorders, or conditions.

In some embodiments, a treatment describes any administration of a procedure, intervention, substance or any combination thereof that partially or completely alleviates, ameliorates, relives, inhibits, delays onset of, reduces severity of, and/or reduces incidence of one or more symptoms, features, and/or causes of a particular disease, disorder, and/or condition. For example, in some embodiments, a treatment may comprise an intervention that includes withdrawal or removal of a substance (e.g., from a patient). In some embodiments, administration of a treatment may comprise removal of a composition and/or addition of a different composition and/or intervention. In some embodiments, administration comprising removal of a composition may be or comprise adding an intervention such as plasma exchange (e.g., to remove or dilute a particular agent in the system of a subject). In some embodiments, treatment may be of a subject who does not exhibit overt signs of the relevant disease, disorder and/or condition; for example, in some embodiments, treatment may be administered when one or more changes in one or more targets is detected, regardless of whether any overt clinically observable phenotype occurs (i.e., other than measurements of targets). In some embodiments, treatment may be of a subject who exhibits only early signs of the disease, disorder, and/or condition. Alternatively or additionally, in some embodiments, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

As described herein, a subject may have or be at risk of having one or more diseases, disorders, or conditions associated with changes in a complement protein or complement-pathway associated protein. For instance, by way of non-limiting example, in some embodiments, a subject may have or be at risk of having or developing Hematopoietic Stem Cell Transplant-Associated Thrombotic Microangiopathy (HSCT-TMA), Complement-Mediated Thrombotic Microangiopathy (CM-TMA), atypical hemolytic uremic syndrome (aHUS), thrombotic thrombocytopenia purpura (TTP), COVID19, lupus erythematosus, lupus nephritis, cytokine release syndrome, Alzheimer's Disease (AD), or combinations or complications thereof.

In some embodiments, the present disclosure provides methods for monitoring disease activity of a patient with or at risk of one or more diseases, disorders, or conditions as described herein. In some embodiments, the present disclosure provides methods for predicting onset or increase in activity (e.g., a disease flare, e.g., increase in one or more symptoms of a disease) of a patient with or at risk of one or more diseases, disorders, or conditions as described herein. In some such embodiments, such methods provide for improved treatment as compared to treatment in absence of such technologies (e.g., devices, methods) as provided by the present disclosure.

In some embodiments, the present disclosure provides methods for diagnosing a subject with a disease, disorder or condition. For example, without being bound by any particular theory, in some embodiments, the present disclosure provides technologies for measuring one or more levels of one or more targets and, upon comparison of measurements of those targets such as to a control/external reference or to a previous sample from the same patient, a diagnosis of a disease, disorder or condition is made or excluded. In some embodiments, such a disease, disorder, or condition may be or comprise age-related macular degeneration (AMD), complement 3 glomerulopathy (C3G), Hematopoietic Stem Cell Transplant-Associated Thrombotic Microangiopathy (HSCT-TMA), Complement-Mediated Thrombotic Microangiopathy (CM-TMA), atypical hemolytic uremic syndrome (aHUS), thrombotic thrombocytopenia purpura (TTP), COVID19, lupus erythematosus, lupus nephritis, cytokine release syndrome, Alzheimer's Disease (AD), or combinations thereof.

In some embodiments, the present disclosure provides methods for preventing or reducing the severity of one or more symptoms associated with risk of developing or diagnosis of one or more diseases, disorders or conditions as provided herein. In some such embodiments, for example, the present disclosure provides a method of administering a treatment or implementing a change in treatment if a measurement of one or more targets is outside of a particular range as disclosed herein.

In some embodiments, the present disclosure provides methods of determining the effectiveness of a therapy for treating at least one disease, disorder or condition. For example, in some embodiments, the present disclosure provides methods including a step of determining in a sample from a subject at risk of having, suspected of having, or having one or more diseases, disorders, or conditions as described herein, a first target measurement, and optionally administering at least one treatment to the subject if a target measurement does not fall into a satisfactory range. In some embodiments, the present disclosure provides methods for, if a treatment is administered to a subject, determining in a sample from the same subject, a second target measurement (of the same or a different target as the first measurement), and implementing a change in treatment if the second measurement does not fall within a satisfactory range.

In some embodiments, administering or implementing a change in treatment results in an increase in measurement of one or more targets in the subject. In some such embodiments, the subject does not experience an increase in risk of disease or one or more symptoms of disease within approximately 30 minutes, 1 hour, 2 hours, 3 hours, 4 hours, 5 hours, 6 hours, 7 hours, 8 hours, 9 hours, 10 hours, 12 hours, 18 hours, 24 hours, 48 hours, 72 hours, 96 hours, one week, two weeks, three weeks, or four weeks of the administering or implementing a change in treatment.

In some embodiments, administering or implementing a change in treatment results in an increase in measurement of one or more targets in the subject. In some embodiments, the increase in measurement of one or more targets occurs within one month from the administration or implementation step (e.g., within three weeks, within two weeks). In some embodiments, the increase in measurement of one or more targets occurs within one week from the administration or implementation step (e.g., within six days, within five days, within four days, within three days, within two days, within one day).

In some embodiments, administering or implementing a change in treatment results in a decrease in measurement of one or more targets in the subject. In some such embodiments, the subject does not experience a decrease in risk of disease or one or more symptoms of disease within approximately 30 minutes, 1 hour, 2 hours, 3 hours, 4 hours, 5 hours, 6 hours, 7 hours, 8 hours, 9 hours, 10 hours, 12 hours, 18 hours, 24 hours, 48 hours, 72 hours, 96 hours, one week, two weeks, three weeks, or four weeks of the administering or implementing a change in treatment.

In some embodiments, administering or implementing a change in treatment results in a decrease in measurement of one or more targets in the subject. In some embodiments, the decrease in measurement of one or more targets occurs within one month from the administration or implementation step (e.g., within three weeks, within two weeks). In some embodiments, the decrease in measurement of one or more targets occurs within one week from the administration or implementation step (e.g., within six days, within five days, within four days, within three days, within two days, within one day).

In some embodiments, the at least one treatment includes administering or stopping administration of one or more of gene therapy, cell-based therapy, a checkpoint inhibitor, steroids, non-steroidal anti-inflammatory drugs (NSAIDs), hydroxychloroquine, chloroquine, quinacrine, methotrexate, azathioprine, sulfasalazine, cyclophosphamide, chlorambucil, cyclosporine, mycophenolate mofetil, mycophenolate sodium, rituximab, belimumab, complement inhibitors such as one or more anti-complement protein antibodies or other chemical (e.g., small molecule, nucleic acid, etc.) inhibitors thereof, plasmapheresis, physical therapy, sleep therapy, and cognitive behavioral therapy. In some embodiments, the at least one treatment is or comprises part of a combination therapy administered to the subject.

In some embodiments, a treatment is administered to a subject in vivo. In some embodiments, at treatment is administered ex vivo (e.g., to cells or fluids of a subject and then the treated cells or fluids are introduced into the subject). In some embodiments, a treatment is tested or characterized in vitro (e.g., in or comprising a bodily fluid from a subject, in an artificial bodily fluid, in a cell line, in a primary cell or cell culture, etc.).

### Kits

In some embodiments, the present disclosure provides kits to measure one or more targets as disclosed herein. In some embodiments, a kit comprises an immunoassay device housed inside a test cassette and further comprises packaging appropriate for storage. In some embodiments, packaging may be or comprise one or more qualities including light blocking (e.g., a foil bag), desiccant ingredients or components, and thermostable materials to maintain cassettes in ambient, dry conditions (e.g., protected from humidity, etc.).

In some embodiments, a kit comprises a test cassette comprising an immunoassay device which itself comprises up to six test strips, with each strip comprising at least four test lines and, optionally, at least one control line (i.e., up to five test lines, one of which is or functions as a control line).

In some embodiments, a control sample is provided to run on a control test cassette and/or to measure on a control line on a test line of an immunoassay device as provided herein.

In some embodiments, a kit comprises a test cassette comprising a bar code. In some embodiments, a kit of the present disclosure comprises a disposable transfer pipette for applying a sample to the test cassette.

### EQUIVALENTS

It is to be understood that while the disclosure has been described in conjunction with the detailed description thereof, the foregoing description is intended to illustrate and not limit the scope of the present disclosure, which is further defined by the scope of the appended claims. Other aspects, advantages, and modifications are within the scope of the following claims.

### EXAMPLES

The present disclosure provides technologies that can, among other things, efficiently, sensitively, specifically, reliably, rapidly and/or accurately measure (e.g., detect, e.g., quantify) one or more targets in a sample, without having to dilute the sample prior to measurement. That is, technologies described herein can achieve rapidly and reliably achieve and provide accurate measurements of one or more targets across a wide range of concentration(s) without requiring off-line dilutions or other sample manipulation. Such measurements can provide previously unachievable insight and information that can be used, for example, in research and development, as well as clinical trial monitoring and point-of-care diagnostics and/or monitoring of therapeutics in patients. The following Examples demonstrate exemplary devices, targets and methods of rapid and accurate measurement therein, including measurement of binding agent: target interactions and improvements upon existing technologies (see, e.g., Schramm E C et al., (2015) Anal Biochem, 477:78-85; U.S. Pat. Nos. 8,835,184, 9,164,088, and 9,182,396, each of which is herein incorporated by reference in its entirety).

### Example 1: Test Cassette Assembly

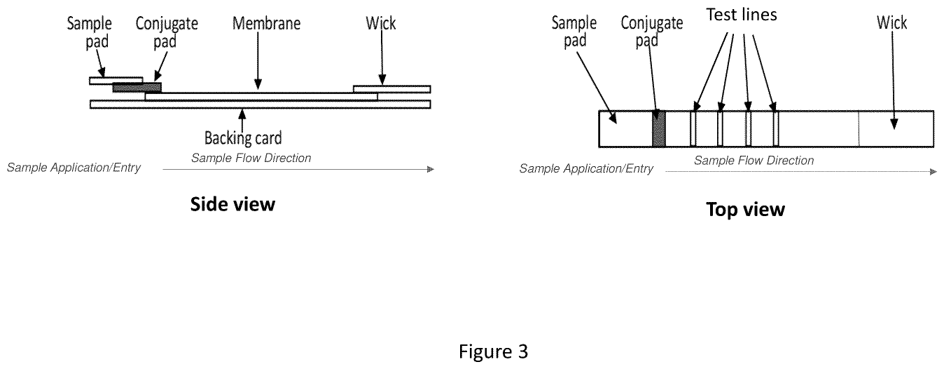

The present example provides assembly of an exemplary cassette comprising an immunoassay device, which can be used with methods provided herein. Such cassettes are compatible with different reader system systems (e.g., Comp act or RapiPlex Systems). A schematic of an exemplary configuration of components housed in a test cassette (e.g., an immunoassay device comprising at least one test strip, which test strip comprises at least one test line; sample pad; conjugate pad; and/or backing card), is shown in FIG. 3.

### Test Cassette Assembly

The test cassettes were manufactured on 30 cm×60 mm or 30 cm×35 mm backing cards for use in Comp act and RapiPlex measuring devices, respectively. In the present Example, single-sided pressure sensitive adhesive polyester was used. The backing cards provide physical structure and support for the cassette and test membrane. A test strip or a set of six test strips comprising a nitrocellulose (NC) membrane was laminated onto a backing card. In some versions, a NC membrane with a larger pore size was used.

### Striping

Unlabeled antibody reagents (i.e., capture agents) were immobilized on a NC membrane in their respective capture zones (i.e., in a test line) using in-line striping equipment to apply test lines. Test lines were applied to test strips using Imagene equipment (see, e.g., U.S. Pat. No. 9,199,232, which is herein incorporated by reference in its entirety). Striping techniques were developed to be able to quantify targets over four to five logs of concentration (improving by at least one to two logs, upon previously available assays) for one or more targets, within one test strip using one sample. That is, the striping techniques developed and described herein were created and designed such that assays of the present disclosure could be performed with more than one type of liquid sample, such as blood or urine, and furthermore, assays could be performed with neat samples or samples that had not been significantly diluted (e.g., samples comprising blood with no more than 1-2× dilution versus previous assays which required 1:20 dilutions).

As will be understood by those of skill in the art, it is not trivial nor straightforward to switch from one type of sample (e.g., blood) to another (e.g., urine) and maintain the same specificity and sensitivity for a target. For example, prior to technologies as provided herein, assays (e.g., ELISA assays, e.g., single line lateral flow assays) for sC5b-9 were able to detect 100-100,000 ng/ml in blood; in addition, such assays required a 1:20 offline dilution. In contrast, technologies as described herein achieve improved lower limits of quantification (e.g., 1 ng/mL for sC5b-9) while simultaneously being able to continue to detect targets at very high upper limits of quantification (e.g., 100,000 ng/ml for sC5b-9) and do so without requiring several serial dilutions, for instance, to get a target in a measurable range for a given assay. Thus, provided technologies also improve assay ranges for one or more targets.

Among other things, this improvement allows samples such as urine, which may have more dilute levels of target, to be measured without further significant offline dilutions which could, for example, increase processing time, disrupt or produce artifacts in measurements of one or more targets, and/or increase costs and steps associated with target detection and quantification. That is, the present disclosure provides assays that are capable of being used with targets in blood or liquid samples other than blood, such as urine, ocular fluid, cerebrospinal fluid, etc. In contrast, other assays are capable of detecting certain targets (e.g., sC5b-9) in blood or urine, but do not have the sensitivity or improved utility of assays described herein and can therefore result in false negative measurements. Furthermore, the technologies provided herein create and also improve upon various features of prior assays such as improved offline dilution ratios, improved lower limit of quantification, and improved sensitivity and accuracy.

Importantly, to overcome limitations of and challenges associated with quantification in assays such as lateral flow assays which may include a single test line to detect a single target, test strips were striped with multiple test lines capable of detecting and quantifying one or more targets. Here, a combination of multiple test lines, competing, capture, and/or detecting agents, and/or one or more agents on a sample and/or conjugate pad provide unexpected and unprecedented efficiency, accuracy, sensitivity, specificity, reliability and/or speed of target measurement. For example, striping a strip with multiple test lines allows measurement of low concentrations of a particular target as well as high concentrations without needing any substantial dilution or manipulation of a sample.

Importantly, these innovations provide ability to measure low and high abundance targets, as well as targets that may be or comprise an unstable protein in contexts where concentration ranges are wide and/or manipulation of a sample could impact whether a measurement is accurate. For example, as described herein, a target that can be activated by manipulation and cause a falsely elevated measurement reported for that target or a target that can be dissociated from a therapeutic and result in a falsely elevated measurement reported for that target. Here, results demonstrate that multiple test lines improve detection ranges of particular targets, which provides a solution to longstanding problems with assay limitations due to narrower detection ranges or saturation. That is, narrow detection ranges in previously available assays were caused from insufficient LLOQ and/or ability to detect low abundance targets and saturation of detection agents results in artifactual measurements. Importantly, in the absence of multiple lines, increasing concentrations of target detector did not improve assay sensitivity. Rather, assay sensitivity was negatively impacted because increased in detector concentration on a test strip with a single test line showed increased background and/or masked test line signals from detection.

Accordingly, in some versions of assays of the technologies of the present disclosure, multiple capture zones are striped for each target per membrane. Each test line (stripe) occupies approximately 1 mm×5 mm and is printed at 0.7 μl/cm to ensure a crisp sharp line. It was determined that lines of greater volume produce a gradient of binding that is not compatible with reflectance-based reader methods. In some versions of the assay, multiple lines are striped for a first protein (for removal) followed by test lines for the target. The control capture reagent is an independent antibody pair and was striped onto each respective membrane in the control zone (downstream of the capture zone).

In one type of test strip, it was determined that 4 mg/mL (˜2 μg of protein per test line per test strip) provides maximum capacity for stable binding of a target detection agent (e.g., an antibody) to nitrocellulose membrane which makes up the strip. It was determined that overloading a printed test line with protein could risk an uncontrolled amount of antibody washing off in an initial sample front passing into and through the test lines which could, in turn, impacting the inter-strip reproducibility, sensitivity and product stability.

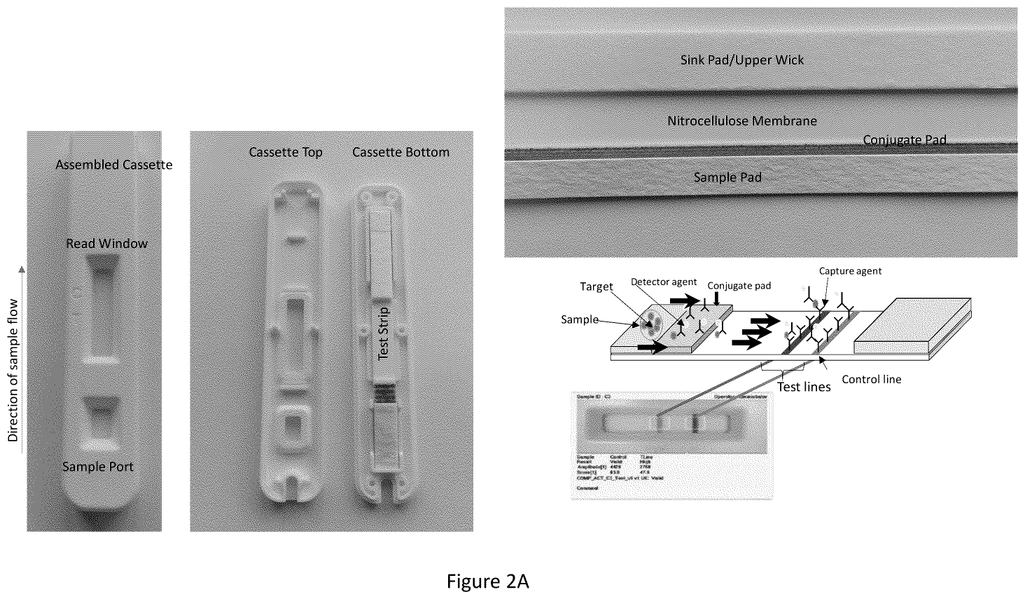

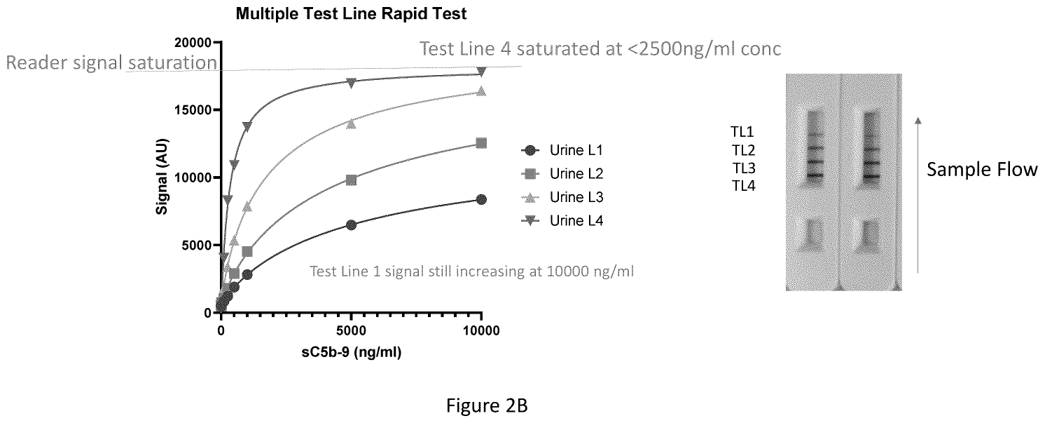

Test strips were developed with at least four test lines (see, e.g., FIGS. 3, 4, and 5) and, optionally, at least one control line per test strip. In one version of the assay, each test line is within the readable window of a cassette (see, e.g., FIG. 2B showing a cassette with four test lines). Test lines were spaced 2-3 mm apart to allow detection on the reader. An internal control verified that test components have functioned properly, and the membrane was then air dried at an elevated temperature in a fan oven.

As shown in, e.g., FIGS. 2B, 3, and 4, the test line closest to the sample port and/or conjugate pad (L4) detects one end of a range of sample (e.g., lowest concentration) and L1, which is furthest from the sample port, towards the end of the strip in direction of sample flow, detects high end concentrations in cases where, e.g., L4 and L3 are saturated. Due to this unique setup and combination of test lines and reagents, test strips and cassettes of the present disclosure are able to efficiently, specifically, accurately, reliably, and/or sensitively measure one or more targets from a sample without prior significant dilution (e.g., neat samples).

### Striping for Non-Specific Antibody Incorporation

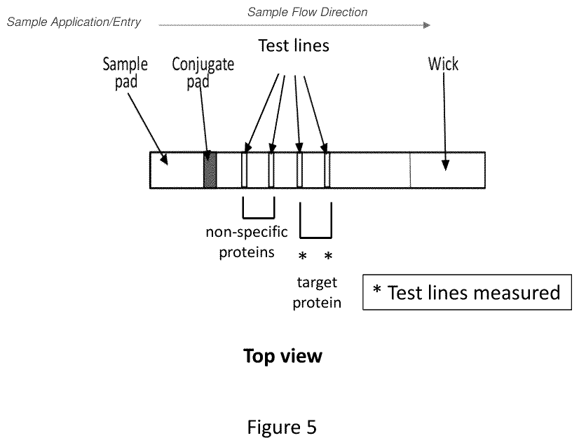

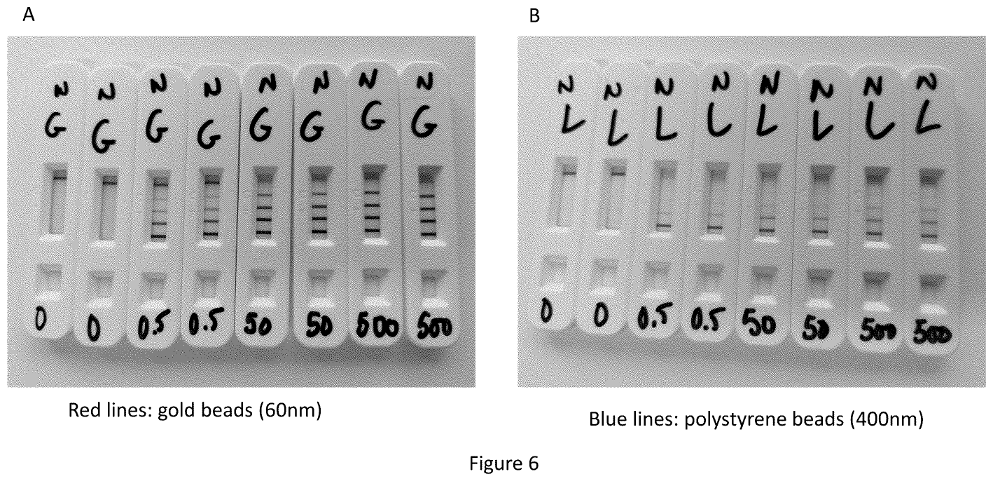

As an example: the first 2 of 4 test lines that a sample will move over are striped containing an antibody to capture excess protein (e.g., anti-Factor B mAb) that is designed to remove that protein. The second 2 lines in order of sample flow will capture the target, Ba. Then, only the final two lines, the target test lines, are measured (see, e.g., FIG. 5).

Striping for Additional Detector (to Remove “Excess” Target from a Sample)

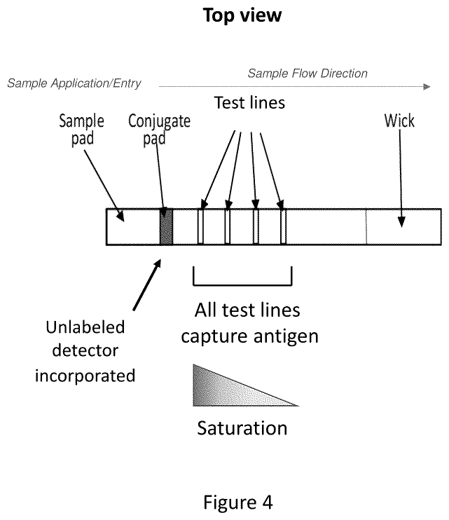

At least 4 test lines are striped for assays involving a high concentration target. The first test lines (e.g., lines 1 and/or 2) will absorb target and become saturated. The remaining target will move over the next test line and if still in excess this test line will also become saturated. This process continues. An accurate reading is obtained from a test line that is not saturated (see, e.g., FIG. 4). An additional step is incorporated for certain targets that is also designed to overcome the issue of high concentration. This step requires competing agent to be incorporated in the conjugate pad. This competing agent absorbs excess target without being visualized as or by a detecting agent and, therefore, saturation of the assay doesn't occur, and accurate measurement can be obtained (see, e.g., U.S. Pat. No. 9,199,232 describing capture zones, including for multiple targets).

### Test Line Application for Multiplexed Test Cassettes

To generate immunoassay devices and test cassettes that are capable of measuring one or more targets from a single sample, test lines are prepared as described herein, with appropriate reagents (e.g., detecting agents, e.g., competing agents, etc.). Test lines are striped comprising one or more capture agents to capture and visualize multiple targets in a given test strip. Immunoassay devices for multiplexing may comprise up to six test strips, each with up to four test lines for a total of twenty-four distinct measurement lines.

### Conjugate Pad Preparation

The conjugate pad was prepared with the labeled detector (see, e.g., FIG. 2A). In some versions of the assay, a detector bead with larger surface area was used. In other versions of an assay as described herein, competing agent was added to the conjugate pad. In still other versions of an assay as described herein, the detector concentration was doubled.

The conjugate pad, sample pad and NC membrane were laminated as a stack onto the backing card (see, e.g., FIGS. 3, 4, and 5). In some versions of assays described herein, an optically clear overlaminate was placed over the test strip portion and a portion of the membrane, covering the free space diffusion zone. The overlaminate was positioned to leave a 3 mm contact zone of exposed upper wick at the distal end of the membrane. The laminated cards were then converted into 5 mm width and the assembly was placed in a cassette. Assembly of the cassette brought the bulk absorbent material into contact with the exposed contact zones of the membranes.

### Improvement of Sample Flow

During assay development, in some tests, when a sample was placed into the test cassette, entry into and/or through the test strip appeared impaired. To improve cassette function for assays where sample movement appeared impaired, the NC solid phase was replaced with a faster flow rate NC membrane that has a larger pore size. After replacement of the membrane, samples flowed into the cassette without getting stuck at any portion of the cassette assembly.

### Example 2A: Assay Development

The present Example describes development of technologies of the present disclosure. In particular, the present disclosure provides insights and unique combinations of steps to create a test strip as described herein, as well as provide for methods of use thereof, such that assays using test strips and immunoassay devices as described herein achieve results with improved efficiency, sensitivity, specificity, reliability, speed, and/or accuracy when compared to other immunoassays. This format also allows multiplexing and use of samples that do not required extensive handling or processing prior to measuring. Assay development and creation of test strips as disclosed herein begins with selection of one or more detection or capture agents. That is, here, an exemplary test strip, such as one that would be part of an immunoassay device of the present disclosure, was used to measure two exemplary targets, C3 and C5, as disclosed herein.

Antibody Selection: To begin, antibody selection was performed using a direction antigen EIA format to screen several antibodies (e.g., commercial antibodies, custom antibodies, etc.). This initial screening process established a baseline sensitivity and specificity of each tested antibody in a neutral sample matrix (here, buffer). Antibodies were then screened against antigen-coated plates to determine ultimate sensitivity (e.g., lower and upper limits of detection) for the target. Antibodies were also screened against cross-reacting antigen-coated plates to determine specificity for the target. Antibody concentration was titrated to compare performance of all antibodies being evaluated.

EIA Assay: Selected antibodies (from screening in Step 1) were evaluated in a sandwich immunoassay. These evaluations were done to confirm that the selected antibodies were complimentary and not competing for one epitope of the target. Each antibody under evaluation was tested in both orientations: (i) on the solid phase; and (ii) as the liquid/fluid-based detector. This established the sensitivity of any pairs of antibodies, but, importantly, the EIA configuration did not always translate to the LFA configuration of a test strip disclosed herein including, e.g., because LFA formats require faster kinetics than EIAs and, thus, greater specificity of target-binding agents including because in contrast to an EIA, non-specific material is not washed away in an LFA format. Here, the setup is similar to a standard immunoassay in that a capture antibody is on the solid phase, target is captured, and liquid detector binds to the captured target.

Half-Dipstick LFA: Each antibody was striped as a single test line onto a mid-flow rate nitrocellulose membrane at 1 mg/mL in buffer with similar salt & pH to the buffer the antibody was originally supplied/rehydrated in. An anti-species antibody complimentary to the detector antibody to be evaluated was striped 5 mm from the test line. Printed test cards were dried for a minimum of 30 minutes at 37 degrees Celsius. If needed, dried cards may be stored sealed in foil pouches with desiccants.

Each antibody was coupled to a detector (gold colloid, polystyrene bead or fluorescent molecule). Ratio of antibody: detector, concentration of antibody at coupling stage, coupling buffer, pH at each stage of coupling process, time for each step and recipe of both the post coupling buffer and storage buffer were assessed in the next steps. Small mL volumes of conjugates were made to assess coupling conditions for antibodies by testing small volumes over a range of various conditions, storing, and retesting to determine appropriate conditions for scale up. The OD and visual clarity of the conjugates were recorded at time of manufacture. Conjugates were stored refrigerated and prior to testing OD and visual clarity, inspected again. Any solutions that have >10% change in OD, change in visual clarity and uniformity of the liquid conjugate is an indication the conjugate recipe is not stable and should not be carried forward for testing. All antibodies were conjugated in absence of SDS, azide and thimerasol.

On day of testing, the prepared conjugate was mixed 1:1 with buffer containing 1% Tween 20. Importantly, this solution was tested within 60 minutes of preparation. Test cards were cut into 5 mm, dipsticks, with only a cellulose upper wick attached (minimum 2 mm overlap). Using a 96 well plate, 20 μL of prepared conjugate was combined with 20 μL of target antigen in buffer. The half dipstick was added and let run to completion (i.e., when the well is dry). The test strips were read on an exemplary device reader system (in this example, Comp act or RapiPlex, depending upon construction of the immunoassay device) which measured test line signals. In addition, strips were inspected to (1) note no “meniscus” line at the sample port, which is a possible indication of conjugate collapse and (2) overall background is not highly stained, which is a possible indication of inadequate detergent, blocking buffer recipe in conjugate.

As in the EIA, here, each antibody was tested both as a Test Line and as a Detector to identify optimal pair. Further testing of sample diluent (salt, pH, detergent and additives), concentration of Test Line, and concentration of detector were also tested to characterize each antibody pairing. At least one pair of antibodies that were detecting the target and showing increase in signal test line that correlates to increase in concentration of antigen in the sample well was identified. The additional rounds of testing chemistry in half dipstick were performed to confirm baseline sensitivity and antibody orientation in an LFA format.

Full LFA: After the half-dipstick, the next step was to integrate/dry down all agents that will be present on the test strip and to create an assay with appropriate volume, test strip material, material lamination and timing as needed for final Product Requirements (e.g., goal ranges for a given assay, known cross-reacting targets, known concentration limits in both health and disease states, etc.).

Preparation of conjugate pads in full LFA: Liquid conjugate preparation was scaled up from the half dipstick step in order to make the conjugate in a volume that is sufficient to concentrate the conjugate stock to a higher concentration to dry into the conjugate pad material in order to achieve appropriate dried concentration such that upon rehydration during assay performance, measurements are made in accordance with the design and parameters as disclosed herein. In the half dipstick step 20 μL of conjugate was used in testing, while in a dry test strip format the materials used to hold the conjugate can only hold approx. 10 μL volume. The liquid conjugate stock including concentrated material should be stored refrigerated. Conjugates were stored refrigerated and prior to testing, OD and visual clarity inspected again. Any solutions that have >10% change in OD, change in visual clarity and uniformity of the liquid conjugate is an indication the conjugate recipe is not stable and should not be carried forward for testing.

Conjugate pad materials were glass fiber or polyester based. Denser materials were not used because upon testing, they did not release all of the conjugate within the required assay time. Polyester mixed materials are hydrophobic and required pre-treatment before spraying conjugate onto the pads. The polyester pads were soaked in excess of buffer containing detergent. An optional component to assay development that was performed at this step was to evaluate pre-soaking conjugate pads glass fiber included to introduce additional detergent, buffering capacity and blocking proteins into the test chemistry (i.e., the set of reagents dried onto the conjugate pad). Conjugates were prepared in a spraying buffer that contains sugars, which was determined to assist with release of conjugate from the pads. Specific sugar and % w/v content were optimized for each assay.

Nitrocellulose Membrane: Nitrocellulose (NCE) membrane was prepared for half-dipstick analysis as described herein. As part of the full test strip parameters including flow rate, placement of lines relative to the sample port, sample pad and conjugate pad dimensions and material density were evaluated. The line positions were located within the “read window” of the detection platform. A minimum of 2 mm spacing between consecutive test lines was used in order to provide enough clearance for proper measurements (e.g., accurate/optimal detection of visualizable assay components). The test lines were printed at no more than 1 μL/cm to achieve sharp crisp test lines.

Other Pads: Sample pad preparation was optimized for each sample type (e.g., whole blood, urine, plasma, etc.). For use with blood samples, a means for capturing red blood cells (e.g., an anti-RBC antibody) was added. For use with urine, buffering capacity was adjusted to normalize urine pH and filter any cell debris and/or protein that may be present in a urine sample. Upper Wick is a sink pad laminated onto the test strip furthest from the sample port and was designed to have material and overlap onto a NCE membrane such that flow rate of a sample (e.g., blood, e.g., urine) was maximized through a test strip while providing a means for detector to be cleared out to maximize test line signal emission.

Final sample matrix configuration: Test reagents, nitrocellulose membrane flow rate, sample volume and pad chemistries were all tested and then optimized in an iterative manner to maximize the signal: noise and sensitivity of the tests.

Once a test configuration was developed the next stage was to verify that performance of the test was reproducible across multiple raw material preparations.

### Example 2B: Assay Method

A sample was applied into the sample port of the test cassette using the supplied disposable transfer pipet. Timing is very important, and samples should be applied to a cassette as quickly as possible. Specifically, within one hour of collection and no more than 5 hours, with samples held on ice until application to cassette. Amount of time a sample is at ambient temperature should be very limited to ensure accuracy of measurements/readings. If testing is not available for freshly isolated samples, samples may be measured after storage if sample handling/workflow is optimized prior to and during storage. This includes minimal time at room temperature, quick processing of sample (e.g., into to plasma), and transfer to storage (−80 degrees Celsius) until ready to assess on a cassette as described herein. Upon thawing, a previously frozen sample (e.g., plasma, urine, saliva, etc.) should be read immediately.

After application, the sample rehydrated the strip, including labeled detector that was added during striping and cassette assembly. A target in the sample reacted with labeled detector. In some versions of an assay where a sample comprises whole blood, red blood cells were retained in the sample pad by, e.g., using an anti-RBC antibody that is applied or included in the sample pad. In other versions, the “excess” target, which is a portion of a target that, if present, would saturate a signal and exceed the ULOQ of a test line, reacted with competing agent to remove the “excess” from the sample. The complex of target-detector then migrated onto the NC solid phase and moved over the test line and control line capture zone(s). The test line contained target-specific antibody (i.e., a capture agent), which captured, in a sandwich immunoassay format, any gold labelled anti-target antibody-target complexes.

The test cassette was left to sit for 10-30 minutes for the assay to develop. Next, a correct test protocol corresponding to the sample and test panel was selected on the reader system's user-interface (see, e.g., WO2013/014540 for exemplary reader systems). The test cassette was placed in the reader system. A sample identification was entered (via the integrated test entry device or external barcode reader for cassettes with barcodes). The system then carried out a scan of each test strip and calculated assay results (i.e., measurements of one or more targets). Results were displayed on the screen and automatically saved. A standard response curve was generated for each assay. This curve characterizes the response of the assay to a range of concentrations of the associated target in the selected matrix. Curves were produced by assaying multiple replicate samples at specific target concentrations and fitting a mathematical function to the response. With reference to these curves, quantitative measurements of target concentrations may then be estimated on a sample with unknown amounts of each target. Control samples are not run with each test. Rather, there is a QC cartridge that confirms that the machine is operating properly. This cartridge doesn't require any sample addition to run, you simply place it in the machine and run a QC check (˜1 minute). The “Pass” result confirms the machine optics are working properly. Other control steps for assay methods as disclosed herein include that each strip is internally calibrated. Specifically, during manufacture a QC file is created that is specific for a batch of cassettes; a control line controls for fluidics by test line/background intensity; a quality threshold has to be reached for the control line that corresponds to the batch qualification file (this is incorporated in barcode QC batch file); a QC batch file will inform if cassette sat too long after loading and/or quality of sample (based on background).

Tests are also being performed to determine how much time can pass before a strip dries out such that signal will be impacted and alter results.

Exemplary protocols for Comp act detection of sC5b-9 in blood or urine are as follows:

Comp Act sC5b-9 Urine

Materials Needed: —Comp act sC5b-9 Urine; Rapid Test (single use, duplicate, room temp); Steps: 1. Remove duplicate Comp act sC5b-9 Urine Rapid Test and place on flat, level surface.; 2. Pipette 100 μL of neat urine sample into the sample port each cassette (2 total).; 3. Incubate Rapid Test at 30 min.±1 min. at room temp (15° C. to 25° C.); 4. Record Kit ID (Lot #expire, location) in CRF.; 5. End of 30 min., Reader previously turned ON, select sC5b-9 Urine Quant TEST on Comp act Reader. Scan Subject barcode.; 6. Place cassette into Reader drawer, close test cassette drawer and read. Record test result Pass/Fail.; 7. If Test result “Fail” rerun the cassette.; 8. Repeat steps 5-6 with duplicate cassette.

Comp Act sC5b-9 Blood

Materials Needed: —Test Device (single use, duplicate, room temp); —Sample diluent Tube (refrigerate until single use); Steps: 1. Allow Sample Diluent to equilibrate to Room Temp.; 2. With Subject's blood sample, pipette 20 μl of blood into the diluent tube. Close lid and mix thoroughly creating a 1:20 dilution.; 4. Remove duplicate Comp act Rapid test, place on flat level surface.; 5. Pipette 100 μL of diluted sample into the sample port for each cassette.; 6. Incubate Rapid Test at 30 min.±1 min. at room temp.; 7. Record Kit ID (Lot #, expire, location) in CRF.; 8. End of 30 min., Reader previously turned ON, select sC5b-9 Test on Comp act Reader. Scan Subject barcode.; 9. Place cassette into the Reader drawer, close test cassette drawer and read. Record test result Pass/Fail.; 10. If Test result “Fail” rerun the cassette.; 11. Repeat steps 8-9 with duplicate cassette.

When running assays as disclosed herein, certain warnings, cautions, and/or limitations should be kept in mind. In particular, the following should be taken into account:
Perform Duplicate Tests at room temperature (15° C. to 25° C.).
Standard precautions for handling infectious agents should be observed.
Wear standard personal protective equipment clothing when handling specimens and assay reagents in accordance with local regulations.
One sample per cassette. Each cassette is single use and cannot be re-used. Once cassette read, it can be disposed in clinical waste.
A cassette must be used within 30 minutes after removing from foil.
Do not adjust the volume of the sample or the pre-aliquoted samples.
Any variation in the procedure may invalidate the results.

Samples may be detected using reader systems such as Comp act or RapiPlex detectors (see, e.g., U.S. Pat. No. 9,199,232 for description of RapiPlex cartridges with six channels).

### Example 3: Proof-of-Concept for Loss of Signal Titration in Presence of an Abundant Target

The present Example provides a method that measures a level of one or more targets in a sample, over a broad, dynamic concentration range, without having to dilute the sample prior to measurement. The present Example overcomes several shortcomings of prior methods with a combination of approaches as described herein. This Example describes innovative methods of analyzing and quantifying one or more targets in a fluid as compared to other methods of analysis.

As proof-of-concept for the methods developed in this Example, plasma-purified C5 was spiked into sample buffer and compared to a sample that included C5 and a C5 binding agent (anti-C5 antibody). As can be seen in FIG. 7A, in the sample buffer with C5 (left panel), titration of a detectable signal across the measured range of 0-10 μg/mL was not achieved as the test line peak signal intensity did not vary with varying C5 concentration, demonstrating a challenge associated with assays of high abundance targets. In the sample buffer with C5 and anti-C5 antibody, test line peak signal intensity did vary more with different concentrations of C5, demonstrating that without a binding agent of free C5, no differences in concentrations were detectable, despite concentration having been clearly controlled at the outset, thus concentration dependent titration was still not achieved (right panel). These results demonstrate a challenge in assays with high abundance targets specifically showing that a high level of C5 can prevent titration of a signal across a particular range of concentration measurements, which can present a challenge when trying to determine clinically relevant values of a target. Technologies described herein help to overcome this challenge and provide assays that can detect targets that may exist at a high concentration in a given sample, thus providing a reliable, sensitive, accurate, reproducible, reliable, rapid and specific method by which to measure a target. As FIGS. 7B and 7C, demonstrate multi-line approaches were utilized to overcome saturation caused by a high abundance target as seen by concentration-dependent titration of target levels, ultimately resulting in accurate measurement of free versus therapeutic-bound C5, respectively and successfully overcoming challenges shown in FIG. 7A.

As compared to previous assays where the limit of detection was 3.1 ng/ml (e.g., Hycult assay with unknown lower limit of quantitation and a range of 3.1-200 ng/ml) the present assay has a LLOQ of 0.001 μg/mL with a range of 0.001-100 μg/mL.

### Example 4: Modified Lateral Flow Assays to Detect Abundant Targets in Undiluted Samples

To overcome challenges based on inability to detect differences in concentration of an abundant target, updated methods of detection were developed. Here, a lateral flow assay was modified to improve upon various aspects and develop assays that are able to accurately detect and measure an abundant target in a sample.

### Multi-Line Detector Elements on Solid Supports Improve Dynamic Test Range and Eliminate Need to Dilute Samples

First, an assay using multiple detector lines was developed. In this assay, multiple detector lines were placed (“striped”) on a solid phase over which the sample flowed. These detector lines provide a way to absorb “excess” target in a sample (that is, an amount of target that is so great that it distorts results of the assay). By absorbing a quantity of target in at least a first detector line, subsequent detector lines can be used to accurately and reliably detect and quantify a target. FIG. 3 shows the multiple test line configuration of an exemplary test strip as used in this assay.

The effectiveness of multiple test lines, such as depicted in FIG. 3, was demonstrated by spiking C3 into sample buffer and then performing a 7-fold serial dilution. The concentration of C3 in each dilution was then assessed using multiline, multi-detector LFA test strips.

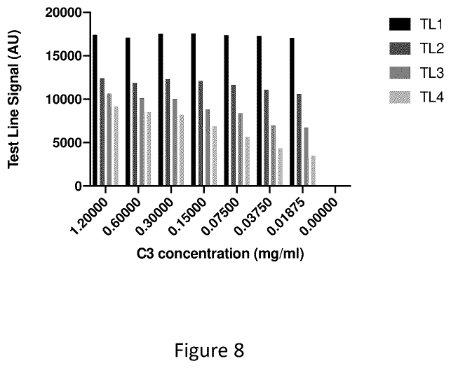

As shown in FIG. 8, test line one is saturated in all samples (i.e., “reads” as if the same concentration of C3 is present in all samples). Test line two is slightly less saturated, but does not clearly distinguish between different concentrations of C3. However, lines 3 and 4 show that the test line intensity signal decreases in presence of decreasing C3 concentrations. This demonstrates that an accurate and sensitive quantitative assay can be performed by using multiple test lines as a way to remove “Excess” target to allow accurate measurement of a concentration in a sample, without having to dilute the sample before measuring the target.

### Increased Surface Area of Solid Supports Improves Accuracy and Dynamic Test Range and Eliminates Need to Dilute Samples

As an alternative or in addition to multiline LFAs, as shown in FIGS. 6A and 6B another approach used to accurately measure “high concentration” targets (without having to dilute a sample) was to improve sensitivity of test strips in the LFA, but in a way that did not impede flow or function of the LFA. Two types of detector beads were compared in this assay: 60 nm gold beads and 400 nm polystyrene beads. Samples spiked with decreasing concentrations of C5 were tested on LFAs that had multiple lines and either 60 nm gold or 400 nm polystyrene beads. LFAs with larger beads were able to better distinguish between differences in high abundance target concentration than those with smaller beads (see FIG. 6B). The larger beads improved the range over which a target can be accurately and quickly measured without impairing the speed or detectability of the target. The combination of increasing surface area of beads and a multi-test line approach improved measurement of a high concentration target to a much greater degree than would have been expected and increased the dynamic range of detection of the target. Similar assays were conducted with C3, with similar outcomes (data not shown).

### Reduction of Prozone Effect by Addition of Competing Agents

During assay development, a prozone effect was sometimes observed. To reduce prozone effect, a competing agent was introduced into the assay system to bind high concentration targets in undiluted samples. This approach was tested in two ways: (i) addition of free antibody to the conjugate pad; and (ii) addition of free antibody to detector before striping.

### (i) Free Antibody on Conjugate Pad

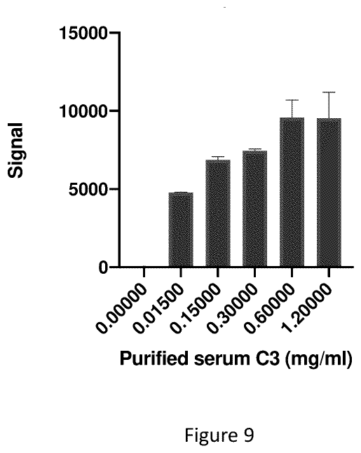

Unlabeled C3 antibody was added to the conjugate pad at 35 nanograms and samples of purified serum C3 in assay diluent were then added. As can be seen in results shown in FIG. 9, addition of competing (i.e., unconjugated, unlabeled, unbound to a support, etc.) agent to the conjugate pad is able to improve accuracy of the assay and detect and quantify amounts of C3 with more sensitivity over a greater range.

### (ii) Competing Agent Added to Lines

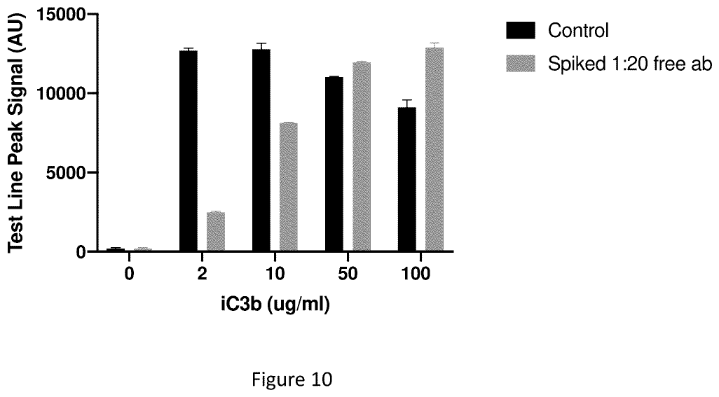

In this assay, competing agent (e.g., unlabeled antibody at a 1:20 final dilution) was mixed in with the diluent prior to adding the diluted sample to the LFA. This effectively transforms a competing agent into a capture agent, but is used for purposes of diluting a portion of a target from a sample, rather than for measuring. Competing agent was also added to the conjugate pad. As shown in FIG. 10, addition of capture agent to multiple test lines improved the accuracy and sensitivity of iC3b measurement in combination with competing agent included in the diluent as compared to measurements without competing agent.

Accordingly, as shown herein, addition of competing agent to the conjugate pad and/or test lines (“stripes”) on a test strip can reduce or eliminate prozone effects and improve accuracy and range of detection of a target in an assay.

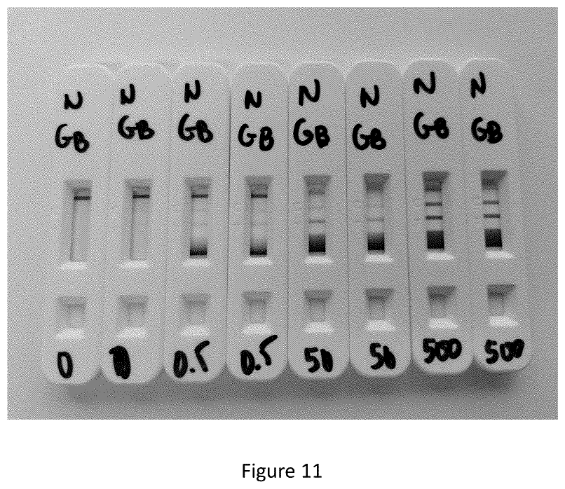

Finally, it was observed that in some assays, sample was getting stuck or not flowing onto the membrane/into the cassette as expected. Upon further investigation, it was discovered that the sample was actually getting “clogged” on the test strips and concentration measurements were inaccurate. Replacing the NC membrane with a higher flow rate solid support increased the rate at which the sample entered and flowed through the test strips and accuracy was improved. Overall, as shown in FIG. 11, changing the membrane and, thus, the flow rate of the sample, reduced test line concentration by approximately 30%.

Thus, as described in the present example, each feature, alone or in combination with one or more other features, provide assays that can measure high concentration targets in a sample without having to dilute the sample prior to measurement, which is something that, until now, no single assay could achieve.

### Example 5: Measurement of Free C3

The present Example describes assessment of an exemplary target, C3. To begin, donor plasma was spiked with increasing concentrations of an exemplary C3 therapeutic drug (C3 binding agent). As shown in FIG. 12, plasma was spiked with increasing concentrations of C3 binding agent, incubated at 37° C. for 60 minutes, and then measured on the Comp act system using a C3 LFA rapid test, which was manufactured in accordance with Example 1 of the present disclosure.

As shown in FIG. 12, increased concentrations of C3 binding agent correspond to decreased concentrations of free C3. That is, increasing concentration of a therapeutic drug decreases concentration of free target (C3) in the plasma sample. Furthermore, off-line dilutions of samples can create artifactual levels caused by dissociation of endogenous C3 from C3-binding agents. Since technologies of the present disclosure provides technologies that overcome these challenges, results from assays described and exemplified herein can be used by clinicians to impact treatment of patients. The approaches described herein stabilize binding agent: C3 interaction and allow for accurate measurement of “free” C3 vs binding-agent associated C3 (e.g., “drug-bound” C3, when the C3 binding agent is a C3-binding therapeutic).

The approach used with measurement of C3 can be translated and applied to other proteins within the complement system, as well as cytokines and other biomarkers present in samples that can be analyzed without dilution (e.g., blood, plasma, urine, etc.)

### Example 6: Measurement of sC5b-9 in Urine

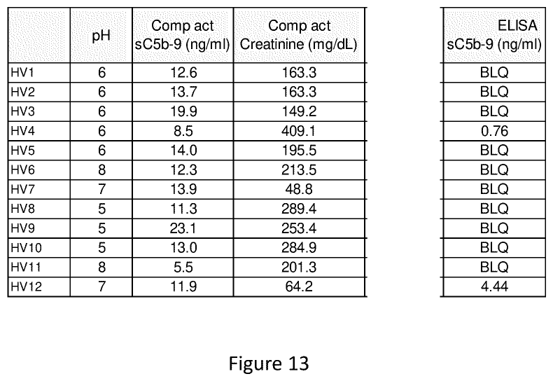

Measuring certain targets, particularly in certain types of samples, can be problematic. For example, measuring sC5b-9 in urine can be very useful and an important predictor and/or indicator of disease, disorder or condition activity or risk of disease, disorder or condition. However, measuring sC5b-9 in urine has generally been problematic for reasons such as low target levels (relative to blood) that are not accurately measured by current methods such as ELISA (FIG. 13) due to a lower limit of quantitation/detection being too high to detect certain levels of target in certain samples, even at the theoretical LLOQ, and such assays are also optimized for blood and/or plasma, and not urine (see, e.g., Quidel ELISA with LLOQ of 8.8 ng/ml; published sandwich ELISA, 2.2 ng/mL using a Dako antibody; BD C5b9 ELISA, range of 0.469 ng/ml to 15 ng/mL, etc.). Furthermore, methods to normalize urine volumes are needed.

To overcome this challenge, creatinine measurements were added to assays to normalize urine concentration. In addition, such traditional types of assays do not facilitate turnaround times needed for point-of-care analysis, which is an important feature for tests monitoring many targets including sC5b-9. Finally, given that a level of a target in urine may be low (e.g., as compared to in blood, plasma, etc.), having an assay that does not require one or more wash steps (such as required in an ELISA) is important to minimizing any loss of sample, target, etc.

Problematically, traditional tests (e.g., ELISAs) for measuring one or more targets in blood or plasma do not perform accurately in urine due to sample matrices in those tests; furthermore, differences in urine volume (and, e.g., target concentration) as compared to blood or plasma volume used in these assays requires normalization for interpretation. Even in blood, however, testing can require significant offline dilutions or several serial dilutions in order to obtain measurements within a test range of 100-100,000 ng/mL. In contrast, technologies disclosed herein have developed assays with improved sensitivity and specificity, including a lower limit of quantification for sC5b9 at 1 ng/mL and simultaneously maintaining a measurement range of 1-100,000 ng/mL.

The present Example overcomes these challenges by developing an assay that can accurately measure one or more targets in a sample of undiluted urine. For example, in urine, sC5b-9 may be present between 5 ng/mL and 200 μg/mL. That is, as will be understood to those in the art, in “disease” states, sC5b-9 may be detected at concentrations of 200 μg/mL, whereas in absence of disease states that impact sC5b-9 levels, concentrations may be well under 250 ng/mL, with as low as 5-30 ng/ml.

In this assay, limits of detection of sC5b-9 were expanded. Specifically, on previously available assays, sC5b-9 is often underreported, either in presence or concentration. This assay recognized that sources of problems in measuring sC5b-9 in previously available assays included narrow limits of quantitation, pH that was not compatible between different samples (e.g., blood and urine), and differences in sample volume and concentration. In particular differences in sample volume and concentration were problematic with urine samples, which can cause differences in concentrations of one or more targets that were not accounted for in measurements of sC5b-9 on previously available assays. The present Example not only recognized sources of such problems, but developed solutions to those problems.

Accordingly, in some versions of assays described herein, for measuring targets in urine, a pad was added between detector and NC to allow mixing/diffusion of sample/detector before reaching NC for urine-based assays (see, e.g., Tsai, et al. Scientific Reports, (2018) 8:17319, and Lu, et al., PNAS, Dec. 19, 2017; 114 (51); 13513-13518; each of which is incorporated by reference herein in its entirety). Here, an assay was developed for detection of a target in an undiluted urine sample. Sample buffer was modified by determining and optimizing pH, which stabilized urine samples and allowed for measurements in a more efficient, accurate, specific, reliable, sensitive and/or rapid way than in previously available assays. That is, pH of the sample was stabilized/optimized in the sample pad.

In sC5b-9 assays comprising a sample that does not include urine, buffer chemistry may differ; that is, in a given cassette, buffer chemistry depends on assays and antibodies/matrices being used such that buffer chemistry for urine is different from that of buffer chemistry in an assay measuring one or more targets in blood. For example, when whole blood samples were applied, a filter for red blood cells was used wherein the filter has a mechanism (e.g., an anti-RBC antibody) to capture red blood cells. In samples of urine, the sample pad was optimized to buffer over a range of urine pH levels.

On multiplexed assays (e.g., performed using a RapiPlex reader; see, e.g., WO 2013/014540, which is herein incorporated by reference in its entirety), buffer chemistry may be changed between channels which allows optimization of each channel and multiplexing of tests that were previously or are otherwise incompatible with one another. Furthermore, this assay was improved by doubling detector concentration and adding multiple lines on the test strips (see FIG. 2B showing results from a test cassette with a test strip comprising four test lines). The increase in detector concentration provided significant improvement in sensitivity of target detection and quantitation; however, by increasing sensitivity in target detection, lower limit of quantitation was improved, but upper limit of quantitation was impaired (i.e., the LLOQ was improved, but the range was negatively impacted due to saturation). Detector concentration per test was not found to be a limiting factor in the assay. Indeed, increasing detector concentration increased background signal and masked test line signals, which negatively impacted sensitivity. In order to be able to detect lower-level concentrations without dilution as well as upper levels (i.e., closer to ULOQ), additional test lines were added onto test strips that previously only contained a single line (see, e.g., FIG. 2B). Thus, sC5b-9 range detection in urine samples was expanded. As compared to previously available assays, samples that were reported as not having any sC5b-9 on previously available assays showed positive results for measurement (i.e., detection and quantification) on assays of the present disclosure (see, e.g., FIG. 2B).

Finally, in addition to an sC5b-9 assay, a creatinine assay was also run using each sample; creatinine also serves as an important normalizer of urine volume in the LFA. This assay is also advantageous in that as it does not require any wash steps, there was no loss of material during testing as compared to traditional ELISA tests, such as the commercially available test shown in FIG. 13. This approach allows measurement of low levels of sC5b-9 (e.g., 5-30 ng/mL), which have, until now, been undetectable in previously developed or currently marketed assays.

Here, urine samples from 12 healthy volunteers were evaluated for sC5b-9 on the LFA assay developed herein and run on the Comp act system; these results were compared to those from a standard, commercially available ELISA kit. As shown in FIG. 13, sC5b-9 was detected in urine of all 12 samples using the LFA assay developed herein, whereas only 2 of samples showed detectable sC5b-9 in the urine using a commercially-available ELISA kit.

As also shown in FIG. 13, sC5b-9 and creatinine were measured in the same sample using the Comp act system; this is not possible using an undiluted sample in an ELISA assay, which can only measure one particular target per well and portion of sample. Assays describe in this example show that range for detection of sC5b-9 in urine has been improved to 0.5 ng/ml to 10 μg/mL as compared to, e.g., commercial assays, e.g., Quidel, with a range of 8.8 ng/ml-190 ng/ml. As described herein, there is no prozone effect with assays of the present disclosure at concentrations up to 200 μg/mL). In addition to such a vast improvement in range of detection, detection of two distinct targets (e.g., sC5b-9 and creatinine), within the same sample also supports the development of multiplexing of two or more targets on a RapiPlex system. Ability to use an undiluted sample allows detection of a broad range of sC5b-9 and increases utility in clinical settings where patients may have a variety of different conditions or levels of one or more targets; this improved range of sC5b-9 along with improved range and ability to detect and quantify other targets from the same sample, without need for additional steps such as dilution improves efficiency (and speed of results), and also reduces risks of error or artifact (from, e.g., dilutions) that may exist in other systems that require, e.g., dilutions or multiple sample inputs.

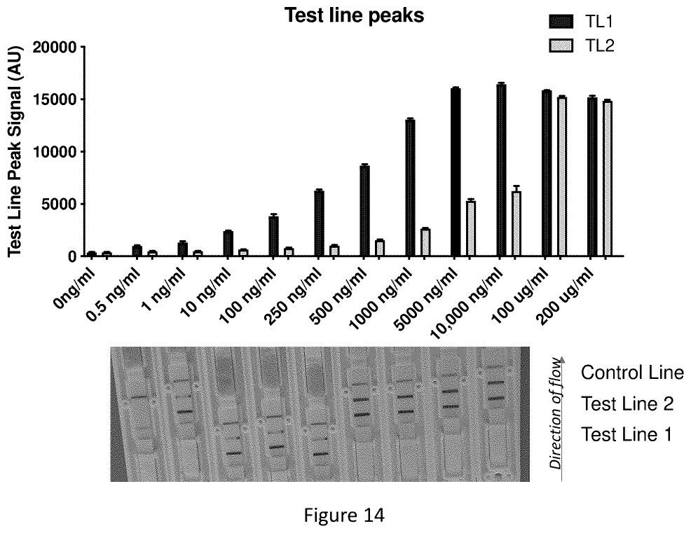

Breadth of test range was also determined and improved using laboratory-prepared samples; assembled cassettes were tested with sC5b-9 Buffer QC Panel (P/N 3010) and high concentrations prepared from C5b-9 antigen (P/N 1165) in TBS Casein 0.1% T20 (P/N 2066). As shown in FIG. 14, addition of multiple lines onto test strips further improved test range capabilities. Samples were quantified from 0 ng/ml up to 200 μg/mL.

Combined, increased detector concentration and multiple test lines provide an assay with a dynamic test range between 0.5 ng/ml-100 μg/mL and an upper range of quantitation of 10 μg/mL-100 μg/mL. It was also determined that at concentrations of up to 200 μg/mL no prozone effect was observed. These improvements are paramount to function of the assay with an undiluted sample and show that undiluted urine is a viable indicator of important targets that can be used to monitor clinical status of subjects in situations such as therapeutic monitoring in real-time (e.g., during treatment, during clinical trials, etc.). That is, importantly, as described herein, the present Example describes creation of assays with high negative predictive values, thereby improving problems associated with false negatives in previously available assays. Such improvements will increase likelihood that a patient in need of treatment will receive timely and accurate treatment rather than not receiving treatment due to an inaccurate (e.g., false negative) target measurement.

### Example 7: Improved Measurement of ADAMTS13

The present Example describes ability to rapidly, specifically, sensitively, reliably, and accurately measure ADAMTS13 in a sample. ADAMTS13 is an enzyme that cleaves VWF, which is involved in blood clotting. Importantly, deficiencies in ADAMTS13 activity can cause decrease or loss of cleavage ability, resulting in deficiencies in blood clotting. Such deficiencies may be inherited or acquired; regardless of etiology of deficiencies, timely and accurate diagnosis is paramount to receiving proper (and often lifesaving) treatment. For example, a decrease in activity of ADAMTS13 may indicate that a sample is from a patient with TTP; a treatment for TTP is plasma exchange and, if delayed, fatality can result. However, TTP can also have similar symptoms as aHUS, and if a patient with aHUS has plasma exchange due to inaccurate diagnoses, it can actually be harmful to the patient and also cause delay in receiving proper treatment. Accordingly, as described herein, an assay that can reliably, accurately, specifically, rapidly, and sensitively measure ADAMTS13 activity provides a solution to an unmet need in the field.

ADAMTS13 activity can be measured in any sample comprising VWF (e.g., any genotype, e.g., plasma or whole blood), as the assay measures activity of ADAMTS13 on cleavage of VWF. Here, cleavage of a recombinant VWF was measured (using the Comp act system and reader system). This rVWF fragment was 175 amino acids in length and includes a six-histidine tag at the C terminus, for use with ADAMTS13-mediated cleavage studies. The C-terminal cleavage fragment was measured in the sample, after the sample was combined with the rVWF substrate; the capture agents and detecting agents (anti-C-terminal antibody and anti-his tag antibody) were added to the immunoassay device and comprised a labeled antibody that specifically recognized the C-terminal cleavage fragment, but not uncleaved VWF, nor does it capture or recognize the N-terminal cleavage fragment and also comprise an antibody that recognized the 6 his tag on the C-terminal fragment. In this Example, the 6 his tag antibody was the detecting agent and the C-terminal antibody the capture agent, but competing/capture and detecting agent binding partners can be reversed. The N-terminal fragment was not and is not measured; it passes through the solid phases of the assay and is not captured, nor does it react with any detecting and/or competing agents.

To measure of ADAMTS13, 50 μl of sodium citrate plasma was added to a tube labelled “VWF Cleavage reaction” (which tube contained 2 μg/mL recombinant VWF in CaCl2 buffer (50 μl volume)). This was then mixed with a pipette and incubated for 10 minutes at 37° C. Any ADAMTS-13 in the sample (dependent on the activity level (ranging <10%-100%)) cleaved the rVWF. After incubation, 2 μl of “Termination Solution” (containing 0.5M EDTA) was added to the cleavage assay reaction tube to terminate the cleavage reaction. EDTA has a chelating effect on the ADAMTS-13 enzyme's activity and thus halted any further cleavage during the next steps. 30 μl of solution from the VWF Cleavage Reaction tube was added to the ADAMTS-13 Sample Diluent tube and mixed well by pipetting (1 in 10 dilution). 100 μl of solution from the ADAMTS-13 Sample Diluent tube was pipetted onto the sample port of the ADAMTS-13 Activity cassette and incubated for 30 minutes #1 minute at room temperature. A polyclonal goat Ab for cleaved/intact VWF was used as the capture agent (i.e., to capture the C-terminal cleavage fragment) and a murine monoclonal Ab specific for cleaved VWF neoepitope was used as the detecting agent. Internal controls for ADAMTS13 were added at different ranges to develop appropriate concentrations for competing, capture, and detecting agents. That is, recombinant ADAMTS13 was used to establish range of activity for this assay, which also provided data to establish high and low control conditions/ranges dependent upon amount of recombinant ADAMTS13 added into the system.

Initial assay results showed cross-reactive antibodies between C-terminal fragment and uncleaved VWF, but further development was conducted, and highly specific assays were developed. Data collected in a finalized version of this assay (not shown) indicate that the measurement using the C-terminal-specific antibody successfully recognized only cleaved VWF. These data are obtained in a much more rapid and direct fashion than any previously available assays. That is, the assay in this Example takes a maximum of approximately 40 minutes, whereas a commercially-available ELISA takes three hours and requires much more handling and sample processing, and a commercially-available fluorometric assay requires one hour and additional handling and measurement steps; neither assay is available as a point-of-care test. Here, the only additional step is a pre-processing incubation of a sample with the rVWF substrate which takes between 5-10 minutes, prior to applying the sample to a test cassette of the present disclosure for measuring ADAMTS13 activity levels. Multiplexing of this ADAMTS13 assay is also, therefore, not problematic because the sample dilution is approximately 1:10-1:20 (i.e., a 1:1 cleavage assay step followed by 1:10 dilution step with buffer was performed in this Example; furthermore, buffers used for samples comprising blood and plasma are compatible with a sample that has been combined with the rVWF substrate, and, therefore, once the rVWF incubated substrate is applied to a test cassette of the present disclosure, one or more additional targets (other than cleaved rVWF as surrogate for ADAMTS13 activity) can be measured in accordance with contents and capabilities of the particular test cassette used.

In assays according to the present Example, it is expected that samples from subjects without a disease, disorder, or condition that impacts ADAMTS13 activity would give a “strong” test line due to high VWF cleavage; that is, at least 50-68% activity. In contrast, subjects with TTP would be expected to have approximately less than 10% ADAMTS-13 protein or activity as compared to subjects without TTP, and, accordingly, would induce minimal cleavage and thus, test lines would be “weak” or absent.

In developing this assay, it was important to determine whether endogenous ADAMTS13 (i.e., ADAMTS13 in a sample from a subject) was capable to cleave rVWF. Several optimization experiments were conducted to verify whether plasma samples needed additional conditions (e.g., pH, salt, temperature) for successful and accurate cleavage activity. To confirm, tests of healthy plasma (e.g., from a subject not known to have a disease, disorder or condition that would impact ADAMTS13 activity) was incubated with rVWF at increasing plasma concentrations and it was found that VWF cleavage increased proportionally in response to concentration of endogenous ADAMTS13 in plasma.

Antibody characterization was performed for 6-His tag antibodies and rVWF antibodies to confirm which antibodies would be able to accurately quantify ADAMTS13 activity and distinguish between cleaved and uncleaved rVWF. Additional conditions such as pH and chelating agent (e.g., EDTA, sodium citrate) were tested and compared to determine optimal assay parameters for measuring ADAMTS13 activity. Accordingly, the present example provides a proof-of-concept for successfully measuring ADAMTS13 by detecting cleaved rVWF on an assay using both spike plasma samples or samples from subjects, wherein the assay provides results in no greater than 40 minutes and with minimal pre-processing steps from sample collection to target measurement.

ADAMTS13 measurement is reported as a percentage of “normal.” In a sample from a subject suspected of having TTP, if ADAMTS13 activity is measured at <10%, TTP diagnosis is confirmed. In a sample from a subject, if ADAMTS13 activity is between 10-30% a patient is at risk or susceptible to having or developing TTP; in such a situation, additional testing would be indicated to investigate differential diagnoses and/or whether any inhibitor such as an autoantibody to ADAMTS13 is present or other conditions or factors are contributing to measured ADAMTS13 activity level If a sample from a subject has ADAMTS13 activity >30% (and is clinically suspected of having aHUS or TTP, the patient is diagnosed as having aHUS).

If a patient is at risk of having, susceptible to or at risk of complement-mediated TMA, ADAMTS13 activity should be measured; if ADAMTS13 activity is <10%, then the patient is suspected as having or diagnosed as having TTP; if ADAMTS13 activity is 10-20%, the patient is suspected as having TTP, but in context and as will be understood by one of skill in the art, additional clinical judgement or further testing and/or monitoring should be undertaken in patients with ADAMTS13 activity levels of about 10-20% (see, e.g., guidelines for TTP diagnosis at Zheng et al., J Throm Haemost. 2020; 18:2486-95, which is herein incorporated by reference in its entirety). If a patient is diagnosed as having TTP, treatment comprises plasma exchange. If a patient is diagnosed as having aHUS, treatment does not comprise plasma exchange.

### Example 8: Measurement of Low Abundance Targets

The present Example describes assessment of an exemplary cytokine, IL-6. To begin, 19 undiluted donor plasma samples (previously collected and stored at −80 degrees Celsius) from patients with RA, lupus, or psoriasis were measured on the RapiPlex system in accordance with Example 1 of the present disclosure.

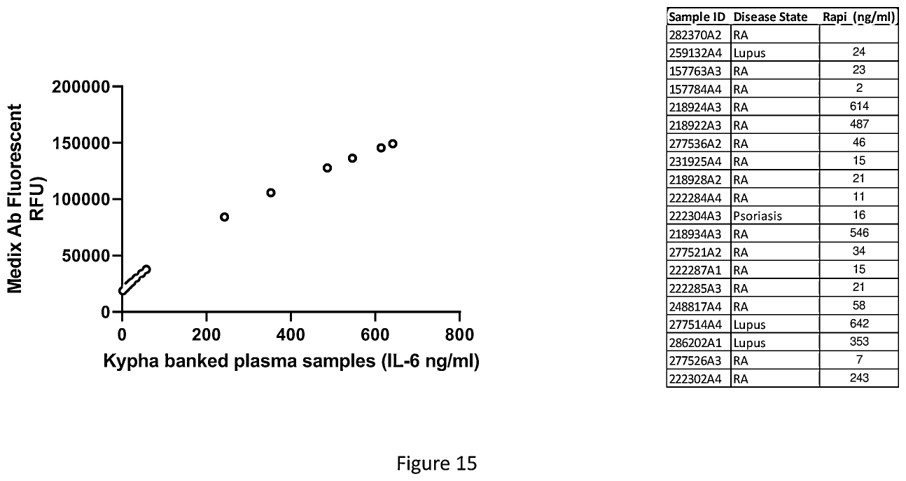

Concentrations as low as picogram quantities of IL-6 can be detected using strips and cassettes manufactured in accordance with Example 1 herein. A “normal” reference range for plasma IL-6 is considered less than or equal to 1.8 pg/mL. In conditions such as septic shock, levels up to 11,062 pg/mL have been reported (with a median of 1378.6 pg/mL) and in sepsis, a median of 89.9 pg/mL. Since the RapiPlex system has six channels and each channel can accommodate up to four distinct tests, it is possible to separate “low” and “high” abundance targets to ensure accurate measurements. Furthermore, each channel can be optimized so that all tests within a channel are working optimally and without interfering with any of the other channels. As shown in FIG. 15, IL-6 was detected in 18 of 19 samples tested, at levels in a range of 0-800 ng/mL. Since this test was performed on the RapiPlex platform, it also shows that measurement of low concentration (e.g., pg or ng/ml) cytokines (e.g., IL-6) can be combined with multiple other targets, such as high concentration (e.g., mg/mL, e.g., C3) targets on a single test cassette having multiple test strips each with multiple test lines that can be measured using multiple visualizable channels (e.g., a LFA as described herein) using an undiluted sample such that proteins within the complement system, as well as cytokines and other biomarkers present in samples that can be efficiently, accurately, sensitively, specifically, efficiently, and/or reliably detected and analyzed without dilution (e.g., blood, plasma, urine, etc.)

This provides multiple readouts from a single, small undiluted patient sample, improving overall efficiency, speed, accuracy, sensitivity, specificity, and reliability for unrivaled patient monitoring in a variety of settings (e.g., clinic, home, etc.) Presence and concentration of IL-6 was determined in samples undiluted plasma within 30 minutes of reconstitution.

### Example 9: Multiplexing High and Low Abundance Targets

The present Example describes assessment of an exemplary targets that typically occur with concentrations that differ on order(s) or magnitude. As described herein, the technologies provided by the present disclosure allow complexing of targets that traditionally would have required different assays. For example, devices of the present disclosure can multiplex up to 24 targets in a single sample/single test cassette with up to six assay channels read on a single scan (i.e., on the RapiPlex reader). Thus, this system allows up to four targets with similar assay chemistry and/or similar working/detection ranges of targets to be complexed in each of the 6 channels, eliminating a need for, e.g., multiple tests on e.g., multiple aliquots from a single sample or, e.g., more than one test of a single target within a single sample to ensure an accurate measurement). Such a system increases efficiency accuracy, sensitivity, specificity, reliability and/or speed of detection of multiple targets from a single undiluted sample (or portion thereof). Furthermore, measuring all targets simultaneously provides levels of multiple targets in a sample without complicating influences of, for example, time-dependent changes, assay reagents/materials or cross-talk (e.g., between reagents used to measure, e.g., detecting agents, e.g., competing agents, e.g., buffers, etc.) impacting concentrations (actual or detected) of targets if, for example, measurements were performed at separate time points and/or in separate assays from a single sample.

In this example multiplexing of targets that are typically high and low abundance targets (e.g., those detected on mg and pg quantities, respectively) are measured using a single sample and single cassette with multiple test lines on a test paper. Here, an undiluted sample is tested for presence and quantity of C3 and IL-6 using an LFA rapid test comprising a test strip with test lines capable of detecting C3 and IL-6, which was manufactured in accordance with the present disclosure (see, e.g., Example 1).

Presence and concentrations of C3 and IL-6 are determined in an undiluted sample (e.g., plasma and/or blood) within 30 minutes.

### Example 10: Multiple Lines for Improved Target Specificity

The present Example describes overcoming lack of antibody specificity to specifically and accurately detect and/or quantify a target. As described herein, detection of certain targets can be inaccurate and/or non-specific due to lack of antibody specificity.

Using an approach as described herein (e.g., see Example 1) cassettes with multiple lines are created and targets with known antibody specificity challenges (e.g., Factor B, C3, iC3b, etc.) are used in order to remove cross-reacting proteins using first lines which capture cross-reacting proteins but do not detect them. After capture is performed, the next line(s) are used to detect and/or quantify one or more targets.

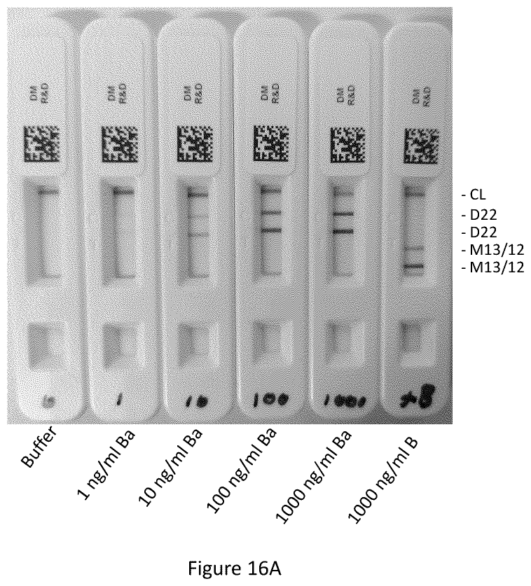

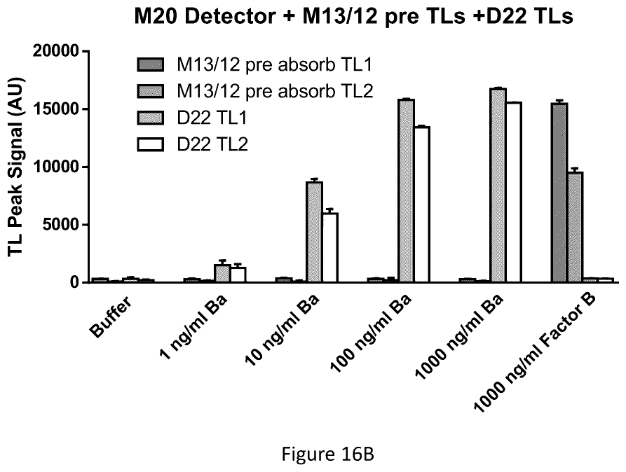

Here, in one use of a multiple-test line approach and as a proof-of-concept demonstrated by FIG. 16A, Factor Ba is analyzed by first capturing parent Factor B protein in the first test line(s) of a cassette using an anti-Factor B/Bb antibody (M13/12) that cross reacts with full length Factor B. This removes full-length Factor B, which comprises the Ba fragment and thus cross reacts with many Ba antibodies, causing inaccurate measurement of cleaved Ba (i.e., the target of this assay). The next test line(s) are striped to include anti-Ba antibody (D22/3) as a capture component, which allows for specific and accurate detection of cleaved Ba and is not contaminated by full-length/parent Factor B protein.

In order to assess capture and detection specificity, sample buffer was either run alone or spiked with increasing concentrations of purified Factor Ba (1-1000 ng/ml) or 1000 ng/ml Factor B. The individual samples were applied to the sample port and the assay was allowed a 30-minute incubation period to develop prior to placement of cassette into the Comp act reader system. A dose dependent increase in detection of Factor Ba is shown on the cassette (see FIG. 16A) and quantified (see FIG. 16B).

This approach can be extended to any complement protein with cleavage products and antibodies available to detect whole, fragmented, and/or cleaved complement components.

### Example 11: Multiplexing-Assessment of iC3b, C3 and C4

The present Example describes assessment of exemplary targets that typically require one or more initial lines to modify an undiluted sample for accurate measurement. As described herein, the technologies provided by the present disclosure allow complexing of targets that traditionally would have required different assays and manipulations, but instead use a single undiluted sample and in situ modification of a sample, without sample dilution, to efficiently, accurately, sensitively, specifically, reliably and/or rapidly detect and/or quantify multiple targets. As described herein, devices of the present disclosure can multiplex up to 24 targets in a single sample/single test cassette with up to six assay channels, each with four targets, read on a single scan (i.e., on the RapiPlex reader). In a case where not all channels will be utilized in a given test, redundant testing (e.g., same target on same sample) can also be performed to improve reproducibility, accuracy, etc. (e.g., achieve CV<5%).

In this example multiplexing of targets are measured using a single sample and single cassette with multiple test lines on a test strip. Here, an undiluted sample is tested for presence and quantity of iC3b, C3 and C4 using an LFA rapid test containing a test strip with test lines capable of detecting each target (e.g., on the RapiPlex system), which was manufactured in accordance with the present disclosure (see, e.g., Example 1).

Presence and concentrations of iC3b, C3, and C4 are determined in samples of within 30 minutes of assay start time and as soon as possible, but not to exceed 5 hours post-collection (with sample stored on ice), unless a sample has been previously collected and/or processed and/or frozen. C3 and C4 measurements generally require multiple lines for excess target and competing agent absorption and iC3b generally does not.

### Example 12: Multiplexing-Assessment of iC3b, Ba, sC5b-9 and/or Creatinine in Whole Blood and Urine

The present Example describes assessment of an exemplary target that typically requires one or more initial lines to modify an undiluted sample for accurate measurement. As described herein, the technologies provided by the present disclosure allow complexing of targets that traditionally would have required different assays and manipulations, but instead use a single undiluted sample and in situ modification of a sample, without sample dilution, to efficiently, accurately, sensitively, specifically, reliably and/or rapidly quantify multiple targets. As described herein, devices of the present disclosure can multiplex up to 24 targets in a single sample/single test cassette with up to six assay channels read on a single scan (e.g., on a reader device, e.g., the RapiPlex reader system).

In this example multiplexing of targets are measured using a single sample and single cassette with multiple test lines on a test paper. Here, an undiluted sample is tested for presence and quantity of iC3b, Ba, sC5b-9 and/or creatinine using an LFA rapid test containing a test strip with test lines capable of detecting each target (e.g., detected using the RapiPlex system), which was manufactured in accordance with the present disclosure (see, e.g., Example 1).

Presence and concentrations of iC3b, Ba, sC5b-9 and/or creatinine are determined in samples of within 30 minutes of assay start time. The sample reading system (e.g., RapiPlex) distinguishes between creatinine lines and those for blood-based assays. Detecting agent(s) and/or competing agent(s) will be added to the assay as appropriate for each target.

### Example 13: Multiplexing—Assessment of sC5b-9, IL-6, and iC3b in Whole Blood

The present Example describes assessment of exemplary targets sC5b-9, IL-6, and iC3b that typically require different assays and manipulations to allow accurate measurement. As described herein, the technologies of the present disclosure allow for assessment of complement and cytokine targets from a single sample that would previously have required separate samples and assays for analysis. This multiplexing allows for resolution of crosstalk between the two systems that was previously not possible. As described herein, devices of the present disclosure can multiplex up to 24 targets in a single sample/single test cassette with up to six assay channels read on a single scan (e.g., on a reader device, e.g., the RapiPlex reader system).

In this example, blood was collected from healthy volunteers in sodium citrate tubes and processed to plasma. The plasma was then separated into two tubes for comparison; one unstimulated tube and another tube stimulated by the addition of 250 μl of 100 μg/ml heat-aggregated gamma globulin (HAGG). Those skilled in the art understand that HAGG is a potent stimulator of the classical complement pathway. Baseline measurements of sC5b-9, IL-6, and iC3b were conducted on the unstimulated sample using an LFA rapid test containing test lines capable of detecting each target, which was manufactured in accordance with the present disclosure.

Both unstimulated and stimulated samples were subsequently incubated for 1 hour at 37° C. After incubation, 90 μl of Futhan was added to the unstimulated sample to prevent further complement activation, and concentrations of both sets of samples were measured. Both tubes were then further incubated for an additional two hours at 37° C. and measured for the presence and concentration of sC5b-9, IL-6, and iC3b. All samples were applied to the sample port of the multiplex test cassette at the indicated time points (0, 60 minutes, and 180 minutes) and allowed a 30-minute incubation period for the assay to fully develop.

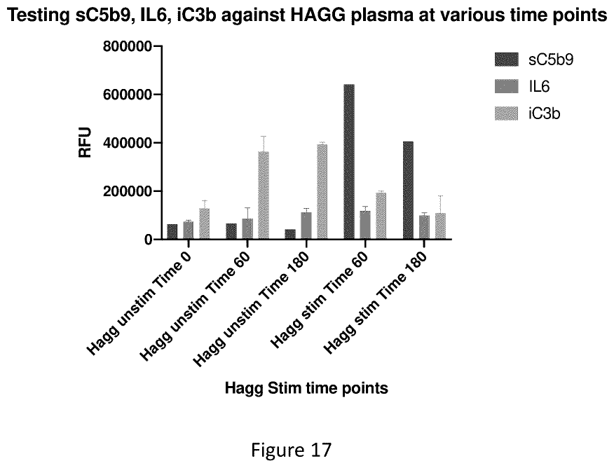

As shown in FIG. 17, the indicated time point measurements of unstimulated (0, 60, and 180 minutes) and stimulated with HAGG samples (60 and 180 minutes) demonstrates the efficiency, accuracy, sensitivity, and reliability to quantify multiple targets from a single sample without individual processing steps being required. As is shown in FIG. 17, iC3b increased after 60 and 180 minutes in the unstimulated sample possibly due to autoactivation. Similarly, HAGG stimulation of the classical complement pathway in the plasma sample resulted in an increase in sC5b-9 concentration, whereas IL-6 underwent no significant changes. Utilization of the assays of the present disclosure allowed for remarkably improved capability for time-course measurements that specifically detect changes in individual analytes on a rapid, multiplex assay.

### Example 14: Diagnosis, Monitoring, and/or Treatment of Complement-Mediated Thrombotic Microangiopathy (CM-TMA), Atypical Hemolytic Uremic Syndrome (aHUS), and/or Thrombotic Thrombocytopeniaurpura (TTP) Using Undiluted Samples

As described herein, technologies of the present disclosure can be used to monitor and/or diagnose immune-mediated diseases, disorders and/or conditions. To diagnose and/or monitor CM-TMA, a cassette is manufactured with lines for detection and quantification of sC5b-9, ADAMTS13 activity, iC3b, C3, C4, C4d, Ba, and/or free C5 (in a sample that also comprises an anti-C5 therapeutic). A sample is applied to the cassette and detected using the RapiPlex system.

If a level of ADAMTS13 activity is determined to be <10% (of a reference sample, e.g., an ADAMTS13 substrate), that sample is more likely to be from a subject with thrombotic thrombocytopeniaurpura (TTP) than from a subject with CM-TMA or aHUS. If levels of sC5b-9 are elevated, aHUS and/or TTP may be present. If levels of iC3b are increased, C3 is decreased, and C4 is at control concentrations, a sample is more likely to be from a subject with atypical hemolytic uremic syndrome (aHUS). Depending upon if a diagnosis of aHUS, TTP, or another observation is made, a subject will be treated with one or more treatments.

### Example 15: Diagnosis and/or Treatment of Hematopoietic Stem Cell Transplant-Associated Thrombotic Microangiopathy (HSCT-TMA) Using Undiluted Samples

As described herein, technologies of the present disclosure can be used to monitor and/or diagnose immune-mediated diseases, disorders and/or conditions. To diagnose and/or monitor HSCT-TMA, a cassette is manufactured with lines for detection and quantification of sC5b-9, ADAMTS13, C3, C4, C4d, Ba, IL-8 and, optionally, IL-6, iC3b, and/or free C5. Without being bound by any particular theory, it is contemplated that in diagnosing and/or treating HSCT-TMA, C4/C4d are of increased importance here (e.g., relative to diagnosis and treatment of aHUS/TTP) because HSCT-TMA is frequently associated with GVHD, which may be detected and/or monitored using, among other things, C4 and C4d levels. A sample from a patient that has undergone an HSC transplant is applied to the cassette and detected using the RapiPlex system and read as compared to a known reference level that is controlled for by the test lot from the cassette lot. Repeat testing is considered useful in this context to understand development of trends and changes in complement proteins over time; changes can occur rapidly, and prompt intervention is important in treating a patient.

If levels of iC3b and/or C4d are increased and C3/C4 are decreased the subject is likely to be experiencing or at risk of experiencing GVHD and/or HSCT-TMA. If, in addition, sC5b-9, Ba, and/or IL-8 are increased, a subject may be more likely to be experiencing or at risk of experiencing HSCT-TMA. If levels of sC5b-9 are within normal ranges, the subject is not as likely to be experiencing or at risk of experiencing HSCT-TMA. Ruling out TTP is important in any TMA since treatments are different and delay in treatment can be fatal. Depending upon if a diagnosis of HSCT-TMA and/or any other abnormality observed in the assay, a subject will be retested and/or treated with one or more treatments.

### Example 16: Diagnosis, Monitoring, and/or Treatment of Lupus Nephritis Using Undiluted Samples

As described herein, technologies of the present disclosure can be used to monitor and/or diagnose immune-mediated diseases, disorders and/or conditions. To diagnose and/or monitor lupus nephritis, a cassette is manufactured with lines for detection and quantification of iC3b, sC5b-9, intact C3 and/or IL-6 and creatinine if a sample is a urine sample or iC3b, sC5b-9, C4, C4d, intact C3, and/or IL-6, and, optionally, free C5 (i.e., if a patient is on an anti-C5 therapeutic), if a sample is a blood and/or plasma sample. As will be understood to those of skill in the art, lupus nephritis is a very heterogeneous disease, so panels with several markers are expected to generate a more accurate and clinically meaningful result as compared to panels for other diseases, where fewer markers may still provide a more reliable and actionable clinical picture. A sample from a patient that has or is suspected to have or develop lupus nephritis is applied to the cassette and detected using a reader system (e.g., RapiPlex system, etc.).

If levels of plasma C4 and/or intact C3 drop in a subject already diagnosed as having lupus is at risk of having a flare; if a subject has not been diagnosed as having lupus, the subject is likely to be experiencing or at risk of experiencing onset and/or a flare of lupus nephritis. If levels of plasma C4d, iC3b and/or sC5b-9 increase, subject is likely to be experiencing or at risk of experiencing onset and/or flare of lupus nephritis. If a subject has been diagnosed with lupus, an increased plasma C4d may distinguish between a patient with lupus nephritis and a patient without renal involvement. If levels of urinary IL-6, iC3b and/or sC5b-9 increase in a subject already diagnosed with lupus nephritis the patient may be experiencing a flare. If levels of urinary IL-6, iC3b and/or sC5b-9 increase in a subject not already diagnosed with lupus nephritis, but diagnosed with lupus without kidney involvement, the subject may have or be at risk of developing lupus nephritis. Depending upon if a diagnosis of lupus nephritis or risk of lupus flare is detected and/or if any other abnormality is observed in the assay, a subject will be treated with one or more treatments. In some instances, treatment may only include monitoring or further testing with or without administration of any therapeutics until and unless additional signs of flare or risk of flare are detected as compared to a first measurement of one or more targets as described in this Example.

### Example 17: Diagnosis, Prevention and/or Treatment of COVID19-Related Cytokine Release Syndrome Using Undiluted Samples

As discussed and described herein, the present disclosure provides new and improved detection, quantitation and multiplexing technologies. Without being bound by any particular theory, COVID19 is a disease that is known to have a vast array of clinical manifestations and outcomes and inflammatory or other biological processes have been, thus far, undetectable in any prophylactic or meaningfully therapeutic way.

Here, a biological sample (e.g., blood, plasma, saliva, urine) from a patient with or suspected of having or having had COVID19 is tested for IL-6, IL-1, C5a, iC3b, CXCL9, sC5b-9, sCD25, Ferritin, and/or MASP2:AT complex on at least one undiluted sample. Depending on which targets are present at which levels, a therapeutic and/or prophylactic intervention may be applied to a patient such that current morbidity or mortalities may be reduced by allowing healthcare providers to intervene in a way or at a time that will alter the clinical manifestation and/or progression of the disease and related sequelae.

### Example 18: Diagnosis, Prevention and/or Treatment of Cytokine Release Syndrome Using Undiluted Samples

As discussed and described herein, the present disclosure provides new and improved detection, quantitation and multiplexing technologies. Without being bound by any particular theory, cytokine release syndrome can occur in response to a vast array of stimuli. Specifically, without being bound to any particular theory, the present disclosure provides methods to diagnose, prevent and/or treat cytokine release syndrome that occurs in response to a disease, disorder, condition and/or treatment of any disease, disorder, and/or condition. In particular, advances in therapeutics (e.g., gene therapy, CAR-T, etc.) provide clinicians with ways to treat previously deadly and/or debilitating diseases, disorders and/or conditions. For example, as is known to those of skill in the art, certain therapies (e.g., CAR-T, gene therapy) can treat certain diseases, disorders and/or conditions in ways that were not possible; however, administration of such therapies includes a risk of adverse events that includes, e.g., cytokine release syndrome. There is currently an unmet need in the field for timely and accurate monitoring of adverse therapy-associated events such as cytokine release syndrome (also known as cytokine storming). If a clinician can monitor, detect and/or intervene before or as a cytokine release syndrome is appearing, a patient may still get a life-saving therapies and an adverse event can be mitigated and/or prevented and interventions can be easily and effectively personalized to improve safety and efficacy of lifesaving treatments in ways that were previously not possible.

Importantly, assays and technologies provided by the present disclosure could serve multiple purposes such as prognostic, predictive, and diagnostic algorithms and information derived from a single cassette and/or monitoring of levels related to therapies or dosages of other therapeutics (e.g., anti-IL6, anti-C3, anti-C5, etc.).

Here, a biological sample (e.g., blood, plasma, saliva, urine) from a patient being treated with a therapeutic (e.g., gene therapy, CAR-T therapy, antibody for chronic illness, e.g., psoriasis, etc.) is tested for IL-6, IL-1, C5a, iC3b, CXCL9, sCD25, Ferritin, sC5b-9, and/or MASP2:AT complex on at least one undiluted sample. Depending on which targets are present at which levels, a therapeutic and/or prophylactic intervention may be applied to a patient such that current morbidity or mortalities may be reduced by allowing healthcare providers to intervene in a way or at a time that will alter the clinical manifestation and/or progression of the disease and related sequelae.

### Example 19: A Method for Segmenting Large Autoimmune Populations

The present Example describes a method to group heterogeneous patient populations based on complement profile. To begin, samples from 18 patients with lupus were collected at a minimum of two time points, one each during a visit with high and low renal disease activity, respectively (with renal activity determined from scores measured using the SLEDAI-2K). sC5b-9 was assessed in the undiluted urine samples in accordance with Example 6 (measurement of sC5b-9 in urine) of the present disclosure.

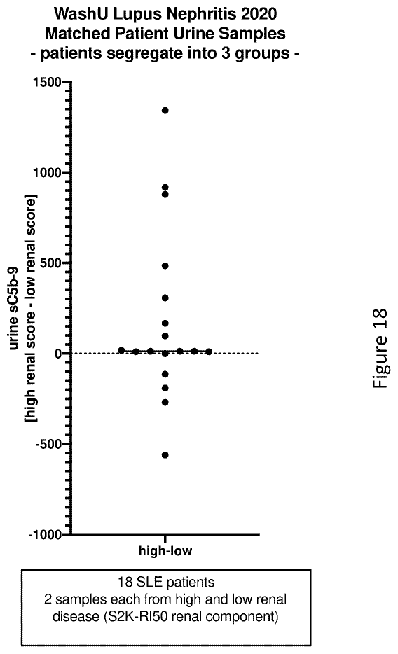

A comparison of sC5b-9 levels in urine from patients between visits in which a high renal score versus a low renal score was recorded (see FIG. 18) These results demonstrate segmentation of patients into three distinct groups: (1) a higher mean sC5b-9 level when renal disease activity was high than when renal disease activity was low; (2) minimal or no change in sC5b-9 levels regardless of disease activity; and (3) low sC5b-9 levels when renal disease activity was high and high sC5b-9 levels when renal disease activity was low. This previously undeveloped method can be used to segment populations of patients (e.g., with lupus) based on a complement profile that will, for example, provide practitioners with information about which patients are more likely to respond to a certain drug or not, as a screening tool for clinical trial enrollment, and/or predict what type of treatment may benefit a given patient. For example, a patient in the first group where sC5b-9 positively corresponds to renal activity may be more likely to respond to an anti-C5 therapeutic. Thus, the present Example provides a new and unexpected approach to segmenting populations of patients with an autoimmune disease, disorder or condition.

1. A method of measuring a target comprising:
(i) obtaining a sample;
(ii) contacting an immunoassay device with at least a portion of the sample; and
(iii) measuring one or more targets in the sample.

2. The method of claim 1, wherein the immunoassay device comprises at least one test strip.

3. The method of claim 1, wherein the immunoassay device is contacted with a portion of the sample within 30 minutes of a sample being obtained.

4. The method of claim 3, wherein the sample is not subjected to an offline dilution prior to contacting the immunoassay device.

5. The method of claim 1, wherein prior to contacting the immunoassay device, the sample is subjected to a pre-processing step comprising at least one dilution.

6. The method of any one of claims 1-5, wherein the sample is or comprises fluid.

7. The method of any one of claims 1-6, wherein the sample is not solid or substantially comprised of solid material.

8. The method of any one of claims 1-7, wherein the sample is or comprises whole blood, plasma, serum, aqueous humor, tears, ocular fluid, urine and/or cerebrospinal fluid.

9. The method of any one of claims 1-8, wherein the sample is a crude sample.

10. The method of claim 9, wherein the crude sample has not been diluted prior to contacting the immunoassay device.

11. The method of any one of claims 1-10, wherein the sample has undergone one or more purification steps prior to contacting the immunoassay device.

12. The method of any one of claims 1-11, comprising an immunoassay device which comprises at least one of a sample pad and conjugate pad.

13. The method of any one of claims 1-11, wherein the immunoassay device comprises at least two, three, four, five, or six test strips.

14. The method of claim 13, wherein the at least two, three, or four test strips each comprises at least two, three, four, or five test lines.

15. The method of any one of claims 1-14, wherein the measuring is performed between approximately 30-300 minutes after the sample is obtained.

16. The method of any one of claims 1-15, wherein the measuring is performed no greater than 30 minutes after the sample is obtained.

17. In a method of measuring at least one target in a sample, the improvement comprising measuring the target in a sample by contacting an immunoassay device with a sample that has not been subjected to an offline dilution prior to the contacting, and measuring one or more targets, wherein the measurements are more efficient, accurate, sensitive, specific, reliable, and/or rapid as compared to measuring performed with a diluted sample.

18. The method of claim 17, wherein at least one measurement is more efficient, sensitive, accurate, specific, reliable, and/or rapid than a method that comprises one or more offline sample dilution steps prior to the contacting the immunoassay device.

19. The method of claim 17 or 18, wherein the sample is or comprises fluid.

20. The method of any one of claims 17-19, wherein the sample is not solid or substantially comprised of solid material.

21. The method of any one of claims 17-20, wherein the sample is or comprises whole blood, plasma, serum, aqueous humor, urine, tears, ocular fluid, and/or cerebrospinal fluid.

22. The method of any one of claims 17-21, wherein the sample is a crude sample.

23. The method of any one of claims 17-22, wherein the sample has undergone one or more purification steps prior to the contacting the immunoassay device.

24. The method of any one of claims 17-23, wherein the measuring is performed using an immunoassay device comprising at least two, three, or four, five, or six test strips, wherein each test strip comprises at least two test, three, four, or five test lines, and, optionally, wherein at least one test line is a control line.

25. The method of any one of claims 17-24, wherein the measuring is performed between approximately 30-300 minutes after the sample is obtained.

26. The method of any one of claims 17-25, wherein the measuring is performed no greater than 30 minutes after the sample is obtained.

27. A method comprising:
(a) measuring a target in a sample comprising steps of:
(i) obtaining the sample;
(ii) contacting an immunoassay device with at least a portion of the sample;
(iii) allowing the sample and the immunoassay device to sit together for a period of time;
(iv) measuring at least one target in the sample; and
(b) comparing the measurement of one or more targets to at least one reference measurement; and
(c) optionally changing or administering one or more treatments to a subject from whom the sample was obtained.

28. The method of claim 27, wherein the immunoassay device comprises at least one conjugate pad.

29. The method of claim 28, wherein the at least one conjugate pad comprises beads.

30. The method of claim 29, wherein the beads comprise nanobeads.

31. The method of claim 30, wherein the nanobeads are between 100-500 nm.

32. The method of claim 30 or 31, wherein the nanobeads are between 200-400 nm.

33. The method of any one of claims 28-31, wherein the nanobeads comprise at least one detecting agent.

34. The method of claim 33, wherein the at least one detecting agent is a labeled antibody.

35. The method of any one of claims 27-34, wherein the immunoassay device comprises at least one capture agent.

36. The method of claim 35, wherein the at least one capture agent is or comprises an antibody.

37. The method of any one of claims 27-34, wherein the immunoassay device further comprises at least one of a conjugate pad and sample pad.

38. The method of claim 37, wherein the conjugate pad comprises at least one competing agent.

39. The method of any one of claims 35-38, wherein the at least one competing agent binds to at least one target in the sample.

40. The method of any one of claims 35-38, wherein the at least one competing agent binds to an excess amount of at least one target in the sample.

41. The method of any one of claims 27-40, wherein the measuring is performed using a reader system capable of measuring multiple test lines in multiple visualizable channels.

42. A method comprising:
(a) measuring an amount of a target in a sample with steps comprising:
(i) obtaining a sample, wherein the sample is diluted in a vessel;
(ii) allowing the vessel with the diluted sample to sit for a first period of time;
(iii) contacting an immunoassay device with the diluted sample;
(iv) allowing the immunoassay device comprising the diluted sample to sit for a second period of time;
(v) measuring at least one target in the sample; and
(b) optionally changing or administering one or more treatments to a subject from whom the sample was obtained.

43. The method of claim 42, wherein the sample is diluted with a substrate with which at least one target in the sample can react.

44. The method of claim 42, wherein the first period of time is no longer than five minutes.

45. The method of claim 42, wherein the second period of time is no longer than five minutes.

46. A kit comprising:
(i) a test cassette comprising an immunoassay device, which immunoassay device comprises at least one test strip comprising at least one test line;
(ii) at least one detecting agent; and, optionally
(iii) at least one competing agent.

47. A test cassette comprising:
(i) an immunoassay device, which itself comprises at least one test strip; and
(ii) at least two test lines.

48. A method of diagnosing a subject as having or being susceptible to having at least one disease, disorder, or condition comprising:
(i) obtaining a sample from the subject;
(ii) measuring one or more targets in the sample;
(iii) comparing the measurement of one or more targets to a measurement range of one or more reference samples; and
(iv) diagnosing a patient as having or being at risk of developing a disease, disorder or condition if a measurement of the one or more targets is outside of the measurement range of the one or more reference samples.

49. A reader system comprising:
(a) means for measuring one or more targets in a sample comprising
(i) inserting a test cassette into the reader system such that the direction of sample flow on an immunoassay device of the test cassette is oriented parallel with gravity
(ii) measuring at least one target on at least one test line on at least one test strip in the immunoassay device.

50. The reader system of claim 49, wherein the test cassette is inserted via a port in the reader system.

51. The reader system of claim 49 or 50, wherein the sample is added to the immunoassay device prior to insertion of the test cassette into the reader system.

52. The reader system of claim 49 or 50, wherein the sample is added to the immunoassay device after insertion of the test cassette into the reader system.

53. The reader system of any of claims 49-52, wherein the measuring comprises measuring at least four test lines on at least one test strip of the immunoassay device.

54. The method of any of claim 1-45 or 48, wherein the target is one or more of C3, C3a, iC3b, C4, C5, sC5b-9, IL-6, ADAMTS13, MASP2:AT complex, C4d, Ba, Bb, FH, CXCL9, sCD25, microRNA, IL8, Pentraxin3, IL1, VCAM1, thrombomodulin, ferritin, CRP, IL-10, TNFα, IFNγ, and/or creatinine.

55. The kit of claim 46, wherein the at least one test line comprises at least one capture agent for one or more of C3, C3a, iC3b, C4, C5, sC5b-9, IL-6, ADAMTS13, MASP2:AT complex, C4d, Ba, Bb, FH, CXCL9, sCD25, microRNA, IL8, Pentraxin3, IL1, VCAM1, thrombomodulin, ferritin, CRP, IL-10, TNFα, IFNγ, and/or creatinine.

56. The test cassette of claim 47, wherein at least one test line comprises at least one capture agent for one or more of C3, C3a, iC3b, C4, C5, sC5b-9, IL-6, ADAMTS13, MASP2:AT complex, C4d, Ba, Bb, FH, CXCL9, sCD25, microRNA, IL8, Pentraxin3, IL1, VCAM1, thrombomodulin, ferritin, CRP, IL-10, TNFα, IFNγ, and/or creatinine.

57. The reader system of any one of claims 49-53, wherein the one or more targets comprises one or more of C3, C3a, iC3b, C4, C5, sC5b-9, IL-6, ADAMTS13, MASP2:AT complex, C4d, Ba, Bb, FH, CXCL9, sCD25, microRNA, IL8, Pentraxin3, IL1, VCAM1, thrombomodulin, ferritin, CRP, IL-10, TNFα, IFNγ, and/or creatinine.

58. The method of any one of claim 27, 42, or 48, wherein the subject is suspected or at risk of having one or more of age-related macular degeneration (AMD), complement 3 glomerulopathy (C3G), Hematopoietic Stem Cell Transplant-Associated Thrombotic Microangiopathy (HSCT-TMA), Complement-Mediated Thrombotic Microangiopathy (CM-TMA), atypical hemolytic uremic syndrome (aHUS), thrombotic thrombocytopeniarpura (TTP), COVID19, lupus erythematosus, lupus nephritis, cytokine release syndrome, Alzheimer's Disease (AD), or combinations thereof.

59. The method of any one of claim 27, 42, or 48, wherein the subject is diagnosed as at risk of developing or as having one or more of-related macular degeneration (AMD), complement 3 glomerulopathy (C3G), Hematopoietic Stem Cell Transplant-Associated Thrombotic Microangiopathy (HSCT-TMA), Complement-Mediated Thrombotic Microangiopathy (CM-TMA), atypical hemolytic uremic syndrome (aHUS), thrombotic thrombocytopenia purpura (TTP), COVID19, lupus erythematosus, lupus nephritis, cytokine release syndrome, Alzheimer's Disease (AD), or combinations thereof.
